(19) United States
(12) Patent Application Publication
(10) Pub. No.: US 2022/0175675 A1
(43) Pub. Date: Jun. 9, 2022

(54) ENCAPSULATING MOLECULES IN FAT GLOBULES

(71) Applicant: The Regents of the University of California, Oakland, CA (US)

(72) Inventors: Nitin NITIN, Oakland, CA (US); Maha ALSHEHAB, Oakland, CA (US)

(21) Appl. No.: 17/594,825

(22) PCT Filed: Apr. 30, 2020
(86) PCT No.: PCT/US2020/030850

§ 371 Date: Oct. 29, 2021

Related U.S. Application Data
(60) Provisional application No. 62/843,243, filed on May 3, 2019.

(51) Int. Cl. A61K 9/127; A61K 31/593; A61K 31/353; A61K 31/085; A61K 31/232; A61K 31/12; A23L 27/00; A23L 33/105; A23L 33/155; A23L 27/10; A23P 10/35

(52) U.S. Cl. CPC A61K 9/1276; A61K 31/593; A61K 31/353; A61K 31/085; A61K 31/232; A23V 2002/00; A23L 27/72; A23L 33/105; A23L 33/155; A23L 27/10; A23P 10/35; A61K 31/12

(57) ABSTRACT

Provided herein are methods of encapsulating molecules in milk fat globules and oleosomes, and compositions and pharmaceutical formulations of compositions with molecules encapsulated in milk fat globules and oleosomes.

### PRIORITY AND CROSS REFERENCE TO RELATED APPLICATIONS

This application claims the benefit of U.S. Provisional Application 62/843243 filed on May 3, 2019, which is hereby incorporated by reference in its entirety.

### STATEMENT REGARDING FEDERALLY SPONSORED RESEARCH OR DEVELOPMENT

This invention was made with Government support under Grant No. 2016-67017-24603 awarded by the USDA Foundational Food Quality Program. The Government has certain rights in this invention.

### BACKGROUND

### Field

The present disclosure is generally related to methods of encapsulating molecules in milk fat globules and oleosomes, and compositions comprising molecules encapsulated in milk fat globules and oleosomes.

### Description of the Related Art

Encapsulation using naturally occurring and pre-formed carriers has been of interest for food, agricultural, cosmetic, and medical applications in recent years. In order for compounds of interest to be encapsulated in pre-formed structures, they must partition via the interface. Naturally occurring interfaces are often highly complex compositionally and structurally, thus their permeability behavior deviated from engineered interfaces used to model and predict partitioning of common compounds of interest such as lipophilic bioactives.

Stability and dispersibility of lipid-soluble bioactives in food systems are often modified via encapsulation in oil in water emulsions and lipid or protein-based nanoparticles. These encapsulation approaches frequently require the addition of surfactants/emulsifiers, sacrificial antioxidants, and high-energy methods to create the dispersed phase and stabilize encapsulated compounds.

Vitamin D₃ (cholecalciferol) is a fat-soluble compound that is required for optimal human health, and must be obtained from exogenous sources. Encapsulation of VD₃ is a leading approach for its delivery in many food and drug formulations.

Optimal curcumin delivery in food and pharmaceutical applications requires an encapsulation system that can render it good stability against chemical and physical stress in the processing conditions as well as in gastrointestinal tract.

### SUMMARY

In some embodiments, a method for encapsulating at least one compound, comprises providing the at least one compound, mixing a plurality of milk fat globules or oleosomes with the at least one compound to obtain a mix, incubating the mix to allow the at least one compound to diffuse into at least one substructure of a fraction of the plurality of the milk fat globules or oleosomes comprising the at least one compound.

In some embodiments of a method for encapsulating at least one compound, the at least one compound is lipophilic or amphiphilic.

In some embodiments of a method for encapsulating at least one compound, the at least one compound is one or more of a bio-active compound, a colorant, or a flavor.

In some embodiments of a method for encapsulating at least one compound, the at least one compound is provided in a pharmaceutically acceptable solvent or food-grade solvent.

In some embodiments of a method for encapsulating at least one compound, the pharmaceutically acceptable solvent or food-grade solvent is selected from the group consisting of an organic solvent, a polar solvent, and a volatile oil.

In some embodiments of a method for encapsulating at least one compound, the at least one compound is provided as a micellar composition that fuses with the milk fat globule or oleosome.

In some embodiments of a method for encapsulating at least one compound, the plurality of milk fat globules or oleosomes is provided in water.

In some embodiments of a method for encapsulating at least one compound, the fraction of the plurality of milk fat globules or oleosomes comprising the at least one compound ranges from about 30% to about 100%.

In some embodiments of a method for encapsulating at least one compound, the milk fat globules are isolated from milk selected from the group consisting of bovine milk, ovine milk, goat milk, camel milk, and yak milk.

In some embodiments of a method for encapsulating at least one compound, the oleosome is from one or more of seeds, or nuts.

In some embodiments of a method for encapsulating at least one compound, the milk fat globules or oleosomes are isolated by one or more of density, size, or polarity based methods.

In some embodiments of a method for encapsulating at least one compound, the density based method comprises centrifugation.

In some embodiments of a method for encapsulating at least one compound, the size based comprises filtration.

In some embodiments of a method for encapsulating at least one compound, the polarity based method comprises membrane and electric field induced separation methods.

In some embodiments of a method for encapsulating at least one compound, a concentration of pharmaceutically acceptable solvent or food-grade solvent ranges from about 0.1% v/v to about 10% v/v.

In some embodiments of a method for encapsulating at least one compound, the pharmaceutically acceptable solvent or food-grade solvent is ethanol.

In some embodiments of a method for encapsulating at least one compound, a concentration of ethanol is <10% v/v.

In some embodiments of a method for encapsulating at least one compound, the at least one compound has a log P value ranging from about 0.5 to about 10.

In some embodiments of a method for encapsulating at least one compound, the at least one compound has a molecular mass ranging from about 50 g/mol to about 5000 g/mol.

In some embodiments of a method for encapsulating at least one compound, the at least one compound is a food compound selected from the group consisting of lipid soluble vitamins (Vitamin A, D and K and their derivatives), flavors (Eugenol, Limonene, Vanillin, Rosemary, Dairy Flavors, and the like), Colors (Curcumin, annatto extract, capsaicin, and the like), Polyphenolic antioxidants (Flavonoids, Anthocynanins, Proanthocyanidins, Curcuminoids, carotenoids, retinoids).

In some embodiments of a method for encapsulating at least one compound, the at least one compound is a pharmaceutical compound selected from the group consisting of HIV drugs, Cardiovascular drugs, antibiotic and antifungals, and oral anti-cancer drugs.

In some embodiments of a method for encapsulating at least one compound, the encapsulation of the at least one compound is quantified by a method selected from the group consisting of fluorescence microscopy, High Performance Liquid Chromatography, Liquid chromatography—mass spectrometry, High Performance Liquid chromatography—mass spectrometry, Gas chromatography—mass spectrometry, ultraviolet—visible spectroscopy or ultraviolet—visible spectrophotometry, ultraviolet—visible-near infrared spectroscopy or ultraviolet—visible spectrophotometry, Raman spectroscopy, and Fourier-transform infrared spectroscopy.

In some embodiments of a method for encapsulating at least one compound, the at least one compound displays a ring like distribution, peripheral distribution, homogenous distribution, or a combination thereof within the substructure of fraction of the plurality of milk fat globules.

In some embodiments of a method for encapsulating at least one compound, the incubating is performed a temperature range of about 4° C. to about 40° C.

In some embodiments of a method for encapsulating at least one compound, the incubating is performed a temperature range of about −10° C. to about 90° C.

In some embodiments of a method for encapsulating at least one compound, the incubating is performed for about 30 sec to about 50 h.

In some embodiments of a method for encapsulating at least one compound, the incubating is performed for about 10 min to about 60 min.

In some embodiments of a method for encapsulating at least one compound, the at least one compound is encapsulated at a concentration range of about 5 μg/g of milk fat to about 100 μg/g of milk fat.

In some embodiments of a method for encapsulating at least one compound, the at least one compound is encapsulated at a range of about 10% to about 100% of the dosage requirement per gm of milk fat.

In some embodiments of a method for encapsulating at least one compound, the at least one compound is provided at a concentration range of about 50 μM to about 2500 μM.

In some embodiments of a method for encapsulating at least one compound, the at least one compound is provided at a dose of about 0.01 mg/kg/day to about 1000 mg/kg/day.

In some embodiments, a composition comprises a plurality of milk fat globules or oleosomes, at least one compound, an amount of a pharmaceutically acceptable solvent or food-grade solvent, wherein the at least one compound is encapsulated within a fraction of the plurality of milk fat globules or oleosomes.

In some embodiments of a composition, the at least one compound is lipophilic or amphiphilic.

In some embodiments of a composition, the at least one compound is one or more of a bio-active compound, a colorant, or a flavor.

In some embodiments of a composition, the at least one compound is provided in a pharmaceutically acceptable solvent or food-grade solvent.

In some embodiments of a composition, the pharmaceutically acceptable solvent or food-grade solvent is selected from the group consisting of an organic solvent, a polar solvent, and a volatile oil.

In some embodiments of a composition, a concentration of pharmaceutically acceptable solvent or food-grade solvent ranges from about 0.1 -10% v/v.

In some embodiments of a composition, the pharmaceutically acceptable solvent or food-grade solvent is ethanol.

In some embodiments of a composition, a concentration of ethanol is ≤10% v/v.

In some embodiments of a composition, the at least one compound has a log P value ranging from about 0.5 to about 10.

In some embodiments of a composition, the at least one compound has a molecular mass ranging from about 50 g/mol to about 5000 g/mol.

In some embodiments of a composition, the compound is a food compound selected from the group consisting of lipid soluble vitamins (Vitamin A, D and K and their derivatives), flavors (Eugenol, Limonene, Vanilin, Rosemary, Dairy Flavors . . . and several others), Colors (Curcumin, annatto extract, capsaicin and several others), Polyphenolic antioxidants (Flavonoids, Anthocynanins, Proanthocyanidins, Curcuminoids, carotenoids, retinoids).

In some embodiments of a composition, the compound is a pharmaceutical compound selected from the group consisting of HIV drugs, Cardiovascular drugs, antibiotic and antifungals, and oral anti-cancer drugs.

In some embodiments of a composition, the at least one compound is encapsulated at a concentration range of about 5 μg/g of milk fat to about 100 μg/g of milk fat.

In some embodiments of a composition, the at least one compound is provided at a concentration range of about 50 μM to about 2500 μM.

In some embodiments of a composition, the at least one compound is encapsulated at a range of about 10% to about 100% of the dosage requirement per gm of milk fat.

In some embodiments of a composition, a size of the fraction of the plurality of milk fat globules or oleosomes comprising the at least one compound ranges from about 5 um to about 50 um.

In some embodiments of a composition, a size of the fraction of the plurality of milk fat globules or oleosomes comprising the at least one compound ranges from about 0.1 um to about 50 um.

In some embodiments of a composition, a size of the fraction of the plurality of milk fat globules or oleosomes comprising the at least one compound ranges from about 0.1 um to about 10 um.

In some embodiments of a composition, the composition is for one or more of oral administration, transdermal administration, and topical administration.

In some embodiments of a composition, the milk fat globules or oleosomes are present at a concentration of about 0.1% w/v to about 50% w/v.

In some embodiments, a composition comprises a plurality of milk fat globules or oleosomes, at least one compound, an amount of a pharmaceutically acceptable solvent or food-grade solvent, wherein the at least one compound is encapsulated within a fraction of the plurality of milk fat globules or oleosomes, and wherein the composition is configured to deliver the compound at a site of interest.

In some embodiments of a composition, the composition is configured to deliver the compound at a site of interest by gradually releasing the compound over a period of time.

In some embodiments of a composition, the period of time ranges from about 2 h to about 24 h.

In some embodiments of a composition, the period of time ranges from about 1 min to about 6 h.

In some embodiments of a composition, the composition is configured to deliver the compound at a site of interest by immediately releasing the compound.

In some embodiments of a composition, the fraction of the plurality of milk fat globules or oleosomes comprising the at least one compound ranges from about 30% to about 100%.

In some embodiments of a composition, the site of interest is stomach, small intestine, large intestine, liver, skin, oral cavity, and teeth.

In some embodiments of a composition, the at least one compound encapsulated within the plurality of milk fat globules or oleosomes is stabilized against acid degradation.

In some embodiments of a composition, the at least one compound encapsulated within the plurality of milk fat globules or oleosomes is stabilized against acid degradation at a pH of about 0.5 to about 5.

In some embodiments of a composition, a degradation/loss of activity one compound encapsulated within the plurality of milk fat globules or oleosomes under acidic pH conditions is determined by measuring a degradation product of the at least one compound.

In some embodiments of a composition, a degradation/loss of activity one compound encapsulated within the plurality of milk fat globules or oleosomes under acidic pH conditions is determined by measuring a degradation product of the at least one compound by LC-MS.

In some embodiments of a composition, a size of the fraction of the plurality of milk fat globules or oleosomes comprising the at least one compound ranges from about 5 um to about 50 um.

In some embodiments of a composition, a size of the fraction of the plurality of milk fat globules or oleosomes comprising the at least one compound ranges from about 0.1 um to about 50 um.

In some embodiments of a composition, a size of the fraction of the plurality of milk fat globules or oleosomes comprising the at least one compound ranges from about 0.1 um to about 10 um.

In some embodiments of a composition, the composition is for one or more of oral administration, transdermal administration, and topical administration.

In some embodiments of a composition, about 10% to about 80% of the at least one compound is released from the fraction of the plurality of milk fat globules comprising the at least one compound.

In some embodiments of a composition, the at least one compound is provided at a dose of about 0.01 mg/kg/day to about 1000 mg//kg/day.

In some embodiments, a composition comprises at least one compound encapsulated within a fraction of the plurality of milk fat globules or oleosomes by a method, comprising providing a plurality of milk fat globules or oleosomes, providing at least one compound, mixing the plurality of milk fat globules or oleosomes with the at least one compound to obtain a mix, incubating the mix to allow the at least one compound to diffuse into at least one substructure of a fraction of the plurality of milk fat globules or oleosomes comprising the at least one compound, thereby encapsulating the at least one compound, wherein the composition is configured to deliver the compound at a site of interest.

In some embodiments of a composition, the composition provides thermal stability, oxidative stability, improved delivery, light stability, and pH stability to the at least one compound.

In some embodiments of a composition, the composition provides biocompatibility to the at least one compound.

In some embodiments of a composition, the composition provides extended stability to the at least one compound.

In some embodiments of a composition, the composition reduces a toxicity of the at least one compound.

In some embodiments of a composition, the composition provides a controlled release of the at least one compound.

In some embodiments, a composition comprises an intact milk fat globule or oleosome, at least one compound, wherein the at least one compound is encapsulated within the intact milk fat globule.

In some embodiments of a composition, the compound comprises at least one of a protein, an antioxidant, and an enzyme.

In some embodiments of a composition, the composition is stable during gastric pass.

In some embodiments, a pharmaceutical formulation comprises one or more of the compositions provided herein, and a pharmaceutically acceptable carrier.

### BRIEF DESCRIPTION OF THE DRAWINGS

FIG. 1A shows cross sections of MFGs with indigenous fluorescence of encapsulated lipophilic compounds Quercetin and Fisetin.

FIG. 1B shows cross sections of MFGs with indigenous fluorescence of encapsulated lipophilic compounds retinyl acetate, cholecalciferol, and eugenol.

FIG. 2A shows droplet size distribution of MFGs of bovine milk. The results show the droplet diameter in raw milk and after isolation via sequential centrifugation and washing steps.

FIG. 2B shows droplet size distribution of MFGs of ovine milk. The results show the droplet diameter in raw milk and after isolation via sequential centrifugation and washing steps.

FIGS. 3A and 3B show fluorescence imaging of MFGs to demonstrate the strucutre' s intergirty following treatment with 1 and 10% v/v ethanol.

FIG. 3A shows bright field and fluorescence images of Oregon Green™ 488 DHPE probed MFGM under various ethanol treatment. Images are captured at 1.25 digital zoom, with 3 line averages.

FIG. 3B shows Z-stack of FIG. 3A showing stainig of the MFGM. Control and 1% v/v ethanol are captured at 2.5 digital zoom, and 10% v/v ethanol were captured at 3.5 digital zoom. Variations in particle distribution through the imaging field are caused by inconsistency in aliquot preparation as separated cream is diluted in water and glycerol for imaging purposes, and does not represent an effect of ethanol treatment.

FIG. 4 shows the influence of incubation time on encapsualtion yield of VD3 into bovine MFGs. * indicate statistically significant increase in LY (μg of VD₃/g of bovine milk fat)

FIGS. 5A-5C show localization of VD₃ in MFGs post encapsulation.

FIG. 5A shows multiphoton fluorescence images of VD₃ encapsulated in in MFGs. Image is captured at 1.25 digital zoom, with 8 line averages.

FIG. 5B shows Z-stack (24 frames captured at 3.5 digital zoom with a total thickness 9.79 μm) illustrate 3-D distribution of VD3 encapsulated in MFGs.

FIG. 5C shows average line scans of 6 representative particles showing uniform fluorescent signal distribution indicating non-specific localization of VD₃ in the lipid core of MFGs.

FIG. 6 shows LC-MS results of degradation of VD₃ under gastric-relevant pH values.

FIG. 7 shows LC-MS profile of acid degradation products of VD₃ after 2 h incubation under in vitro gastric conditions. Right coloumn represents VD3 encapsulated in MFGs, and the left column represents free VD₃.

FIGS. 8A and 8B show confocal laser scanning microscopy showing the partitioning of curcumin into MFGs after 10 minutes incubation.

FIG. 8A shows bright field image of MFGs with encapsulated curcumin.

FIG. 8B shows fluorescence images of curcumin in MFGs.

FIG. 9 shows release of curcumin encapsulated in MFGs under simulated gastric conditions.

FIGS. 10A-10D show confocal laser scanning microscopy images of MFGs with encapsulated curcumin during simulated gastric digestion.

FIGS. 10A and 10B show bright field and fluorescence images after 30 min of SGD, respectively. MFGs appear spherical and intact with uniformly distributed fluorescence signal of curcumin. SGD, simulated gastric digestion crystalline lipids.

FIGS. 10C and 10D show show bright field image of MFGs after 120 min of SGD. Red arrows point at Irregular boundaries of the lipid droplets. SGD, simulated gastric digestion crystalline lipids.

FIG. 11 shows MFGs diameter distribution prior to digestion (circle), post gastric digestion (square), and post intestinal digestion (triangle).

FIG. 12 shows release of curcumin encapsulated in MFGs under simulated intestinal conditions.

FIGS. 13A-13D show confocal laser scanning microscopy images of MFGs with encapsulated curcumin during simulated intestinal digestion.

FIG. 13A shows bright field images captured after 30 min of SID, blue arrow point at oil droplets being expelled, red arrows in point at amorphous lipids.

FIG. 13B shows fluorescence images at 30 min, fluorescence signal indicate presence of curcumin SID, simulated intestinal digestion.

FIG. 13C shows red arrows point in at needle-shaped structures.

FIG. 13D shows fluorescence images at 180 min, fluorescence signal indicate presence of curcumin SID, simulated intestinal digestion.

FIGS. 14A-14D show confocal laser scanning microscopy images of MFGs before and after treatment with 10% v/v ethanol Images captures at 3.5 digital zoon.

FIGS. 14A and 14B show MFGs with Oregon Green™ 488 DHPE probed MFGM in the absence of ethanol.

FIGS. 14C and 14D show MFGs after 30 min incubation in the presence of 10% v/v ethanol in the aqueous phase. Note: variations in in MFGs density within each imaging field is caused by aliqouate preparation inconsistency as cream is diluted in water and glycerol for imaging purposes.

FIG. 15A shows curcumin encapsulation efficiency in oleosome with different incubation times.

FIG. 15B shows confocal images of oleosome after encapsulation.

FIG. 16 shows curcumin release in simulated gastric digestion.

FIG. 17 shows zeta-potential of oleosome and WPI before and after digestion.

FIG. 18 shows a schematic of an embodiment of an encapsulation process according to the present disclosure.

FIG. 19 shows data related to physical stability of milk fat globules. Results show that that milk fat globules retain most of their stability even after several washes.

FIG. 20 shows data related to loading of VD₃ in milk fat globules.

FIG. 21 shows data related to localization of DMEQ-TAD-labelled-VD3 in milk fat globules.

FIG. 22 shows a distribution of 3000 common drugs according to their biopharmaceutical classification based on the biopharmaceutical classification system (BCS) and based on their Log P (Log P_{ow}) value

FIG. 23 shows data related to localization of various labelled bioactive compounds.

### DETAILED DESCRIPTION

In some embodiments, the present disclosure is related to methods of encapsulating, stabilizing, and delivering bio-active compounds, vitamins, fatty acids, and other small lipid-soluble molecules using at least milk fat globules and oleosomes and compositions obtained by said methods. In some embodiments, the present disclosure is provides milk fat globules and oleosomes as a universal delivery system. In some embodiments, the present disclosure provides methods of encapsulation, improved loading and dosage of encapsulated compounds, improved stability of encapsulated compounds, and improved bioaccessibility via gastrointestinal route.

### Methods for Encapsulating Compounds

In some embodiments, a method for encapsulating at least one compound comprises providing the at least one compound, mixing a plurality of milk fat globules or oleosomes with the at least one compound to obtain a mix, incubating the mix to allow the at least one compound to diffuse into at least one substructure of a fraction of the plurality of the milk fat globules or oleosomes comprising the at least one compound.

In some embodiments of a method for encapsulating at least one compound, the at least one compound is lipophilic or amphiphilic.

In some embodiments of a method for encapsulating at least one compound, the at least one compound is one or more of a bio-active compound, a colorant, or a flavor.

In some embodiments of a method for encapsulating at least one compound, the at least one compound is provided in a pharmaceutically acceptable solvent or food-grade solvent. Non-limiting examples of pharmaceutically acceptable solvents or food-grade solvents include acetone, Benzyl Alcohol, 1,3-Butylene Glycol, Carbon Dioxide, Castor Oil, Citric Acid, Esters of Mono- and Di-glycerides, Ethyl Acetate, Ethyl Alcohol (Ethanol), Ethyl alcohol denatured with methanol, Glycerol (Glycerin), Glyceryl diacetate, Glyceryl triacetate (Triacetin), Glyceryl tributyrate (Tributyrin), Hexane, Isopropyl alcohol (Isopropanol), Methyl Alcohol (methanol), Methyl ethyl ketone (2-Butanone), Methylene Chloride (Dichloro-methane), Monoglycerides and diglycerides, Monoglyceride citrate, 2-Nitropropane, 1,2-Propylene glycol (1,2-propanediol), Propylene glycol mono-esters and diesters of fat-forming fatty acids, and Triethyl citrate.

In some embodiments of a method for encapsulating at least one compound, the pharmaceutically acceptable solvent or food-grade solvent is selected from the group consisting of an organic solvent, a polar solvent, and a volatile oil.

In some embodiments, the pharmaceutically acceptable solvent or food-grade solvent is not water as found in milk. In some embodiments, the pharmaceutically acceptable solvent or food-grade solvent is water that is not contained in or as a part of milk. In some embodiments, the solvent is one or more of distilled water, deionized water, desalinated water, purified water, or ultrapure water. In some embodiments, the pharmaceutically acceptable solvent or food-grade solvent is from an external source. In some embodiments, the pharmaceutically acceptable solvent or food-grade solvent is not from or has been processed by a mammal In some embodiments, the pharmaceutically acceptable solvent or food-grade solvent is from a source separate from the source of the milk. In some embodiments, the pharmaceutically acceptable solvent or food-grade solvent is not the same water that constitutes the water in milk as originally produced by the mammal.

In some embodiments, the at least one compound is not found in milk. In some embodiments, the at least one compound is not contained in or as a part of milk. In some embodiments, the at least one compound is from an external source. In some embodiments, the at least one compound is from a source that is separate from the source of the milk.

In some embodiments of a method for encapsulating at least one compound, the at least one compound is provided as a micellar composition that fuses with the milk fat globule or oleosome.

In some embodiments of a method for encapsulating at least one compound, the plurality of milk fat globules or oleosomes is provided in water.

In some embodiments, the percent of water ranges from about 99% to about 90%. In some embodiments, the percent of water ranges from about 95% to about 80%. In some embodiments, the percent of water ranges from about 85% to about 70%. In some embodiments, the percent of water ranges from about 75% to about 60%. In some embodiments, the percent of water ranges from about 65% to about 50%. In some embodiments, the water is one or more of distilled water, deionized water, desalinated water, purified water, or ultrapure water. In some embodiments, the water is from an external source. In some embodiments, the water is not from or has been processed by a mammal. In some embodiments, the water is from a source separate from the source of the milk. In some embodiments, the water is not the same water that constitutes the water in milk as originally produced by the mammal.

In some embodiments of a method for encapsulating at least one compound, the fraction of the plurality of milk fat globules comprising the at least one compound ranges from about 30% to about 100%. In some embodiments, the fraction of the plurality of milk fat globules comprising the at least one compound ranges from about 60% to about 100%. In some embodiments, the fraction of the plurality of milk fat globules comprising the at least one compound ranges from about 90% to about 100%. In some embodiments, the fraction of the plurality of milk fat globules comprising the at least one compound is 100%.

In some embodiments of a method for encapsulating at least one compound, the milk fat globules are isolated from one or more of bovine milk, ovine milk, goat milk, camel milk, or yak milk.

In some embodiments of a method for encapsulating at least one compound, the oleosome is from one or more of seeds, or nuts.

In some embodiments of a method for encapsulating at least one compound, the milk fat globules or oleosomes are isolated by one or more of density, size, or polarity based methods. In some embodiments of a method for encapsulating at least one compound, the density based method comprises centrifugation. In some embodiments of a method for encapsulating at least one compound, the size based comprises filtration. In some embodiments of a method for encapsulating at least one compound, the polarity based method comprises membrane and electric field induced separation methods.

In some embodiments of a method for encapsulating at least one compound, a concentration of pharmaceutically acceptable solvent or food-grade solvent ranges from about 0.1% v/v to about 10% v/v. In some embodiments, a concentration of pharmaceutically acceptable solvent or food-grade solvent ranges from about 0.5% v/v to about 15% v/v. In some embodiments, a concentration of pharmaceutically acceptable solvent or food-grade solvent ranges from about 1% v/v to about 20% v/v. In some embodiments, a concentration of pharmaceutically acceptable solvent or food-grade solvent ranges from about 0.01% v/v to about 25% v/v.

In some embodiments of a method for encapsulating at least one compound, the pharmaceutically acceptable solvent or food-grade solvent is ethanol. In some embodiments, a concentration of ethanol is <10% v/v. In some embodiments, a concentration of ethanol ranges from about 0.01% v/v to about 10% v/v.

In some embodiments of a method for encapsulating at least one compound, the at least one compound has a Log P value (or low P_{ow} value) ranging from about 0.5 to about 10. In some embodiments, the at least one compound has a Log P value ranging from about 0.1 to about 10. Log P value is used as a measure of lipophilicity, which is the partition coefficient of a molecule between an aqueous and lipophilic phases, usually octanol and water. Log P value can be measured in a variety of ways known in the art. For example, the most common method is the shake-flask method, which consists of dissolving some of the solute in question in a volume of octanol and water, shaking for a period of time, then measuring the concentration of the solute in each solvent.

In some embodiments of a method for encapsulating at least one compound, the at least one compound has a molecular mass ranging from about 50 g/mol to about 5000 g/mol. In some embodiments, the at least one compound has a molecular mass ranging from about 5 g/mol to about 50,000 g/mol.

In some embodiments of a method for encapsulating at least one compound, the at least one compound is a food compound is one or more lipid soluble vitamins Non-limiting examples include Vitamin A, Vitamin D, Vitamin K, and their derivatives. In some embodiments of a method for encapsulating at least one compound, the at least one compound is one or more flavors. Non-limiting examples include Eugenol, Limonene, Vanillin, Rosemary, Dairy Flavors, and the like. In some embodiments of a method for encapsulating at least one compound, the at least one compound is one or more colors. Non-limiting examples include curcumin, annatto extract, capsaicin, and the like. In some embodiments of a method for encapsulating at least one compound, the at least one compound is one or more polyphenolic antioxidants. Non-limiting examples include Flavonoids, Anthocynanins, Proanthocyanidins, Curcuminoids, carotenoids, and retinoids. In some embodiments, the at least one compound is fisetin, quercetin hydrate, eugenol, colecalciferol, retinyl acetate, and curcumin.

In some embodiments of a method for encapsulating at least one compound, the at least one compound is a pharmaceutical compound selected from the group consisting of HIV drugs, Cardiovascular drugs, antibiotic and antifungals, and oral anti-cancer drugs. Non-limiting examples are shown in Tables 0.1 and 0.2.

Table 0.1 shows a list of commonly used cardiovascular drugs for which a licensed liquid is available in the UK.

TABLE 0.1

| Drug | Pediatric license | Description | Remarks |
|---|---|---|---|
| Amiloride | yes | Sugar-free oral solution | Contains propylene glycol |
| Atenolol | No | Syrup | Commercial preparation licensed for adults. Not |
| | | | recommended for use in children by manufacturer. |
| Digoxin | Yes | Elixer | Contains ethanol and propylene glycol |
| Flecainide | Yes | Syrup | Drug licensed for children over 12 years old |
| Furosemide | Yes | Sugar-free oral solution | Range of strengths available. All strengths contain |
| | | | ethanol and propylene glycol |
| Propranolol | Yes | Sugar-free oral solution | Contains propylene glycol |

Table 0.2 shows a list of commonly used cardiovascular drugs for which no licensed liquid is available in the UK.

TABLE 0.2

| Drug | Pediactric license | Special available | Remarks |
|---|---|---|---|
| Amiodarone | No | Yes (suspension) | Drug sparingly soluble in water. Special only has 1-month shelf-life. |
| | | | Extemporaneous preparation can be made (suspension from tablets) |
| Amlodipine | No | Yes (suspension) | Drug sparingly soluble in water. Special only has 1-month shelf-life. |
| | | | Crushed tablets suspended in water often used |
| Aspirin | No | No | Very water soluble drug-use dispersible tablets |
| Bosentan | No | No | Crushed tablets suspended in water-very expensive |
| Captopril | No | Yes (solution and | Solution-must be refrigerated, only 1-month shelf-life. Licensed |
| | | 2 mg tablets) | solution in Australia, packed under nitrogen with only 1-month |
| | | | shelf-life once opened. Easy dispersible low strength tablets crushed |
| | | | and mixed in water (these have recently been withdrawn from the |
| | | | market) |
| Carvedilol | No | Yes (suspension) | Drug sparingly soluble in water. Special only has 1-month shelf-life. |
| | | | Crushed tablets suspended in water often used |
| Clonidine | No | Yes | Dilution in water of the injection is often used, must be refrigerated. |
| | | | Special has a 1-month shelf-life |
| Enalapril | No | Yes (suspension) | Drug sparingly soluble in water. Crushed tablets suspended in water |
| | | | often used |
| Hydralazine | No | Yes (soluble tablets) | Soluble tablets available. The injection can be diluted and used orally |
| | | | and kept 24 h at room temperature |
| Nadolol | No | Yes (suspension) | Drug sparingly soluble in water. Special only has 1-month shelf-life. |
| | | | Crushed tablets suspended in water often used. Low strength tablets |
| | | | recently withdrawn in UK |
| Nifedipine | No | No | Drops in macrogol 200 can be imported-Crushed modified release |
| | | | tablets or removal of nifedipine liquid from soft capsules used |
| Pravastatin | No | Yes | Drug freely soluble in water, crushed tablets often dissolved in water. |
| | | | Special has 1-month shelf-life |
| Prazosin | Yes (>12 years old) | No | Manipulated solid oral dosage form suspended in water |
| Ramipril | No | Yes (suspension) | Drug sparingly soluble in water. Crushed tablets suspended in water |
| | | | often used. Special only has 1-month shelf-life |
| Sildenafil | No | No | Crushed tablets suspended in water -expensive |
| Spironolactone | No | Yes | Large range of strengths available |
| Warfarin | No | Yes | Drug freely soluble in water, crushed tablets often dissolved in water. |
| | | | Special only has 1-month shelf-life |

Efavirenz (EFV) is a first-line ARV recommended by the WHO for children older than 3 years. Efavirenz is a non-nucleoside reverse transcriptase inhibitor (NNRTI) and is used as part of highly active antiretroviral therapy (HAART) for the treatment of a human immunodeficiency virus (HIV) type 1. Efavirenz is sold as capsule or tablet, one liquid formulation is available: Stocrin, 30 mg/ml manufactured by Merck Sharp & Dohme Limited. Efavirenz is an aromatic heteropolycyclic compound with properties listed in Table 0.3.

TABLE 0.3

| Property | Value |
|---|---|
| Molecular weight | 315.675 g/mol |
| Log P | 3.89-4.46 |
| Hydrogen Acceptor-Donor Count | 2-1 |
| Waters solubility | 0.00855 mg/mL |

In some embodiments, milk fat globules are isolated from raw milk (e.g., bovine milk) via density, size, and/or polarity based separation methods. Lipid-soluble compounds are prepared in a food-grade organic solvent (e.g., ethanol). Milk fat globules suspended in water are incubated with the lipid-soluble compounds at low concentrations of the food-grade solvent (e.g., <10% v/v ethanol). Due to hydrophobic affinity of molecules and the permeability of the milk fat globule membrane, these molecules diffuse to various substructure of the milk fat globules based on their physico-chemical properties. Fluorescent imaging is used to determine the localization of some compounds; some compounds localize at the interface of milk fat globules and milk fat globule membrane, and others demonstrate uniform distribution throughout the milk fat globules. Encapsulated compounds were stabilized against degradation conditions (e.g., gastric acidic conditions), and exhibited gradual release in in vitro gastrointestinal digestion study. In some embodiments, the structural and compositional complexity of milk fat globule will provide controlled release and improved chemical stability of encapsulated compounds using isolated intact milk fat globules as carriers of hydrophobic bioactive compounds, thus resulting in highly efficient, widely acceptable mode of delivery.

Naturally-occurring and/or pre-formed encapsulation systems include cellualr carriers (e.g., Micro-algae [UTEX 2341, BH], Cyclotella spp. [CYP37], and Baker's yeast) and non-cellualr carriers (e.g., milk fat globules, intra-cellular lipid bodies (e.g., oleosomes), and ragwed pollen).

In some embodiemnts, encapsualtion into an encapsualtion system can be achieved by simple diffusion. In some embodiemnts, encapsualtion into an encapsualtion system can be achieved by facilitated diffusion. Non-limiting examples of faciltiated diffusion include vaccum-based diffusion, and high pressure-based diffusion. In some embodiemnts, encapsualtion into an encapsualtion system can be achieved by both simple diffusion and faciltated diffusion.

In some embodiments, the present disclosure is related to using milk fat globules and oleosomes for encapsulating and stabilizing bioactives. In some embodiments, the present disclosure is related to using milk fat globules and oleosomes for encapsulating and stabilizing bioactives using an approach similar to Stephens vacuum and/or pressure assisted encapsulation. In some embodiments, the encapsualtion would result in milk fat globules and oleosomes with proteins, antioxidants, and enzymes encapsualted therewithin.

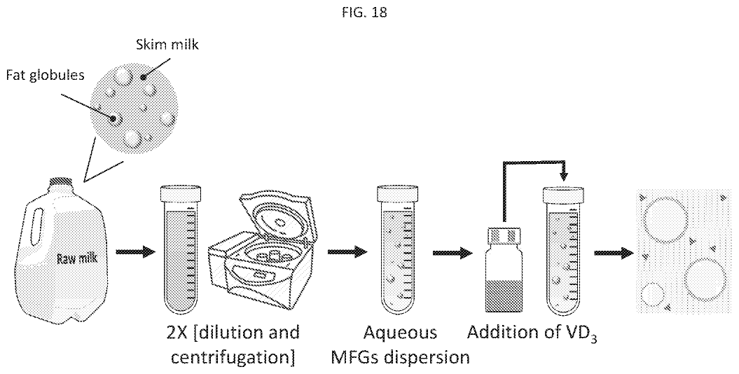

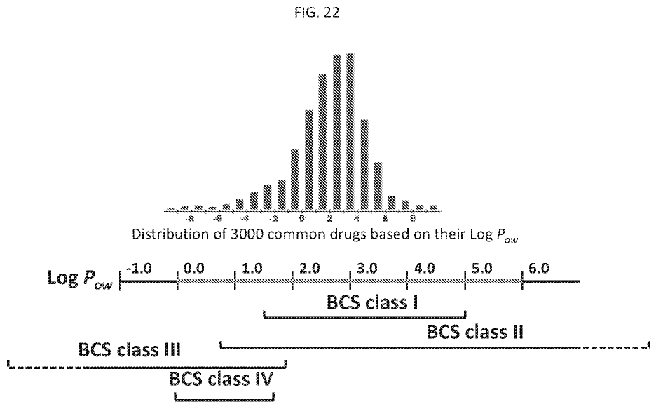

In some embodiments, an embodiment of an encapsualtion process is depicted in FIG. 18. In some embodiments, the process comprises obtianing fat globules from raw milk by dilution and centrifugation, dispersion of milk fat globules in an aquesous medium, addition of the molecule (e.g., Vitamin D3) to be encapsulated to the aqueous dispersion of milk fat globules, and encapsualting the molecule win the milk fat globules. FIG. 22 shows a distribution of 3000 common drugs according to their biopharmaceutical classification based on the biopharmaceutical classification system (BCS) and based on their Log P (Log P_{ow}) value.

In some embodiments, several variables are to be considered for the encapsualtion process. Non-limiting examples of variables include the sourece of the milk fat globules or oelsomes (e.g., bovine milk, ovine milk, nuts, etc.), concentration of the food-grade solvent (e.g, 1% v/v ethanol versus 10% v/v ethanol)

In some embodiments, non-limiting advantages provided by the milk fat globules and oleosomes comprise improved physico-chemical stability including thermal stability, oxidative stability, light stability, and pH stability of the encapsulated molecule. In addition, improved delivery and/or higher effective local concentration of encapsulated molecule to a site of interest (e.g., gut) is provided.

In some embodiments, the at least one compound is fluorescently labeled.

In some embodiments, an intactness of the fraction of the plurality of milk fat globules is determined by labeling the encapsulated compound with a fluorescently-labeled dye. In some embodiments, an intactness of the fraction of the plurality of milk fat globules is determined by determining the intactness of a membrane structure of the plurality of milk fat globules by fluorescence microscopy.

In some embodiments, the fluorescently-labeled dye is can be one or more of Oregon Green™ 488-phosphoethanolamine, or DMEQ-TAD. Other fluorescently-labeled dyes known in the art are also contemplated.

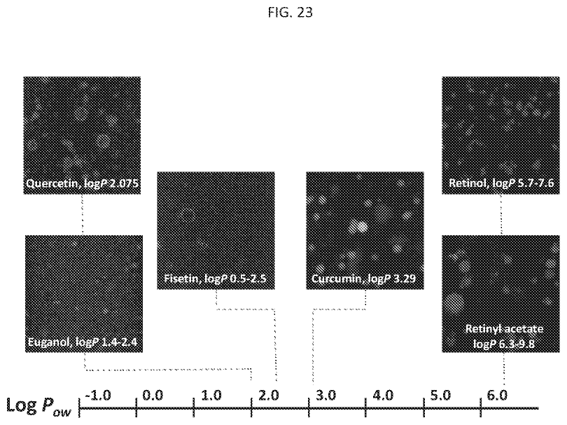

In some embodiments, the at least one compound displays a ring like distribution, peripheral distribution, homogenous distribution, or a combination thereof within the substructure of fraction of the plurality of milk fat globules (FIG. 23).

In some embodiments, the encapsulation is quantified by loading capacity and encapsulation efficiency.

In some embodiments, the milk fat globules can withstand temperatures as low as −80° C. and as high as 80-90° C. In some embodiments, even with freezing of the lipids, no exclusion of the compounds is observed.

In some embodiments, the milk fat globules or oleosomes are “homogenized.” For example, the milk fat globules or oleosomes are of a small size such that they are distributed uniformly throughout another liquid.

In some embodiments of a method for encapsulating at least one compound, the at least one compound is quantified by one or more of fluorescence microscopy, High Performance Liquid Chromatography, Liquid chromatography—mass spectrometry, High Performance Liquid chromatography—mass spectrometry, Gas chromatography—mass spectrometry, ultraviolet—visible spectroscopy or ultraviolet—visible spectrophotometry, ultraviolet—visible-near infrared spectroscopy or ultraviolet—visible spectrophotometry, Raman spectroscopy, and Fourier-transform infrared spectroscopy.

In some embodiments of a method for encapsulating at least one compound, the at least one compound displays a ring like distribution, peripheral distribution, homogenous distribution, or a combination thereof within the substructure of fraction of the plurality of milk fat globules.

In some embodiments of a method for encapsulating at least one compound, the incubating is performed a temperature range of about 4° C. to about 40° C. In some embodiments, the incubating is performed a temperature range of about −10° C. to about 90° C.

In some embodiments of a method for encapsulating at least one compound, the incubating is performed for about 30 sec to about 50 h. In some embodiments, the incubating is performed for about 10 min to about 60 min.

In some embodiments of a method for encapsulating at least one compound, the at least one compound is encapsulated at a concentration range of about 5 μg/gm of milk fat to about 100 μg/gm of milk fat. In some embodiments, the at least one compound is encapsulated at a concentration range of about 0.05 μg/gm of milk fat to about 10,000 μg/gm of milk fat.

In some embodiments of a method for encapsulating at least one compound, the at least one compound is encapsulated at a range of about 10% to about 100% of the dosage requirement per gm of milk fat. In some embodiments, the at least one compound is encapsulated at a range of about 50% to about 100% of the dosage requirement per gm of milk fat.

In some embodiments of a method for encapsulating at least one compound, the at least one compound is provided at a concentration range of about 50 μM to about 2500 μM. In some embodiments, the at least one compound is provided at a concentration range of about 0.5 μM to about 25 mM.

In some embodiments of a method for encapsulating at least one compound, the at least one compound is provided at a dose of about 0.01 mg/kg/day to about 1000 mg/kg/day. In some embodiments, the at least one compound is provided at a dose of about 0.001 mg/kg/day to about 10,000 mg/kg/day. In some embodiments, the at least one compound is provided at a dose of about 0.005 mg/kg/day to about 5000 mg/kg/day.

### Compositions and Pharmaceutical Formulations

In some embodiments, a composition comprises a plurality of milk fat globules or oleosomes, at least one compound, an amount of a pharmaceutically acceptable solvent or food-grade solvent, wherein the at least one compound is encapsulated within a fraction of the plurality of milk fat globules or oleosomes.

In some embodiments, the composition is not whole milk. In some embodiments, the composition is not unprocessed milk. In some embodiments, the composition is nether whole milk nor unprocessed milk.

In some embodiments of a composition, the at least one compound is lipophilic or amphiphilic.

In some embodiments of a composition, the at least one compound is one or more of a bio-active compound, a colorant, or a flavor.

In some embodiments of a composition, the at least one compound is provided in a pharmaceutically acceptable solvent or food-grade solvent. Non-limiting examples of pharmaceutically acceptable solvents or food-grade solvents include acetone, Benzyl Alcohol, 1,3-Butylene Glycol, Carbon Dioxide, Castor Oil, Citric Acid, Esters of Mono- and Di-glycerides, Ethyl Acetate, Ethyl Alcohol (Ethanol), Ethyl alcohol denatured with methanol, Glycerol (Glycerin), Glyceryl diacetate, Glyceryl triacetate (Triacetin), Glyceryl tributyrate (Tributyrin), Hexane, Isopropyl alcohol (Isopropanol), Methyl Alcohol (methanol), Methyl ethyl ketone (2-Butanone), Methylene Chloride (Dichloro-methane), Monoglycerides and diglycerides, Monoglyceride citrate, 2-Nitropropane, 1,2-Propylene glycol (1,2-propanediol), Propylene glycol mono-esters and diesters of fat-forming fatty acids, and Triethyl citrate.

In some embodiments of a composition, the pharmaceutically acceptable solvent or food-grade solvent is selected from the group consisting of an organic solvent, a polar solvent, and a volatile oil.

In some embodiments of a composition, the at least one compound is provided as a micellar composition that fuses with the milk fat globule or oleosome.

In some embodiments of a composition, a concentration of pharmaceutically acceptable solvent or food-grade solvent ranges from about 0.1% v/v to about 10% v/v. In some embodiments, a concentration of pharmaceutically acceptable solvent or food-grade solvent ranges from about 0.5% v/v to about 15% v/v. In some embodiments, a concentration of pharmaceutically acceptable solvent or food-grade solvent ranges from about 1% v/v to about 20% v/v. In some embodiments, a concentration of pharmaceutically acceptable solvent or food-grade solvent ranges from about 0.01% v/v to about 25% v/v.

In some embodiments of a composition, the pharmaceutically acceptable solvent or food-grade solvent is ethanol. In some embodiments, a concentration of ethanol is ≤10% v/v. In some embodiments, a concentration of ethanol ranges from about 0.01% v/v to about 10% v/v.

In some embodiments of a composition, the at least one compound has a Log P value ranging from about 0.5 to about 10. In some embodiments, the at least one compound has a Log P value ranging from about 0.1 to about 10.

In some embodiments of a composition, the at least one compound has a molecular mass ranging from about 50 g/mol to about 5000 g/mol. In some embodiments, the at least one compound has a molecular mass ranging from about 5 g/mol to about 50,000 g/mol.

In some embodiments of a composition, the at least one compound is a food compound is one or more lipid soluble vitamins Non-limiting examples include Vitamin A, Vitamin D, Vitamin K, and their derivatives. In some embodiments of a composition, the at least one compound is one or more flavors. Non-limiting examples include Eugenol, Limonene, Vanillin, Rosemary, Dairy Flavors, and the like. In some embodiments of a composition, the at least one compound is one or more colors. Non-limiting examples include curcumin, annatto extract, capsaicin, and the like. In some embodiments of a composition, the at least one compound is one or more polyphenolic antioxidants. Non-limiting examples include Flavonoids, Anthocynanins, Proanthocyanidins, Curcuminoids, carotenoids, and retinoids. In some embodiments, the at least one compound is fisetin, quercetin hydrate, eugenol, colecalciferol, retinyl acetate, and curcumin.

In some embodiments of a composition, the at least one compound is a pharmaceutical compound selected from the group consisting of HIV drugs, Cardiovascular drugs, antibiotic and antifungals, and oral anti-cancer drugs. Non-limiting examples are shown in Tables 0.1 and 0.2.

In some embodiments of a composition, the at least one compound is encapsulated at a concentration range of about 5 μg/gm of milk fat to about 100 μg/gm of milk fat. In some embodiments, the at least one compound is encapsulated at a concentration range of about 0.05 μg/gm of milk fat to about 10,000 μg/gm of milk fat.

In some embodiments of a composition, the at least one compound is provided at a concentration range of about 50 μM to about 2500 μM. In some embodiments, the at least one compound is provided at a concentration range of about 0.5 μM to about 25 mM.

In some embodiments of a composition, the at least one compound is encapsulated at a range of about 10% to about 100% of the dosage requirement per gm of milk fat. In some embodiments, the at least one compound is encapsulated at a range of about 50% to about 100% of the dosage requirement per gm of milk fat.

In some embodiments of a composition, a size of the fraction of the plurality of milk fat globules or oleosomes comprising the at least one compound ranges from about 5 μm to about 50 μm. In some embodiments, a size of the fraction of the plurality of milk fat globules or oleosomes comprising the at least one compound ranges from about 0.05 μm to about 500 μm. In some embodiments of a composition, a size of the fraction of the plurality of milk fat globules or oleosomes comprising the at least one compound ranges from about 0.1 μm to about 50 μm. In some embodiments of a composition, a size of the fraction of the plurality of milk fat globules or oleosomes comprising the at least one compound ranges from about 0.1 μm to about 10 μm.

In some embodiments of a composition, the composition is for one or more of oral administration, transdermal administration, and topical administration. Other routes of administration are also contemplated. Non-limiting examples include one or more of parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, or intranasal.

In some embodiments of a composition, the milk fat globules or oleosomes are present at a concentration of about 0.1% w/v to about 50% w/v. In some embodiments, the milk fat globules or oleosomes are present at a concentration of about 0.01% w/v to about 25% w/v. In some embodiments of a composition, the milk fat globules or oleosomes are present at a concentration of about 1% w/v to about 90% w/v. In some embodiments of a composition, the milk fat globules or oleosomes are present at a concentration of about 50% w/v to about 95% w/v.

In some embodiments, a composition comprises a plurality of milk fat globules or oleosomes, at least one compound, an amount of a pharmaceutically acceptable solvent or food-grade solvent, wherein the at least one compound is encapsulated within a fraction of the plurality of milk fat globules or oleosomes, and wherein the composition is configured to deliver the compound at a site of interest.

In some embodiments of a composition, the composition is configured to deliver the compound at a site of interest by gradually releasing the compound over a period of time. In some embodiments, the period of time ranges from about 2 h to about 24 h. In some embodiments, the period of time ranges from about 1 min to about 6 h. In some embodiments, the period of time ranges from about 4 h to about 96 h. In some embodiments, the period of time ranges from about 12 h to about 168 h.

In some embodiments of a composition, the composition is configured to deliver the compound at a site of interest by immediately releasing the compound. In some embodiments, immediate release occurs within about 5 sec to about 1 min In some embodiments, immediate release occurs within about 1 sec to about 1 min.

In some embodiments of a composition, the fraction of the plurality of milk fat globules comprising the at least one compound ranges from about 30% to about 100%. In some embodiments, the fraction of the plurality of milk fat globules comprising the at least one compound ranges from about 60% to about 100%. In some embodiments, the fraction of the plurality of milk fat globules comprising the at least one compound ranges from about 90% to about 100%. In some embodiments, the fraction of the plurality of milk fat globules comprising the at least one compound is 100%.

In some embodiments of a composition, the site of interest is one or more of stomach, small intestine, large intestine, liver, skin, oral cavity, and teeth. Other non-limiting examples of sites of interest include skin, brain, kidney, lymph nodes, lymphatic system, blood, cornea, and vitreous humor.

In some embodiments, ≤10% of the encapsulated compound is released in a site that is not a site of interest. In some embodiments, ≤5% of the encapsulated compound is released in a site that is not a site of interest. In some embodiments, ≤1% of the encapsulated compound is released in a site that is not a site of interest. In some embodiments, >90% of the encapsulated compound is released at a site that is a site of interest. In some embodiments, >95% of the encapsulated compound is released at a site that is a site of interest. In some embodiments, >99% of the encapsulated compound is released at a site that is a site of interest. For example, for a compound interested to be delivered to the intestine, ≤20% of the encapsulated compound is released in gastric environment and >80% release in intestinal environment.

In some embodiments of a composition, the at least one compound encapsulated within the plurality of milk fat globules or oleosomes is stabilized against acid degradation. In some embodiments, the at least one compound encapsulated within the plurality of milk fat globules or oleosomes is stabilized against acid degradation at a pH of range about 0.5 to about 5. In some embodiments, the at least one compound encapsulated within the plurality of milk fat globules or oleosomes is stabilized against acid degradation at a pH range of about 0.1 to about 5.

In some embodiments of a composition, the at least one compound encapsulated within the plurality of milk fat globules or oleosomes is stabilized against degradation under alkaline conditions. In some embodiments, the at least one compound encapsulated within the plurality of milk fat globules or oleosomes is stabilized against degradation at a pH range of about 7 to about 10. In some embodiments, the at least one compound encapsulated within the plurality of milk fat globules or oleosomes is stabilized against acid degradation at a pH of about 9 to about 14.

In some embodiments of a composition, the at least one compound encapsulated within the plurality of milk fat globules or oleosomes is stabilized against degradation under both acidic and alkaline conditions.

In some embodiments of a composition, a degradation and/or loss of activity one compound encapsulated within the plurality of milk fat globules or oleosomes under acidic and/or alkaline pH conditions is determined by measuring a degradation product of the at least one compound. In some embodiments, a degradation and/or loss of activity one compound encapsulated within the plurality of milk fat globules or oleosomes under acidic and/or alkaline pH conditions is determined by measuring a degradation product of the at least one compound by Liquid chromatography—mass spectrometry. In some embodiments, a degradation and/or loss of activity one compound encapsulated within the plurality of milk fat globules or oleosomes under acidic and/or alkaline pH conditions is determined by measuring a degradation product of the at least one compound by one or more of fluorescence microscopy, High Performance Liquid Chromatography, Liquid chromatography—mass spectrometry, High Performance Liquid chromatography—mass spectrometry, Gas chromatography—mass spectrometry, ultraviolet—visible spectroscopy or ultraviolet—visible spectrophotometry, ultraviolet—visible-near infrared spectroscopy or ultraviolet—visible spectrophotometry, Raman spectroscopy, and Fourier-transform infrared spectroscopy.

In some embodiments of a composition, a size of the fraction of the plurality of milk fat globules or oleosomes comprising the at least one compound ranges from about 5 μm to about 50 μm. In some embodiments, a size of the fraction of the plurality of milk fat globules or oleosomes comprising the at least one compound ranges from about 0.1 μm to about 50 μm. In some embodiments, a size of the fraction of the plurality of milk fat globules or oleosomes comprising the at least one compound ranges from about 0.1 μm to about 100 μm.

In some embodiments of a composition, the composition is for one or more of oral administration, transdermal administration, and topical administration. Other routes of administration are also contemplated. Non-limiting examples include one or more of parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, or intranasal.

In some embodiments of a composition, about 10% to about 80% of the at least one compound is released from the fraction of the plurality of milk fat globules comprising the at least one compound. In some embodiments, about 50% to about 100% of the at least one compound is released from the fraction of the plurality of milk fat globules comprising the at least one compound. In some embodiments of a composition, about 5% to about 90% of the at least one compound is released from the fraction of the plurality of milk fat globules comprising the at least one compound. In some embodiments of a composition, 100% of the at least one compound is released from the fraction of the plurality of milk fat globules comprising the at least one compound.

In some embodiments of a composition, the at least one compound is provided at a dose of about 0.01 mg/kg/day to about 1000 mg/kg/day. In some embodiments, the at least one compound is provided at a dose of about 0.001 mg/kg/day to about 10,000 mg/kg/day. In some embodiments, the at least one compound is provided at a dose of about 0.005 mg/kg/day to about 5000 mg/kg/day.

In some embodiments, a composition comprises at least one compound encapsulated within a fraction of the plurality of milk fat globules or oleosomes by any of the methods provided herein. a composition comprises at least one compound encapsulated within a fraction of the plurality of milk fat globules or oleosomes by a method comprising providing a plurality of milk fat globules or oleosomes, providing at least one compound, mixing the plurality of milk fat globules or oleosomes with the at least one compound to obtain a mix, incubating the mix to allow the at least one compound to diffuse into at least one substructure of a fraction of the plurality of milk fat globules or oleosomes comprising the at least one compound, thereby encapsulating the at least one compound, wherein the composition is configured to deliver the compound at a site of interest.

In some embodiments of a composition, the composition provides one or more thermal stability, oxidative stability, improved delivery, light stability, and pH stability to the at least one compound.

In some embodiments, the milk fat globules or oleosomes disclosed herein eliminate the need for exogenous antioxidants. In some embodiments, the milk fat globules or oleosomes provide non-thermal based encapsulation suitable for heat labile compounds. In some embodiments, the milk fat globules or oleosomes provide stability during gastric pass. In some embodiments, surface biomolecules on the milk fat globules or oleosomes mediate adhesion and binding to target cells and surfaces.

In some embodiments, the milk fat globules or oleosomes can encapsulate Generally recognized as safe (GRAS) substances. GRAS is a United States Food and Drug Administration (FDA) designation that a chemical or substance added to food is considered safe by experts, and so is exempted from the usual Federal Food, Drug, and Cosmetic Act (FFDCA) food additive tolerance requirements.

In some embodiments, the diversity of molecules that can be encapsulated or bound in the milk fat globules or oleosomes provided herein are unparalleled with other encapsulation systems.

In some embodiments, the ease of the encapsulation process and availability of milk from different animal sources makes this a universal delivery system.

In some embodiments, applications include food supplementation, drug delivery, and food flavors.

In some embodiments of a composition, the composition provides biocompatibility to the at least one compound. In some embodiments, the composition provides extended stability to the at least one compound. In some embodiments, the composition reduces a toxicity of the at least one compound. In some embodiments, the composition provides a controlled release of the at least one compound. In some embodiments, the composition provides a controlled delayed release of the at least one compound. In some embodiments, the composition provides a controlled immediate release of the at least one compound.

In some embodiments, a composition comprises an intact milk fat globule or oleosome, at least one compound, wherein the at least one compound is encapsulated within the intact milk fat globule.

In some embodiments of a composition, the compound comprises at least one of a protein, an antioxidant, and an enzyme. In some embodiments, the compound comprises one or more of a protein, an antioxidant, and an enzyme.

In some embodiments of a composition, the composition is stable during gastric pass.

In some embodiments, a pharmaceutical formulation comprises one or more of the composition provided herein, and a pharmaceutically acceptable carrier.

In some embodiments, pharmaceutical formulations comprises one or more of the composition provided herein, and active ingredients, inactive ingredients, excipients, additives, and/or pharmaceutically acceptable carriers. Examples of additives include natural polymer compounds, inorganic salts, binders, lubricants, disintegrants, surfactants, thickeners, coating agents, pH adjusters, antioxidants, flavoring agents, preservatives, and colorants among others. Examples of other pharmaceutically acceptable carriers include liquid carriers such as water, alcohol, emulsion, and solid carriers such as gel, powder, etc. Standard pharmaceutical formulation techniques and ingredients can be used, such as those disclosed in Remington's The Science and Practice of Pharmacy, 21st Ed., Lippincott Williams & Wilkins (2005), which is hereby incorporated by reference in its entirety.

In some embodiments, pharmaceutical formulations of compositions for intravenous administration comprise excipient and pharmaceutically acceptable carries including one or more of sodium chloride, dextrose, and sterile water. Compositions can comprise, consist of, consist essentially of, aqueous isotonic sterile injection solutions, which can comprise, consist of, consist essentially of, one or more of antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some embodiments, pharmaceutical formulations of compositions for oral administration can be any dosage form that is suitable for oral ingestion, for example, liquid compositions such as elixir, suspension, syrup, emulsion, ampoule, etc., solid compositions such as gel, gum, drop, powder, granule, pill, sugar-coated tablet, film-coated tablet, capsule, package agent, etc. Also contemplated are sustained-release compositions such as gel-coated compositions, multi-coated compositions, localized release compositions.

### Feasibility of Encapsulating Diverse Lipophilic Molecules in Milk Fat Globules

### Background

Demand for high performing encapsulation and delivery systems without exogenous additives or preservatives, as well as reduced environmental impact and cost, has promoted the use of naturally-occurring and pre-formed encapsulation systems (1). These systems are either cell-based or non-cell based carriers that can be isolated from natural products such as food or by products of food production. Examples of such systems include yeast cells, yeast cell wall particles, oleosomes, and pollen shells (2-4). The unique advantages of these encapsulation and delivery systems are high biocompatibility, extended stability, low toxicity, controlled release, and overall high consumer acceptance (5, 6). Additionally, these systems offer a major processing and scale up advantage over their engineered counter parts, as they often do not require extensive modification beyond extraction and isolation from their natural sources.

Conventional encapsulation systems such as emulsions, spray dried particles, and liposomes are typically optimized for the loading of a particular encapsulate or group of encapsulates. Such modifications are achievable via formulation, design, and process adaptations in order to generate maximal compatibility. On the other hand, encapsulation into pre-formed structures requires affinity of encapsulates for the carrier structure to enable partitioning and consequent encapsulation. Partitioning is often achieved using a simple passive diffusion process that may be assisted by an external processing aid such as vacuum pressure (7, 8). Consequently, employing pre-formulated and pre-structured systems for encapsulation necessitates the development of an understanding of the system's limitations in term of the diversity of compounds suitable for encapsulation, which can significantly impact their application potential. To address this issue, this study was aimed at investigating the potential of encapsulating a range of lipophilic compounds with varying degrees of physiochemical properties in the preformed structure of milk fat globules (MFGs).

MFGs are pre-formed lipid structure that originates from the mammary glands. MFGs are micro lipid droplets composed of a lipid core with complex lipid composition, and stabilized by a monolayer lipoproteins membrane. During the secretion process, the droplets are enveloped with the cell bilayer membrane resulting in a tri-layer complex membrane, the milk fat globule membrane (MFGM). Due to the biological nature of MFGM, partitioning of lipophilic molecules from an aqueous environment to MFGs is assumed to be driven by hydrophobic interactions between lipophilic compounds and MFGs interface. Thus, partitioning of hydrophobic molecules and consequent encapsulation can be predicted using systematic study using molecules' physiochemical properties. One such molecular property that is used to predict partitioning potential is the octanol—water partition coefficient; Log P, which is the logarithm of the ratio of the solute concentration in the octanol phase to the solute concentration in the water phase, at a defined temperature. Octanol was selected over other non-polar phases as it simulates the environment of lipid membranes, thus a good representation of the interaction between compounds and membranes. Hence, Log P is an indicator of the affinity of a compound for membranes, and is routinely used to predict in vivo partitioning, transport, and binding of compounds in pharmacology, as well as studying the interaction of lipophilic bioactive compounds with various biological structures (e.g. proteins, or liposomes as representation of biological membranes) (9, 10). Additionally, we examine the potential role the topological polar surface area (TPSA), defined as the sum of surfaces of polar atoms in a molecule. It is used to assess drug absorption (intestinal absorption, blood—brain barrier penetration, and others), and it negatively correlated with hydrophobic interactions (11).

In this study, we examined the molecular and process-related factors that would affect the encapsulation efficiency and loading capacity into MFGs. The study focuses on molecules' partitioning coefficient (log P from 1.5-8) and TPSA (20-127 A²) as measures of hydrophobicity. To avoid potential role of molecular weight, selected compounds were within a relatively narrow range of molecular weight (average 321±47.3 g/mol). The influence of some processing variables such as compound concentration, carrier solvent concentration, as well as time and temperature were also investigated.

In some embodiments, milk fat globules (MFGs) was used as a model encapsulation system. MFGs are composed of a lipid core surrounded by a complex tri-layer lipoprotein membrane. Five lipophilic bioactive compounds with a wide range of hydrophobicity (octanol/water partitioning coefficient Log P 1.5-8) were used to evaluate the versatility of MFGs as an encapsulation system. Partitioning of compounds was first evaluated using fluorescence imaging based on indigenous fluorescence of encapsulated lipophilic compounds. Two distinct patterns were observed, where low Log P compounds, fisetin (approximate average Log P=1.5) and quercetin (approximate average Log P=2.0), exhibited a preferential accumulation in the peripheral of the MFGs, while eugenol, cholecalciferol, and retinyl acetate (approximate average Log P 2.6-8.0) showed uniform distribution of indigenous fluorescence throughout the MFGs structure. The greatest encapsulation efficiency was achieved by retinyl acetate (≥100%, approximate average Log P=8.0) and the lowest encapsulation efficiency was achieved by fisetin (30%). The effect of encapsulation conditions was also evaluated using retinyl acetate as model compound. Increased ethanol concentration, incubation time and temperature positively affected the partitioning and consequent encapsulation of retinyl acetate. Overall, partitioning of lipophilic molecules through the MFGM can be accurately predicted based on compound's Log P. Results highlight the wide range of compounds that can be encapsulated in complex naturally occurring and pre-formed structures.

### Localization of Lipophilic Bioactives in MFGs

The localization of the tested compounds appears in three distinct patterns. First, eugenol, cholecalciferol, and RA that appears to partition throughout the MFGs structure resulting in a uniform distribution of fluorescence signal. Quercetin partitioning into MFGs resulted in heterogeneous distribution of fluorescence signal. In the case of quercetin higher fluorescence signal intensity is localized at the interface of the structure. This could be a result of favored interaction (i.e. thermodynamically favored due to minimization of free energy of the molecule as it transitions from the aqueous phase to the non-polar phase of MFGM) with MFGM components including proteins, and potentially greater affinity for the bilayer region (24). It is possible to assume that prolonged incubation of quercetin with MFGs might result in more homogenous fluorescence signal, as reported in a study by Pawlikowska-Pawlega et al. (2007)⁽²⁵⁾. In this reported study human skin fibroblasts cells were incubated with quercetin and the fluorescence signal of quercetin exhibited a time-dependent distribution. With extended incubation, (≥1h) the fluorescence signal spread from the membrane to the intracellular compartments of the cell, resulting in relatively homogenous distribution of the fluorescence signal throughout the cell.

Fisetin exhibited limited partitioning into MFGs, forming a ring at the peripheral of the MFG structure. This is potentially linked to its relatively high hydrophilicity (the lowest Log P value among the tested compounds). Fisetin preferential accumulation in the lipid bilayer has been reported in prior studies focused on interactions of fisetin with liposomes and its application as a potential membrane probe (26-28).

Localization of quercetin and fisetin in the MFGM bilayer encourages the use of these compounds as sacrificial antioxidants to protect less stable compounds encapsulated in MFGs core by neutralizing free radical and retarding lipid oxidation (10, 29). Such approach would mimic the natural design of MFGM that contains relatively high content of α-tocopherol (30). As an endogenous antioxidant, α-tocopherol role is assumed to be prevent the initiation of oxidation processes of the unsaturated membrane lipids (30).

### Effect of Compounds' Physiochemical Properties on Encapsulation in MFGs

The study evaluated the influence of relative hydrophobicity on partitioning of the test compounds into MFGs. Tables 2.A and 2.B show test compounds and their LC and % EE, respectively. Compounds are organized in order of average Log P values (experimental and/or theoretical). We note that the EE % (Table 2.B) increases with an increase in Log P value. The % EE was approximately 30% for fisetin (approximate Log P=1.5), and increased to 50% for quercetin (approximate Log P=2.0), and further to approximately 98% for eugenol (approximate Log P=2.6). However, cholecalciferol (approximate Log P=7.5) resulted in a maximal EE % of only 69%, followed by RA (approximate Log P=8.0) reaching an EE % of ≥100%. These results partially deviate from the theoretical expectation, requiring the examination of other factors such as those related to molecule's structural and physiochemical properties, carrier interfacial properties, bulk phase properties, and environmental/processing conditions.

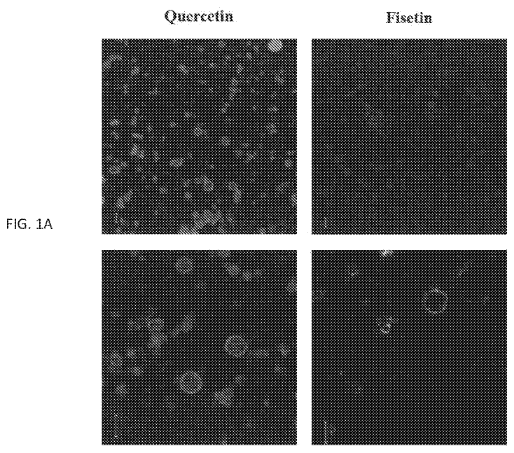

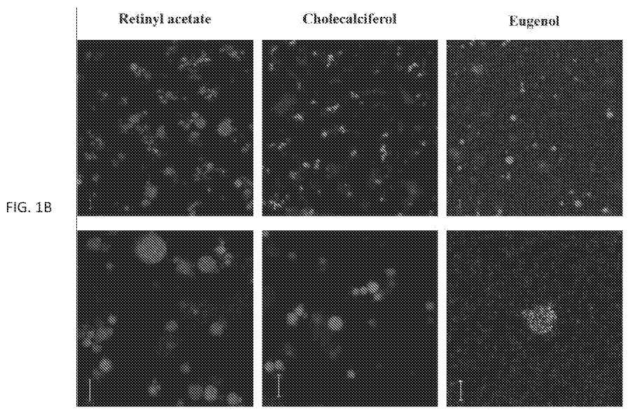

One important molecular feature is the topological polar surface area (TPSA), defined as the sum of surfaces of polar atoms in a molecule. It is used to assess drug absorption (intestinal absorption, blood—brain barrier penetration, and others), and it is negatively correlated with hydrophobic interactions (11). The TPSA values for the test compounds in this study are as follow (A²): fisetin=107⁽¹⁴⁾, quercetin=127⁽³⁴⁾, eugenol=29.5⁽³⁵⁾, cholecalciferol=20.2⁽³⁶⁾, and RA=26.3⁽²¹⁾. With the fisetin and quercetin TPSA (FIG. 1A) being markedly higher than eugenol, cholecalciferol, and RA (FIG. 1B), hence supporting their localization pattern , but does not fully explain the overall trend observed for maximum EE % values (Table 2B)

Additionally, specific modes of interaction between test compounds and MFGs might results in deviation in the partitioning and encapsulation from ideal behavior. For instance, MFGM proteins might play a role in binding lipophilic molecules, thus influencing the EE % and LC, by either allowing greater concentrations to associate with MFGs, or preventing additional molecules from accessing MFGM interior and further diffusing into MFGs core. Other potential factors could be the solubility of these compounds in MFGs core lipids. It is also important to consider the effect of the methodology used for extraction and quantification of encapsulated compounds from MFG matrix. These methods can also potentially bias the results related to the EE and LC of selected compounds in MFGs.

Similar to this study, some of the prior studies have also evaluated partitioning of lipophilic compounds through bilayer membranes of various complexities and compositions (31-33).

### Effect of Carrier Solvent Concentration on the Encapsulation of Retinyl Acetate in MFGs

The effect of ethanol on biological membranes is well documented in the literature. Ethanol causes expansion of the lipid bilayer, consequently increases the membrane fluidity and permeability (37-39). The results show that increasing the ethanol concentration beyond 4% v/v causes little increase in LC and EE %. This could be attributed to increase in polarity of MFGM interface due to association with ethanol, reducing the hydrophobic affinity of RA to MFGM interface (38).

### Effect of Incubation Conditions on the Encapsuation of Retinyl Acetate in MFGs

Limited work is available on the thermal behavior of MFGM due to its complex composition and structure. Murthy et al. (2016)⁽⁴⁰⁾ conducted a study using reconstitiated bi-layer from MFGM lipid extract. This prior study evaluated this reconstituted bi-layerusing variety of methods including differential scanning calorimetry and atomic force microscopy. The results of the study showed that the phase transition of MFGM polar lipids occur at approximately 40° C. In the temperature range between 20-40° C., the solid ordered (s₀) and the liquid-disordered (l_{d}) phases coexist. The s₀ is present in the form of micro-domains in the hydration layer of the bilayer and is characteristically rich in high melting point lipids such as sphingomyelin. These domains are surrounded by id continues phase rich in unsaturated polar lipids (i.e. PE, PS, PI and PC). As the temperature decreases from 40° C. to 20° C., the proportion of s₀ phase increases suggesting compactness of the lipid molecular area as a result of ordering of the hydrocarbon chains with a decrease of the lateral mobility of the high melting point polar lipids (40, 41). Similar results were reported by Zou et al. (2015)⁽⁴²⁾, stating a decrease in the number and size of the ordered domains with increasing temperature (from 4° C. to 37° C.), where the level of membrane heterogeneity significantly increases at lower temperature (i.e. 4° C.).

Results obtained in this study regarding the influence of incubation temperature on the partitioning and encapsulation of RA in MFGs (Table 5), show increase in the LC and EE % with increased temperature (from 4° C. to 40° C.). This is likely due increased fluidity and consequently permeability of the MFGM due to temperature-induced phase transition as the temperature increases from 4° C. to 40° C. (41, 43).

In addition to the thermally induced changes in the physical properties of MFGM, the MFGs lipid core is expected to undergo some changes in its physical status in the temperature range tested in this study. Lopez et al. (2002)⁽⁴⁴⁾ have reported that milk triglycerides in MFGs (cream) undergo crystallization starting 19° C., therefore MFGs lipid core is partially crystalized at room temperature. Hence, in the test conditions of 4° C. and 22° C., partial crystallization of milk fat could contribute to reduced solubility of RA, supporting the lower LC and EE % values. Furthermore, milk fat have been reported to be completely melt at temperatures above 41° C., therefore the high loading at 40° C., followed by crystallization at room temperature conditions used in the extraction steps may explain EE % values that exceed 100% (45).

Furthermore, the thermal effect could be related to correlation between temperature and the rate of diffusion. Increasing temperature increases the kinetic energy of a molecule, and consequently the rate of diffusion through the aqueous and membrane phases.

Lastly, we conclude that incubation of RA with MFGs beyond 40 min does not cause any significant increase in the LC or EE %. This is likely due to reaching maximal solubility in milk fat.

### EXAMPLES

The following examples are non-limiting and other variants within the scope of the art also contemplated.

### Example 1—Materials

Raw whole bovine milk was procured from local markets (Organic Pastures, Fresno, Calif., U.S.A.). Absolute ethanol was obtained from Koptec (King of Prussia, PA, U.S.A.). Quercetin hydrate (849061-97-8), curcumin (CAS 458-37-7, C1386), retinyl acetate (127-47-9), fisetin (345909-34-4), cholecalciferol (67-97-0), eugenol (97-53-0), methanol, chloroform, and sodium chloride were purchased from Sigma-Aldrich (St. Louis, Mo., U.S.A.). Fluorescence labeling agent DMEQ-TAD (4-[2-(6,7-dimethoxy-4-methyl-3-oxo-3,4-dihydroquinoxalyl) ethyl]-1, 2,4-triazoline-3,5-dione was purchased from Abcam (Cambridge, Mass,, U.S.A).

### Example 2—Isolation of Milk Fat Globule from Raw Milk

Raw milk was procured from a local market and stored at 4° C. Milk was used for up to 4 days post purchase. MFGs were separated from raw milk using centrifugal separation following a modified version of the method by Gallier et al. (2010) (12). First, raw milk was diluted with Milli-Q water at 9:1 water to milk, and the solution was centrifuged at 3,000×g for 5 min using a fixed angle centrifuge rotor. Cream was then skimmed off the sides of the centrifuged tubes after discarding the aqueous phase. In order to remove more of the milk serum components, cream was reconstituted in water to reach 4% w/v, and subjected to another step of centrifugation at 3,000×g for 5 min Cream was collected as detailed prior, then reconstituted to 20% w/v in Milli-Q water. All separation steps were completed at room temperature.

### Example 3—Multi-Photon Fluorescence Microscopy of Compounds Encapsulated in Milk Fat Globules

Bioactive compounds were added to MFGs as described in Example 4 at 250 μM concentration and allowed to incubate for 30 min at room temperature. MFGs were then recovered from the aqueous solution via centrifugation as described in Example 5. Cholecalciferol was fluorescently labeled prior to encapsulation.

For imaging purposes, a small quantity of cream was suspended in mixture of water and glycerol to increase the viscosity of the medium and reduce the rate of motion of MFGs particles that results in blurred images. Fluorescence images were collected using Leica TCS SP8 multi-photon microscope (Buffalo Grove, Ill., U.S.A.) equipped with 40×/1.10 HC PL IRAPO water objective. Excitation was achieved using tunable Mai tai deep see laser (690 to 1090 nm) set at resonant mode for high speed scanning, emission was collected on Hyd-RLD 2 detector. Excitation and emissions are noted in Table 1.

### Example 4—Encapsulation of Hydrophobic Compounds into Milk Fat Globules

Encapsulation of test compounds in MFGs was accomplished by mixing ethanolic stock solution of a compound with 20% w/v cream suspended in water. The mixture was gently inverted several times to mix, then allowed in incubate in the dark.

To test the effect of compounds concentration, 50-2500 μM of each compound were added in the presence of 10%v/v ethanol, and the system was incubated for 30 min at room temperature. To assess the effect of ethanol concentration, retinyl acetate (RA) was added at a fixed concentration (250 μM) to 20% w/v cream suspended in water in the presence of 1-10% v/v of ethanol and was allowed to incubate at room temperature for 30 min.

The effect of incubation time was assessed by adding retinyl acetate (RA) was added at a fixed concentration (250 μM) to 20% w/v cream suspended in water in the presence of 1% v/v of ethanol and was allowed to incubate at room temperature for 10,20,30,40,50, or 60 min. To test the effect of incubation temperature on the partitioning and encapsulation yield and efficiency, RA was added at 250 μM concentration and was allowed to incubate with MFGs for 30 min at 4, 22, and 40° C.

All experimental procedures were completed in triplicates (using independent milk samples) and reported as averages ±standard deviation. Statistical significance of the independent effect of each of the tested variables was determined using one-way ANOVA followed by Tukey's post hoc test.

### Example 5—Extraction and Quantification of Encapsulated Compounds

To quantify the amount of compound encapsulated in MFGs, cream was separated from ethanol-containing aqueous phase via centrifugal separation at 3,000 x g for 5 min. Cream was skimmed and added to methanol at 50 mg/mL and vortexed vigorously for 1 min, then centrifuged at 16,000×g for 10 min. Top layer of methanol was collected and absorbance was measured at appropriate wavelength for each compound. An absorbance blank was prepared following the same encapsulation and separation steps in the absence of compound. λₘₐₓ in methanol (nm) for each compound is reported in Table 1.

### Example 6—Determination of Fat Content of Raw Cream

Cream (20% w/v in Milli-Q water) that was separated following the steps in Example 2 was further separated via centrifugation at 3,000×g for 5 min. Fat content in cream was measured using a modified Folch method (13). An additional washing step was incorporated where the chloroform/water interface was washed with 1:1 water/methanol solution.

### Example 7—Statistical Analysis

Statistical analysis was carried out using Microsoft Excel 2010 (Microsoft Inc., Bellevue, Wash., U.S.A.).

TABLE 1
Lipophilic bioactive compounds tested in this
study and their physiochemical properties.

| | Water/octanol | | λₘₐₓ in | Multi-photon |
|---|---|---|---|---|
| Bioactive | partitioning | | methanol | excitation/ |
| compound | coefficient, Log P* | Bioactivity | (nm)** | emission (nm) |
| Fisetin | 0.464-2.52 ^{(14, 15)} | Coloring agent, | 362 | 760/580 |
| C₁₅H₁₀O₆ | | anti-inflammatory | | |
| Phenolic | | | | |
| compound | | | | |
| (flavonol) | | | | |
| Quercetin | 1.82-2.075 ^{(16, 17)} | Antioxidant, | 372 | 740/580 |
| hydrate | | anti-inflammatory | | |
| C₁₅H₁₀O₇ | | | | |
| Phenolic | | | | |
| compound | | | | |
| (flavonol) | | | | |
| Eugenol | 2.61-2.66 ⁽¹⁸⁾ | Antibacterial, | 282 | 720/580 |
| C₁₀H₁₂O | | antifungal | | |
| Phenylpropene | | | | |
| Cholecalciferol | 7.13-7.98 ^{(19, 20)} | Antioxidant | 265 | 740/580 |
| C₂₇H₄₄O | | | | |
| Steroid hormone | | | | |
| Retinyl acetate | 6.3-9.81 ^{(21, 23)} | Antineoplastic, | 325 | 730/580 |
| C₂₂H₃₂O₂ | | chemopreventive | | |
| Fatty acid ester | | | | |
| form of retinol | | | | |

*Measured or estimated. Please refer to reference/s for more information
**Determined experimentally

### Example 8—Localization of Encapsulated Lipophilic Compounds in MFGS using Fluorescent Imaging

The results in FIGS. 1A and 1B show a cross section of MFGs where the fluorescent signal is a result of indigenous fluorescence of encapsulated lipophilic compounds. The goal of these imaging measurements was to validate the partitioning of lipophilic bioactives into MFGs structure and to assess the influence of compounds relative hydrophobicity and structural properties on the localization in MFGs, using indigenous fluorescence of each compound.

The first row in FIGS. 1A and 1B shows indigenous fluorescence of fisetin in MFGs that appears in a distinct ring structure. The second row shows quercetin, which exhibits greater density of fluorescent signal in the peripheral of MFGs structures. On the other hand, eugenol, cholecalciferol, and retinyl acetate exhibit uniform indigenous fluorescence signal distribution in the MFGs structure.

### Example 9—Effect of Compound Concentration on Encapsulation of Lipophilic Compounds into MFGs

The goals of these set of experiments were to quantify the loading capacity (LC) and encapsulation efficiency (EE %) of diverse compounds in MFGs as affected by compound's concentration. Table 2 shows the LC of each of the test compounds as a function of increasing compound concentration in the continuous aqueous phase under fixed ethanol content (10% v/v) and incubation conditions (30 min at room temperature). The following maximal values were achieved for each compound: 9.77 μg of fisetin, 12.97 μg of quercetin, 39.65 μg of eugenol, 22.12 μg of cholecalciferol, and 74.15 μg RA per gram of milk fat. Levels of significance are denoted in Table 2.A. Note that LC of eugenol were not calculated at addition level below 150 μM due to low absorbance value recorded spectrophotometrically (i.e. <0.100).

Table 2 shows the corresponding EE % of LC values reported in Table 2.A. The overall greatest encapsulation efficiency was achieved by RA (≥100%). EE % values of greater of 100%, this is likely due to crystallization of the compound due to poor solubility in the aqueous phase, which affects the extraction step.

TABLE 2

| Concentration | | | | | |
|---|---|---|---|---|---|
| of added | % Encapsulation efficiency | | | | |
| compound | | | | | Retinyl |
| (μM) | Fisetin | Quercetin | Eugenol | Cholecalciferol | acetate |
| 50 | 28.64 ± 5.20 | 25.27 ± 3.47 | — | 68.60 ± 6.30 | 107.46 ± 6.29 |
| 100 | 29.16 ± 1.50 | 45.35 ± 8.53 * | — | 69.58 ± 6.44 | 100.56 ± 3.83 |
| 150 | 30.55 ± 3.76 | 43.78 ± 4.16 * | 98.05 ± 4.10 | 67.62 ± 3.67 | 111.73 ± 6.32 |
| 200 | 29.83 ± 2.47 | 50.09 ± 3.71 * | 95.43 ± 7.46 | 63.59 ± 1.43 | 110.33 ± 3.15 |
| 250 | 28.41 ± 1.56 | 48.95 ± 3.47 * | 86.64 ± 7.23 | 49.84 ± 3.74 * | 105.92 ± 4.41 |
| 500 | 15.89 ± 1.42 *† | 49.00 ± 1.17 * | 85.14 ± 6.33 | 45.25 ± 3.00 *† | 76.40 ± 9.65 *‡ |
| 1000 | 15.16 ± 1.46 *† | 12.91 ± 1.78 † | 80.94 ± 2.42 *† | 24.77 ± 0.84 *† | 86.09 ± 12.48 ‡ |
| 1500 | 7.57 ± 0.43 * | 18.38 ± 9.27 † | 82.09 ± 134 * | 28.92 ± 3.43 *† | 82.14 ± 15.94 *‡ |
| 2000 | 14.26 ± 2.77 * | 17.92 ± 4.31 † | 80.86 ± 2.58 *† | 19.66 ± 1.05 *† | 76.89 ± 9.88 *‡ |
| 2500 | 4.49 ± 1.08 *‡ | 13.70 ± 1.30 † | 80.71 ± 3.39 *† | 19.22 ± 2.88 *† | 75.45 ± 5.19 *†‡ |

All compounds exhibited a trend of increase in EE % followed by decrease and/or plateau. For instance, fisetin reached its highest EE % when 150 μM were present in the aqueous phase, EE % significantly decreased when ≥500 μM was added. Maximal EE % was also achieved at 150 μM of added quercetin, RA, and eugenol. Cholecalciferol reached its highest EE % at 100 μM and starts to significantly decrease >250 μM. Thus it appears that a small concentration of compound (low proportion of compound to MFGs) is required to drive partitioning into MFGs.

### Example 10—Effect of Carrier Solvent Concentration on Encapsulation of Retinyl Acetate in MFGs

Due to the high EE % of RA in MFGs, and its physiological significance as a vitamin, the compounds was selected as a model compound to test the effects of encapsulation processes conditions. Table 3 shows the loading capacity and EE % of RA as a function of ethanol concentration in the aqueous phase. Increasing the concentration of ethanol from 1 to 10% v/v resulted in statistically significant (p<0.05) increase in the loading capacity of RA in MFGs, corresponding to an increase from 7.6 to 10.57 μg of RA/g of milk fat, respectively. Similarly, the EE % increased with increased ethanol concentration (78-107%), reaching a maximum of approximately 107% at ethanol concentration greater than 4% v/v. Using Tukey's post hoc analysis, 4-10% v/v ethanol significantly increase both the LC and EE % compared to 1 and 2% v/v ethanol.

TABLE 3

| Ethanol | Loading capacity | |
|---|---|---|
| concentration | (μg retinyl acetate/ | % Encapsulation |
| (v/v %) | g of milk fat) | efficiency |
| 1 | 7.65 ± 0.54 | 77.86 ± 5.54 |
| 2 | 8.20 ± 1.15 | 83.40 ± 11.73 |
| 4 | 9.95 ± 0.47 * | 101.24 ± 4.76 * |
| 6 | 10.51 ± 1.07 *† | 106.90 ± 10.93 *† |
| 8 | 10.58 ± 0.63 *† | 107.65 ± 6.42 *† |
| 10 | 10.57 ± 0.51 *† | 107.53 ± 5.15 *† |

### Example 11—Effect of Incubation Conditions on the on Encapsulation of Retinyl Acetate in MFGs

Incubation time of MFGs with RA was varied between 10 and 60 min at 10 min intervals. A statistically significant increase in the loading capacity of RA in MFGs was achieved with increased incubation time (p<<0.05) reaching 9.28 μg RA/g of milk fat at 60 min incubation, Table 4. Correspondingly, % EE increased with increased incubation time. Levels of significance are reported in Table 4.

TABLE 4

| | Loading capacity | |
|---|---|---|
| | (μg retinyl acetate/ | % Encapsulation |
| Time (min) | g of milk fat) | efficiency |
| 10 | 5.11 ± 0.46 | 52.02 ± 4.66 |
| 20 | 6.45 ± 0.63 * | 65.66 ± 6.40 * |
| 30 | 7.48 ± 0.09 * | 76.11 ± 0.89 * |
| 40 | 7.76 ± 0.50 *† | 78.98 ± 5.13 *† |
| 50 | 8.78 ± 0.46 *†‡ | 89.38 ± 4.66 *†‡ |
| 60 | 9.28 ± 0.22 *†‡¥ | 94.38 ± 2.25 *†‡¥ |

Table 5 shows the effect of incubation temperature on encapsulation of RA into MFGs at fixed time (30 min), concentration (250 μM), and ethanol content (1% v/v). A statistically significant increase (p<<0.05) is achieved with increasing the temperature. LC reached 12.23 μg RA/g of milk fat after 30 min incubation at 40 ° C. with a corresponding 124.42% EE.

TABLE 5

| | Loading capacity | |
|---|---|---|
| | (μg retinyl acetate/ | % Encapsulation |
| Temperature ° C. | g of milk fat) | efficiency |
| 4 | 5.26 ± 0.39 ^{a} | 53.52 ± 3.97 ^{a} |
| 22 | 7.08 ± 0.38 ^{b} | 72.07 ± 3.85 ^{b} |
| 40 | 12.23 ± 0.64 ^{c} | 124.42 ± 6.52 ^{c} |

### Summary

The results of this study show that MFGs are a suitable encapsulation system for a wide range of lipophilic bioactive compounds with varying degrees of lipophilicity. This feature is likely related to the compositional complexity of the MFGs offering span of physiochemical properties. We conclude that the partitioning of compounds through the MFGM can be effectively enhanced by increasing the concentration of the carrier solvent, and elevating the temperature to/above that of the MFGM's s₀ to l_{d} transition temperature. Overall, the partitioning behavior through the MFGM can be adequately predicted using compound's octanol/water partitioning coefficient. Nevertheless, there is a need to further investigate the underlying molecular properties of a wider range of molecules and form a detailed understanding of specific molecular interaction between partitioning compounds and the various MFGM constituent is essential to create a better predictive model of encapsulate partitioning and localization.

### REFERENCES

All references herein are incorporated by reference in their entireties.

1. Tan S, Wu T, Zhang D, Zhang Z. 2015. Cell or cell membrane-based drug delivery systems. Theranostics 5:863.

2. Nguyen T-T, Phan-Thi H, Pham-Hoang B-N, Ho P-T, Tran TTT, Wache Y. 2018. Encapsulation of Hibiscus sabdariffa L. anthocyanins as natural colours in yeast. Food Research International 107:275-280.

3. Socaciu C, Mihis C, Trif M, Diehl H. 2007, p 6-8. Symposium on Bioencapsulation.

4. Alshehri S M, Al-Lohedan H A, Chaudhary A A, Al-Farraj E, Alhokbany N, Issa Z, Alhousine S, Ahamad T. 2016. Delivery of ibuprofen by natural macroporous sporopollenin exine capsules extracted from Phoenix dactylifera L. European Journal of Pharmaceutical Sciences 88:158-165.

5. Hamidi M, Zarrin A, Foroozesh M, Mohammadi-Samani S. 2007. Applications of carrier erythrocytes in delivery of biopharmaceuticals. Journal of controlled release 118:145-160.

6. Rossi L, Serafini S, Pierige F, Antonelli A, Cerasi A, Fraternale A, Chiarantini L, Magnani M. 2005. Erythrocyte-based drug delivery. Expert opinion on drug delivery 2:311-322.

7. Young S, Dea S, Nitin N. 2017. Vacuum facilitated infusion of bioactives into yeast microcarriers: Evaluation of a novel encapsulation approach. Food Research International 100:100-112.

8. Diego-Taboada A, Beckett S T, Atkin S L, Mackenzie G. 2014. Hollow pollen shells to enhance drug delivery. Pharmaceutics 6:80-96.

9. Leo AJ. 1993. Calculating log Poct from structures. Chemical Reviews 93:1281-1306.

10. Yang B, Kotani A, Arai K, Kusu F. 2001. Estimation of the antioxidant activities of flavonoids from their oxidation potentials. Analytical sciences 17:599-604.

11. Xiao J, Chen T, Cao H, Chen L, Yang F. 2011. Molecular property-affinity relationship of flavanoids and flavonoids for HSA in vitro. Molecular nutrition & food research 55:310-317.

12. Gallier S, Gragson D, Jimenez-Flores R, Everett D. 2010. Using confocal laser scanning microscopy to probe the milk fat globule membrane and associated proteins. Journal of agricultural and food chemistry 58:4250-4257.

13. Folch J, Lees M, Sloane-Stanley G. 1957. A simple method for the isolation and purification of total lipids from animal tissues. Journal of Biological Chemistry 226:497-509.

14. , posting date. Fisetin 528-48-3 I DTXSID4022317 [Online.]

15. , posting date. Fisetin. [Online.]

16. Rothwell J A, Day A J, Morgan M R. 2005. Experimental determination of octanol—water partition coefficients of quercetin and related flavonoids. Journal of agricultural and food chemistry 53:4355-4360.

17. Sutherland I A, Fisher D. 2009. Role of counter-current chromatography in the modernisation of Chinese herbal medicines. Journal of Chromatography A 1216:740-753.

18. , posting date. logP for Euganol. [Online.]

19. vitamin D—Calculator Plugins were used for structure property prediction and calculation, Marvin n.n.n (insert version number), 201n (insert year of version release), ChemAxon (http://www.chemaxon.com).

20. Tetko IV, Gasteiger J, Todeschini R, Mauri A, Livingstone D, Ertl P, Palyulin V A, Radchenko E V, Zefirov N S, Makarenko A S. 2005. Virtual computational chemistry laboratory—design and description. Journal of computer-aided molecular design 19:453-463.

21. , posting date. Retinyl acetate. [Online.]

22. , posting date. Retinol acetate 127-47-9 I DTXSID6021240 [Online.]

23. Bernauer U, Bodin L, Celleno L, Chaudhry Q, Coenraads PJ, Dusinska M, Duus-Johansen J, Ezendam J, Gaffet E, Galli C L. 2016. Scientific Committee on Consumer Safety SCCS OPINION ON Vitamin A (Retinol, Retinyl Acetate, Retinyl Palmitate).

24. Kośinová Pn, Berka K, Wykes M, Otyepka M, Trouillas P. 2012. Positioning of antioxidant quercetin and its metabolites in lipid bilayer membranes: implication for their lipid-peroxidation inhibition. The Journal of Physical Chemistry B 116:1309-1318.

25. Pawlikowska-Pawlega B, Gruszecki W I, Misiak L, Paduch R, Piersiak T, Zarzyka B, Pawelec J, Gawron A. 2007. Modification of membranes by quercetin, a naturally occurring flavonoid, via its incorporation in the polar head group. Biochimica et Biophysica Acta (BBA)-Biomembranes 1768:2195-2204.

26. Guharay J, Dennison S M, Sengupta P K. 1999. Influence of different environments on the excited-state proton transfer and dual fluorescence of fisetin. Spectrochimica Acta Part A: Molecular and Biomolecular Spectroscopy 55:1091-1099.

27. Mohapatra M, Mishra A K. 2011. Photophysical behavior of fisetin in dimyristoylphosphatidylcholine liposome membrane. The Journal of Physical Chemistry B 115:9962-9970.

28. Sengupta B, Banerjee A, Sengupta PK. 2004. Investigations on the binding and antioxidant properties of the plant flavonoid fisetin in model biomembranes. FEBS letters 570:77-81.

29. Bricarello D A, Prada M J, Nitin N. 2012. Physical and chemical modifications of lipid structures to inhibit permeation of free radicals in a supported lipid membrane model. Soft Matter 8:11144.

30. Jensen S K, Nielsen K N. 1996. Tocopherols, retinol, (3-carotene and fatty acids in fat globule membrane and fat globule core in cows' milk. Journal of Dairy Research 63:565-574.

31. Nikaido H. 1976. Outer membrane of Salmonella typhimurium: transmembrane diffusion of some hydrophobic substances. Biochimica et Biophysica Acta (BBA)-Biomembranes 433:118-132.

32. Sha'Afi R, Gary-Bobo C, Solomon A. 1971. Permeability of red cell membranes to small hydrophilic and lipophilic solutes. The Journal of general physiology 58:238-258.

33. Levin V A. 1980. Relationship of octanol/water partition coefficient and molecular weight to rat brain capillary permeability. Journal of medicinal chemistry 23:682-684.

34. posting date. Quercetin. [Online.]

35. posting date. eugenol pubchem. [Online.]

36. posting date. Vitmian D pubchem. [Online.]

37. Komatsu H, Okada S. 1997. Effects of ethanol on permeability of phosphatidylcholine/cholesterol mixed liposomal membranes. Chemistry and Physics of Lipids 85:67-74.

38. Ingram L O. 1989. Ethanol tolerance in bacteria. Critical Reviews in Biotechnology 9:305-319.

39. Lieb W, Stein W. 1969. Biological membranes behave as non-porous polymeric sheets with respect to the diffusion of non-electrolytes. Nature 224:240.

40. Murthy A V R, Guyomarc'h F, Lopez C. 2016. The temperature-dependent physical state of polar lipids and their miscibility impact the topography and mechanical properties of bilayer models of the milk fat globule membrane. Biochimica et Biophysica Acta (BBA)-Biomembranes 1858:2181-2190.

41. Shimanouchi T, Ishii H, Yoshimoto N, Umakoshi H, Kuboi R. 2009. Calcein permeation across phosphatidylcholine bilayer membrane: effects of membrane fluidity, liposome size, and immobilization. Colloids and Surfaces B: Biointerfaces 73:156-160.

42. Zou X, Guo Z, Jin Q, Huang J, Cheong L, Xu X, Wang X. 2015. Composition and microstructure of colostrum and mature bovine milk fat globule membrane. Food chemistry 185:362-370.

43. Gennis RB. 2013. Biomembranes: molecular structure and function. Springer Science & Business Media.

44. Lopez C, Bourgaux C, Lesieur P, Bernadou S, Keller G, Ollivon M. 2002. Thermal and Structural Behavior of Milk Fat. Journal of Colloid and Interface Science 254:64-78.

45. Hu J, Sari O, Eicher S, Rakotozanakajy A R. 2009. Determination of specific heat of milk at different fat content between 1 C and 59 C using micro DSC. Journal of food engineering 90:395-399.

### Milk Fat Globules, a Novel Carrier for Delivery of Exogenous Cholecalciferol

### Background

Vitamin D₃ (cholecalciferol) is a fat-soluble compound present in the blood in the form of 25-hydroxyvitamin D (calcifediol), and acts as a pro-hormone. The primary function of vitamin D (VD) is to control absorption, transport, and deposition of calcium and other minerals essential for bone health and muscle function (Turner, Anderson, & Morris, 2012).

VD deficiency is a worldwide public health problem (Mithal et al., 2009; Palacios & Gonzalez, 2014; Wahl et al., 2012). Deficiency of VD is defined as ≤20 ng/mL blood levels, causing inefficiency in absorption of calcium and other minerals, which compromises bone health and muscle function. Prolonged and extreme deficiency of VD can result in rickets in children and osteomalacia in adults (Calvo & Whiting, 2013; Holick et al., 2011). One of the major causes of VD deficiency is limited dietary sources of VD (Hossein-nezhad & Holick, 2013). Few food items are naturally rich in VD, this includes fatty fish and beef liver that are rich in VD3, and food items such as baker's yeast and mushrooms that are treated with ultraviolet-A light to induce generation of VD₂ (ergocalciferol) from ergosterol. While both forms of VD are used in food fortification worldwide, VD₃ is more commonly used, potentially due to its bioefficacy and relatively higher chemical stability in powder form (Grady & Thakker, 1980; Holick et al., 2011; Tripkovic et al., 2012).

In addition to indigenous VD sources, fortified foods are a major source of VD in the western diet, accounting up to approximately 60% of the consumed vitamin (Calvo & Whiting, 2013; Holick et al., 2011). In the United States of America, fortification with VD is optional under the Food and Drug Administration regulations; currently only milk and ready-to-eat cereals are regularly fortified. Varying national policies are in place amongst European Union (EU) countries, with a few countries such as Finland, Sweden, and the United Kingdom having mandatory fortification requirements for some dairy products such as milk and infant formula, as well as margarine(Spiro & Buttriss, 2014). A few Southeast Asian countries such as Malaysia, the Philippines, and Indonesia require fortification of some dairy products and margarine (Darnton-Hill & Nalubola, 2002).

Sources of VD₃ include natural sources (e.g., fatty fish and fish liver oils, Beef liver, Cheese and egg yolk, UV treated mushroom and yeast, and fortified foods (e.g., Liquid formulations such as milk (100 IU/cup in the U.S.A) and juice, and Dry formulations such as ready-to-eat breakfast cereals).

Despite food fortification efforts, recent studies indicate that in the U.S.A. and Canada, the mean dietary intake of VD is less than 400 IU, well below the recommended dietary allowances (RDAs) of 600-800 IU per day (1IU=0.025 μg for both ergo- and chol-calciferol) (Calvo & Whiting, 2013). This could be a result of a) suboptimal levels of addition in food formulation due to regulatory restriction or technical challenges related to solubility and stability of added VD, b) dietary consumption habits, c) lifestyle choices such as minimal sun exposure and potentially the use of sunscreen, or d) variations in bioavailability due to food matrix effect (Cuine et al., 2008; Dahan & Hoffman, 2006; Hossein-nezhad & Holick, 2013; Matsuoka, Wortsman, Hanifan, & Holick, 1988; Norval & Wulf, 2009).

In order to achieve increased loading, stabilization, and bioavailability, as well as to regulate release and enhance absorption, various forms of encapsulated VD have been investigated (Hollander, Muralidhara, & Zimmerman, 1978; Li et al., 2014; Menendez-Aguirre et al., 2014; S. J. Park, Garcia, Shin, & Kim, 2017; Salvia-Trujillo, Fumiaki, Park, & McClements, 2017). Conventionally, oil-solubilized or emulsified forms of VD are added to milk prior to homogenization and thermal treatment, while ready-to-eat cereal products are sprayed with VD during production (Campbell & Marshall, 2016; Johnson, Gordon, & Borenstein, 1988). Complex encapsulation approaches such as entrapments in polymeric nanoparticles (e.g. carboxymethyl chitosan, soy protein isolate, casein micelles) (Menendez-Aguirre et al., 2014; Teng, Luo, & Wang, 2013) are not typically used for food fortification due to : a) complexity of manufacturing processes, b) cost of production, and c) potential effect on food quality and consumer acceptance. In order to address some of these issues, we designed this study to evaluate the feasibility of employing naturally occurring lipid-based colloidal particles, milk fat globule (MFGs), for encapsulation and stabilization of VD₃.

MFGs are naturally occurring lipid droplets consisting of a lipid core surrounded by a tri-layer membrane, the milk fat globule membrane (MFGM), which is comprised primarily of phospholipids and proteins. MFGs core fat is highly complex with approximately 400 different fatty acids that are derived from two sources: a) diet, and b) gut fermentation of dietary fiber and complex carbohydrates (R. G. Jensen, 2002; Parodi, 2004). On the other hand, the MFGM is comprised from polar lipids, sterols, proteins, and trace amounts of other constituents (Singh, 2006; Walstra, Geurts, Walstra, & Wouters, 2005).

MFGs serve as a source of energy-dense lipids, and a reservoir for many naturally occurring lipid-soluble compounds in milk that are introduced to MFGs during its synthesis in the mammary cells. This includes many lipophilic bioactive compounds such as retinol, (3-carotene, and VD at very low concentrations (0.04-2 μg VD₃/L raw milk), as well as some hydrophilic bioactives such as vitamin B2 that was reported to be associated with the MFGM proteins (Kanno, Kanehara, Shirafuji, Tanji, & Imai, 1991; Raynal-Ljutovac, Lagriffoul, Paccard, Guillet, & Chilliard, 2008; Schmid & Walther, 2013; Wijesinha-Bettoni & Burlingame, 2013). The localization of these vital compounds within MFGs stabilizes milk fat against lipid oxidation (S. K. Jensen & Nielsen, 1996). This design feature highlights the structure-function relation of MFGs. Additionally, the digestion of MFGs appear to be highly regulated, due to the presence of the MFGM, in a manner that optimizes the release of lipids and other components, maximizing their bio-accessibility (Bourlieu & Michalski, 2015; Oosting et al., 2014; Rosqvist et al., 2015).

In this study, we aimed to evaluate the encapsulation and gastric stability of VD3 in intact MFGs. The encapsulation process involved the partitioning of VD₃ from the bulk aqueous phase through the MFGM to the lipid core of MFGs. Accordingly, we examined the effects of a) the sources of MFGs (bovine and ovine milks) on encapsulation efficiency of VD₃, and b) the effect of the concentration of food-grade carrier solvent (ethanol) on loading of VD₃ into MFGs from bovine and ovine milks, and C) the stability of VD3 encapsulated in bovine MFGs under in vitro gastric conditions.

In some embodiments, the feasibility of encapsulating and stabilizing VD3 in intact milk fat globules (MFGs) isolated from raw bovine and ovine milks was determined. A passive loading strategy was successfully applied to incorporate VD3 into MFGs. Multi-photon fluorescence microscopy showed that VD3 was distributed uniformly into the lipid core of MFGs. Up to 80 μg/g milk fat and 54 μg/g milk fat, of VD3 were loaded into bovine and ovine MFGs, respectively. These levels are significantly higher than the recommended daily intake of 20 μg of VD3 for adults and children above the age of 4. Encapsulated VD3 in bovine MFGs produced no acid degradation products after two hours of incubation in the simulated gastric fluid at 37° C. In contrast, approximately 8% of un-encapsulated VD3 dispersed in the gastric fluid degraded. These results demonstrated that VD3 could be delivered using this novel encapsulation method, and holds potential for food and pharmaceutical applications.

Role of Milk Fat Globules Origin on Vitamin D3 Encapsulation Yield

Composition of MFGs core and membrane were considered as factors that may influence the encapsulation of VD3 in MFGs. This is based on the understanding that such variations could impact partitioning of VD₃ from the aqueous phase to MFGs via the MFGM.

Compositionally, ovine milk fat is highly saturated in comparison to bovine milk fat, with higher percentage of medium-chain triacylglycerols (C26-C36) and lower proportion of long-chain TAGs (C46-054) (Y. W. Park, Juarez, Ramos, & Haenlein, 2007; Revilla, Escuredo, Gonzalez-Martin, & Palacios, 2017; Vyssotski et al., 2017). Greater degree of saturation ovine milk fat could potentially explain the significantly lower EE % (Tables 7 and 8) that could be caused by decrease in solubility limit of VD₃ in ovine milk fat.

The MFGM is the first barrier to partitioning of VD₃; consequently its composition might play an important role in the process. Prior studies on ovine and bovine MFGMs composition have shown that ovine MFGM has a lower content of short and medium chain fatty acids and greater degree of unsaturation than bovine MFGM (Sanchez-Juanes, Alonso, Zancada, & Hueso, 2009). These variations in MFGM polar lipids composition are expected to modulate the membrane's fluidity. For instance, ovine MFGM is expected to exhibit greater degree of fluidity resulting from disrupted packing of the fatty acid chains due to cis-unsaturated configuration compared to bovine MFGM (Kates, Pugh, & Ferrante, 1984). Increased fluidity could favor partitioning of hydrophobic compounds, however this was not observed in this study. The lack of observed influence of membrane fluidity and composition is potentially due to the presence of ethanol that may induce significant changes in the membrane organization including fluidity, and as a result diminishes the effect of MFGs intrinsic properties on the encapsulation process. In the following sections, we discuss the role of ethanol on the diffusion of VD₃ in MFGs.

In addition to the effect of MFGs source on the partitioning and encapsulation of VD₃, compositional, structural, and morphological variations in MFGs might affect the bioaccessibility and bioavailability of MFGs-encapsulated VD3 (Dahan & Hoffman, 2006; Ozturk, Argin, Ozilgen, & McClements, 2015; Ranade & Cannon, 2011).

### Role of Ethanol on the Encapsulation of Vitamin D3 in Milk Fat Globules

Ethanol was selected as a carrier solvent as it is one of the few organic solvents permitted in some food and drug formulations that can solubilize lipophilic food bioactives such as VD₃, and based on its miscibility in aqueous solutions it enables interaction of these lipophilic bioactives with dispersed MFGs (21CFR172, 2018; 21CFR184.1293, 2018; FDA, 2003). The effect of ethanol was studied as a variable by adjusting its concentration in solution whilst maintaining VD₃ concentration. Our results (Table 7) show that increasing the ethanol concentration to 10% v/v increased the LY and EE % of VD₃ in the MFGs isolated from ovine and bovine milks.

The influence of ethanol on the partitioning of VD₃, a highly hydrophobic compound (Log P 7.13-7.98 (Wishart et al., 2007)), is linked to its well-known effects on permeability of biological membranes and consequent effect on partitioning of molecules across a membrane (Ingram, 1989; Komatsu & Okada, 1997). This effect has been attributed to ethanol forming H-bonds with phospholipid (PL) head groups and competing with the PL-water interactions (Ingram, 1989). This interaction results in an increase in the range of random thermal motion of PL head groups due to weakening of water-PL and inter-lipid interactions, particularly weakening Van der Waals interactions between acyl chains of the PL resulting in fluidization of the outer leaflet (Milburn & Jeffrey, 1989; Slater, Ho, Taddeo, Kelly, & Stubbs, 1993). This leads to the formation of “transient pockets of free volume” (Lieb & Stein, 1969) which increases both partitioning and permeation of VD₃ across the MFGM barrier.

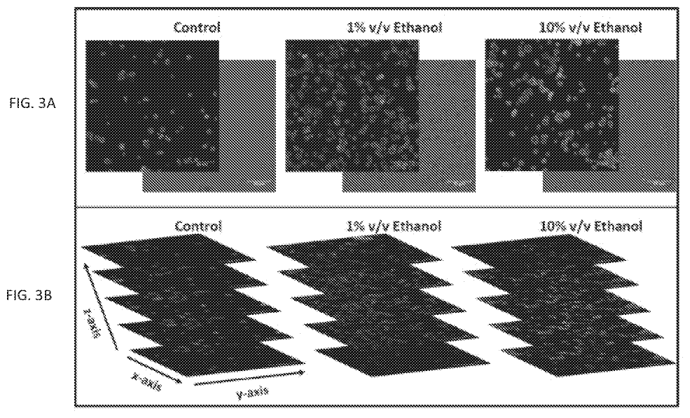

Additionally, ethanol is not expected to diffuse through the membrane as the orientation of the molecule results in placement of polar OH-group near the polar phosphate head group of PL, and the hydrocarbon toward the membrane interior (Löbbecke & Cevc, 1995). Furthermore, Leo et al. (1971) have concluded that that division of ethanol (log P -0.31 (Lien & Gaot, 1995)) between the aqueous phase and the hydrophobic core of cellular bilayer membrane is at a ratio of 1:6, favoring the aqueous phase. Consequently, as the polarity of the MFGM interface increases due to the presence of ethanol at the head-groups of the outer leaflet (Ingram, 1989), a net movement of VD₃ toward MFGM core—a local minima—is favored through few steps of intra-membrane diffusion (Zwolinski, Eyring, & Reese, 1949). Intactness of the MFGM in the presence of ethanol was further validated via CLSM fluorescent imaging (FIGS. 3A and 3B).

### Gastric Stability of Vitamin D3 Encapsulated in Milk Fat Globules

Lipid structures undergo size reduction and emulsification in the gastric phase of digestion (Armand et al., 1996). During such processes, exposure of the lipid-soluble VD to the low pH environment in the stomach might lead to isomerization and consequent loss of its biological activity, which is related to its cis-triene configuration. Under acidic conditions, the acid-catalyzed isomerization product of VD₃, isotachysterol and tachysterol are formed, with the former being the primary isomer (Agarwal, 1990; Jin et al., 2004; Mahmoodani et al., 2017; Seamark, Trafford, & Makin, 1980; Sebrell & Harris, 2014). According to Mahmoodani, isotachysterol is a labile compound that undergoes autoxidation producing IsoOX1, IsoOX2, and IsoOX3 with one, two and three more oxygen atoms than Isotachysterol.

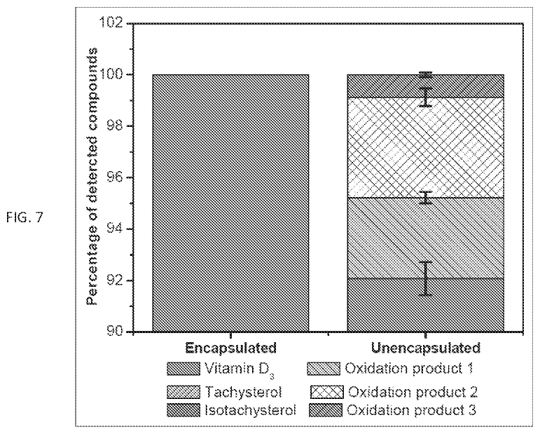

The stability of MFGs-encapsulated VD₃ was investigated under the human physiological conditions including electrolytes composition and concentration, digestive enzyme pepsin, pH 3.0, and 37° C. FIG. 7 presents the LC-MS results after 2 h incubation of MFGs-encapsulated VD₃ and free VD₃ in the simulated gastric fluid. The results clearly show that the MFGs-encapsulated VD₃ did not undergo any observable degradation. The protective effect of MFGs could be attributed to MFGM resistance to disruption under physiological gastric conditions (Vanderghem et al., 2011). Additionally, the minimal lipid digestion expected in the gastric phase means that VD₃, soluble in the triglyceride core of the MFGs, is insulated from direct contact with external hydrogen ions (Garcia, Antona, Robert, Lopez, & Armand, 2014).

In acidic food products such as those containing ascorbic acid as a preservative, the acid-labile compounds, VD₃, have been reported to experience some degradation and loss (Jakobsen & Knuthsen, 2014; Mahmoodani et al., 2017). Encapsulation of VD into MFGs could achieve stabilization of the compound and consequent extended the product shelf life, and allow the addition of VD₃ into a new category of food and drug formulations.

### EXAMPLES

The following examples are non-limiting and other variants within the scope of the art also contemplated.

### Example 12—Materials

Raw bovine milk was sourced from a local farm (Wind River Organics, Palmerston North, New Zealand), and raw sheep milk was a generous donation. Cholecalciferol (HPLC grade), ammonium carbonate, pyrogallol, and pepsin from porcine gastric mucosa (CAS 9001-75-6, P6887) were purchased from Sigma-Aldrich (New South Wales, Australia). Methanol, acetonitrile, formic acid, and isooctane were Optima™ LC/MS grade, chloroform, ethanol, and acetone were HPLC grade, potassium chloride (analytical grade), EDTA Di-sodium salt, magnesium chloride hexahydrate, calcium chloride dehydrate, potassium hydroxide, hydrochloric acid (Hydrochloric Acid, 37%, Certified AR for Analysis, d=1.18, Fisher Chemical), Target2™ PTFE Syringe Filters (0.22 μm pore size), and Millex syringe filter (0.22 μm, polyethersulfone (PSE), 33 mm) were all purchased from Thermo Fisher Scientific (Auckland, New Zealand). Sodium hydrogen chloride, potassium dihydrogen orthophosphate, sodium chloride (Analytical grade), monopotassium phosphate, and sodium bicarbonate were purchased from AnalaR® (BDH Chemicals New Zealand Ltd., Palmerston North, New Zealand). Fluorescence labeling agent DMEQ-TAD (4-[2-(6,7-dimethoxy-4-methyl-3 -oxo-3,4-dihydroquinoxalyl)ethyl]-1,2,4-triazoline-3,5-dione was purchased from Abcam (Cambridge, Mass., U.S.A). Oregon Green™ 488 1,2-Dihexadecanoyl-sn-Glycero-3-Phosphoethanolamine (Oregon Green™ 488 DHPE) was purchased from Thermo Fisher Scientific (Waltham, Mass., U.S.A.). Ultrapure water was used throughout the experimental work.

### Example 13—Isolation of Milk Fat Globules from Raw Milk

Milk was used within 3-4 days of milking during which it was stored at 4° C. Due to limited supply of ovine milk during winter, some aliquots were stored at −80° C. Prior to use, desired volumes of frozen ovine milk were thawed gently over ice at 4° C. for approximately 48 h. Once thawed, the milk was gently mixed at low speed for 1 h at 4° C.

Raw milk was diluted with Milli-Q water at 9:1 water to milk. Cream was separated from diluted milk via centrifugation at 3,000×g for 5 min. Cream was collected and reconstituted in water at 4% w/v concentration. The mixture was then further centrifuged at 3,000×g for 5 min. The cream was then collected and reconstituted to desired weight/volume in water (20% w/v in water).

### Example 14—Particle Size Distribution of Milk Fat Globules

The method of Logan et al. (2014) was used to treat raw milk and MFGs aqueous suspensions from various stages of washing and separation steps. In summary, test materials were treated with 35 mM EDTA (pH 7.0) at 1:1 volume ratio. EDTA, a calcium chelators, was added to milk samples to dissociate casein micelles and eliminate their interference with measurements of particle size distribution of MFGs. Particle size distribution was measured using Malvern Mastersizer 2000 (Malvern Instruments Ltd, Malvern, UK). The refractive index was set to 1.46 for milk fat and 1.33 for water; absorbance was set to 0.001. Measurements were conducted at room temperature. Reported results are averages of two independent biological samples (different lot of milk collected on different dates). Three independent aliquots per milk sample were analyzed.

Data fit was assessed using Mie theory (Mastersizer 2000 software version 6.5). The volume-weighted mean diameter (D_{4,3}) is calculated by the instrument software as follows:

$${D}_{4 , 3} = \frac{\sum {n}_{i} {d}_{i}^{4}}{\sum {n}_{i} {d}_{i}^{3}}$$

where dᵢ is the square root of the upper X lower diameters, and n, is the discrete number of particles.

### Example 15—Effect of Ethanol on the Intactness of the Milk Fat Globule Membrane

To assess the effect of ethanol on the intactness of MFGM, the membrane was probed with a phosphoethanolamine where the head group is labeled with Oregon Green™ 488 (Evers, 2008; Pan & Nitin, 2016). The probe was prepared in chloroform at 1 mg/mL concentration. Five microliter of the label was added to 1 mL of MFGs suspended in water (20% w/v) prepared as described in Example 13, and allowed to incubate—covered—for approximately 10 min at room temperature. At the end of the incubation period, 1 and 10% v/v ethanol were added to the test samples. MFGs were then separated from the water/ethanol solution via centrifugation for 5 min at 3,000×g.

Cream was then reconstituted in water, and a small amount of glycerol was added to reduce the rate of motion of the globules. Experimental procedures were conducted at room temperature. Confocal laser scanning microscopy (CLSM) images were collected on Leica TCS SP8 STED 3× fitted with 40×/1.1 water HC PL APO CS2 objective (Buffalo Grove, Ill., U.S.A.). Excitation and emission were set as 488 nm and 580 nm respectively. Fluorescence emission was collected on Hyd 2 detector at standard mode (499 nm-591 nm), bright field images were collected using PMT Trans detector.

### Example 16—Loading of Vitamin D3 in Milk Fat Globules

Encapsulation of VD3 was achieved by incubation of compound's ethanolic solution with 20% w/v cream suspended in water. Once well mixed by gently inverting the tube, the contents were allowed to incubate in the dark. To assess the influence of carrier solvent concentration on encapsulation of VD₃, two levels of ethanol concentrations, 1 and 10% v/v, were evaluated. For each ethanol concentration, the MFGs were incubated with increasing concentration of VD₃ 50-250 μM at 50 μM interval, for a fixed incubation time of 30 min. To assess the effect of ethanol and VD3 concentrations on the encapsulation efficiency and loading yield of VD₃ in MFGs, a two-way ANOVA was used, followed by post hoc Tukey's analyses to determine statistically significant differences between the experimental groups. Significant values were reported with a 95% confidence interval, i.e. p<0.05.

The effect of incubation time on EE % was assessed by incubating 250 pM of VD₃ at 10% v/v ethanol with bovine MFGs. Incubation time was varied: 15, 30, 45, and 60 min. The effect of incubation time on the encapsulation of VD3 in bovine MFGs was assessed using a t-test with adjusted significance level using Bonferroni correction (p=0.008). These statistical analyses were carried out using Microsoft Excel 2010 (Microsoft Inc., Bellevue, WA, U.S.A.). All procedures were conducted with minimal exposure to light. The experiments were conducted in triplicates.

### Example 17—Extraction of Encapsulated Vitamin D3

At the end of the incubation period with VD₃, cream was separated by centrifuging the suspension at 3,000×g for 5 min Cream was skimmed off the top with particular care not to include any of the aqueous phase. Methanol was added at 50 mg/mL and vortexed vigorously for 1 min. The mixture was then centrifuged at 3,000×g for 30 min. Liquid phase was collected and absorbance was measured at 264 nm. Absorbance blank was prepared fresh during every encapsulation run by introducing ethanol to cream suspended in water. Absorbance of the blank was subtracted from test samples to remove the absorbance of solvent, lipids, and other MFGs components. All experiments were conducted in triplicates.

### Example 18—Determination of Fat Content of Raw Cream

Fat content in cream was measured to calculate the loading yield. The fat was isolated using the same set of preparation steps detailed in Example 13 with the addition of the last centrifugation step to recover cream after at the end of the loading process. The total fat content was determined using a modified Folch method (Folch, Lees, & Sloane-Stanley, 1957) where an additional step of washing the chloroform/water interface with 1:1 water/methanol solution was added to remove milk serum components. The loading yield of VD₃ in milk fat was calculated as follows:

$$LY = \frac{{C}_{E}}{MF}$$

where C_{E} is the VD₃ extracted from MFGs (μg), and MF is the mass of milk fat (g) in MFGs suspension. The extraction procedure was conducted in triplicates using independent milk samples.

### Example 19—Fluorescent Labeling of Vitamin D3 and Multi-Photon Fluorescence Microscopy of the Compound Encapsulated in Milk Fat Globules

Fluorescent imaging was used to confirm the loading of the VD₃ in MFGs and determine the distribution of the compound in the structure. Due to weak auto fluorescence of VD₃, the compound was first conjugated with a fluorescent label, DMEQ-TAD, using a modified version of the method by Shimizu et al. (1997). In summary, DMEQ-TAD stock solution was prepared in dichloromethane at 5 mg/mL concentration. VD₃ and aliquot of DMEQ-TAD stock solution were dissolved in dichloromethane at 1:0.25 molar concentration ratios, respectively. The solution was purged with nitrogen and allowed to mix for 2.5 h in the dark under nitrogen. Equal volume of methanol was added to VD₃-DMEQ-TAD solution to quench the reaction. Finally, all solvents were evaporated under a stream of nitrogen. Remaining solids were stored under nitrogen at −20° C. and used within 24 h.

Labeled VD₃ was re-solubilized in ethanol and added to MFGs suspension as described in Example 15. Excess and free fluorescent label was removed via centrifugal separation of MFGs. Cream layer, collected at the end of the incubation step, was used to acquire microscopic images. For imaging purposes, a small quantity of cream was suspended in water, and a small quantity of glycerol was added to increase the viscosity of the medium and reduce the rate of motion of MFGs particles. CLSM images were collected using Leica TCS SP8 multi-photon microscope (Buffalo Grove, Ill., U.S.A.) equipped with 40×/1.10 HC PL IRAPO water objective. Excitation was achieved using tunable Mai tai deep see laser (690 to 1090 nm) set at resonant mode for high speed scanning, emission was collected on Hyd-RLD 2 detector. Excitation was set at 740 nm and emission was set at 525/50 nm.

### Example 20—Acid-Induced Degradation of Vitamin D3

In order to identify the acid degradation products of VD₃, we performed an acid stress test. HCl aqueous solutions were prepared at pH values: 1, 2, 3, 4, and brought to 37° C., pH value was then corrected for temperature effect. The solutions were briefly purged under a stream of nitrogen. Fresh VD₃ was prepared in methanol at 40 mg/mL then added to acid solutions to reach the final concentration of 0.4 mg/mL. The system was incubated under nitrogen at 37° C. for 2 h. The solutions were neutralized with sodium carbonate to stop the degradation process.

Extraction was performed using a modified method reported by Jin et al. (2004) with ethyl acetate at 1:1 ratio for acid degraded VD₃. The mixture was purged with nitrogen and allowed to incubate in a horizontal shaker for 5 min at room temperature. The mixture was then centrifuged at 2,000×g for 5 min; the top layer was collected and dehydrated over sodium sulfate. Ethyl acetate was evaporated from the top layer under vacuum at 35° C. for 2 h using Savant™ SC50EXP SpeedVac™ equipped with Savant™ refrigerated vapor trap 5105 (Savant Instruments, Inc., Holbrook, N.Y., U.S.A.). The remainder material was reconstituted with 1 mL methanol and filtered into LC vile using 0.22 μm PTFE syringe filter. The procedure was completed in triplicates using independent samples.

### Example 21—Gastric Stability of Vitamin D3 Encapsulated in Milk Fat Globules

Degradation of VD₃ under the simulated gastric conditions was performed on free VD₃ and encapsulated VD₃. The simulated gastric fluid (SGF) was prepared according to the method of Minekus et al. (2014). In summary, electrolyte solution was prepared in Milli-Q water at 1.25× of the final concentration and stored at 37° C. To prepare 10 mL solution to be used in the digestion procedure: 7.5 mL SGF, 6 mg pepsin solubilized in 1.6 mL SGF, 5 μL of 0.3 M CaCl₂(H₂O)₂ prepared in water and filtered through a 0.22 μm PSE syringe filter. The pH value was corrected to reach 3, and if needed, water was added to reach 10mL (approximately 695 pL). The solution was then placed in a 37° C. incubator to equilibrate. Cream separated at the end of the incubation period with VD₃ was suspended in water at 8% w/v and allowed to briefly incubate at 37° C.

The separated cream (250 mg) containing VD₃, obtained as described before, was added to 5 mL of SGF. For the digestion of free VD₃ in methanol, 25 μL of 40 mg/mL solution was added to 4.975 μL of SGF. Solutions were briefly purged with nitrogen and sealed securely. Simulated digestion was carried for 2h at 37° C. At the end of the incubation step, ethanolic pyrogallol solution (10 mL, 1% w/v) was added and vortexed vigorously. Potassium hydroxide (2 mL, 50%w/v) was then added and mixed. The mixture was then incubated at 70° C. for 1 h and vortexed every 15 min, followed by cooling to room temperature. Following this step, ethyl acetate (30 mL) was added and solution was vortexed vigorously for 1 min, followed by addition of water (30 mL). The content was then centrifuged at 3,000×g for 5 min

After centrifugation, the top layer was collected and dehydrated over sodium sulfate. The top layer of ethyl acetate was then collected and removed under vacuum (2 h, 35° C.). Methanol (1 mL) was added to the remaining material, vortexed to mix, then filtered into an LC vial using 0.22 μm PTFE syringe filter. The procedure was completed in triplicates using independent samples.

### Example 22—LC-MS Analysis of Vitamin D3 and its Acid Degradation Products

Analysis was carried using a modified method of Mahmoodani et al. (2017) on a Shimadzu LCMS-8030 Triple Quadrupole Mass Spectrometer (Shimadzu, Kyoto, Japan). The LC separation was performed on an ACE 3 C18-PFP (72×2.1 mm id) column, using 0.1% formic acid in water as mobile phase A and methanol as mobile phase B. Sample (5 58 L) were injected into the chromatographic system running with 75% mobile phase B. The gradient was then changed to 90% mobile phase B within 2 min After 15 min the gradient change to 100% B with 1 min (16 min) and kept at this gradient for more 4 min (20 min). After that the gradient returned to the starting conditions and the column was equilibrated for 5 min before next injection. The column flow rate was 0.3 mL/min and the column temperature was kept at 28° C.

The MS instrument was operated in a positive ion mode. Electrospray ionization was used as LC-MS interface. The desorption line and heat block temperatures were 250° C. and 400° C., respectively. While the drying gas and the nebulizing gas were set at 15 L/min and the 3 L/min, respectively. The MS collision gas pressure was 230 kPa. The instrument operated in the MRM mode using the parameters described in Table 9. Gastric stability measurements were carried using freshly prepared VD₃ encapsulated in bovine MFGs. The procedure was conducted in triplicates using independent samples.

TABLE 9
Compound parameters. IsoOX1: isotachysterol with one oxygen
atom, IsoOX2 two oxygen atom, IsoOX3 three oxygen atom

| | | | Dwell | | | |
|---|---|---|---|---|---|---|
| Instrumental | Precursor | Product | time, | Collision | | |
| parameter | ion, m/z | ion, m/z | msec | Energy, | Q1 (V) | Q3 (V) |
| VD₃ | 385.3 | 259.2 | 100 | −16 | −19 | −19 |
| IsoOX1 | 401.3 | 273.1 | 100 | −16 | −17 | −19 |
| IsoOX2 | 471.3 | 273.0 | 100 | −20 | −17 | −20 |
| IsoOX3 | 433.3 | 272.9 | 100 | −20 | −17 | −19 |

### Example 23—Statistical Analysis

A three-way analysis of variance (ANOVA) was conducted to determine the effect of milk source, ethanol content, concentration of VD₃, and all two-way interactions on the encapsulation efficiency of VD₃ in MFG. In addition, post hoc Tukey's test was applied to determine statistical significance of the observed differences between treatment levels with a confidence interval of 95%. Statistical analyses were performed using RStudio.

### Example 24—Particle Size Distribution of Milk Fat Globules

The washing steps represent the process of isolation of MFGs from raw milk, and the separation of MFGs after loading VD₃. To assess the influence of the washing and separation steps on the physical stability of MFGs, the particle size distribution of MFGs was measured using static light scattering. Additionally, these measurements were used to evaluate the effect of freezing and thawing on ovine MFGs (see 2.2.1). Freezing and thawing of ovine milk did not cause significant difference in the particle size distribution of MFGs (data not shown). Hence, reported particle size distributions of ovine MFGs are the average of fresh ovine milk and quick frozen (-80° C.) then gently thawed milk.

When comparing the volume mean diameter (D_{4,3}) of MFGs in raw milk and after the 3^{rd} washing step, we observed an increase in D_{4,3} from 4.85±0.14 μm to 9.12±0.08 μm, and from 5.35±0.14 μm to 10.07±0.30 μm for bovine and ovine milk respectively (Table 6).

TABLE 6
Particle size distribution of MFGs of bovine and ovine
milks before and after centrifugal separation. Results
are presented as mean ± standard deviation.

MFGs diamter (μm)

| | | | |
|---|---|---|---|
| | Volume mean | 10^{th} percentile | 90^{th} percentile |
| Material | diamter, D_{4, 3} | diamter, d_{0.1} | diamter, d_{0.9} |

Bovine MFGs

| | | | |
|---|---|---|---|
| Raw milk | 4.85 ± 0.14 | 1.84 ± 0.10 | 8.56 ± 0.23 |
| 1^{st} wash | 5.93 ± 0.71 | 2.26 ± 0.17 | 10.66 ± 1.41 |
| 2^{nd} wash | 10.26 ± 1.13 | 2.63 ± 0.07 | 23.00 ± 2.58 |
| 3^{rd} wash | 9.12 ± 0.08 | 2.78 ± 0.08 | 19.04 ± 0.20* |

Ovine MFGs

| | | | |
|---|---|---|---|
| Raw milk | 5.35 ± 0.14 | 2.32 ± 0.00 | 9.24 ± 0.21 |
| 1^{st} wash | 5.52 ± 0.21 | 2.50 ± 0.06 | 9.44 ± 0.50 |
| 2^{nd} wash | 7.87 ± 0.12* | 2.67 ± 0.12 | 15.12 ± 1.46 |
| 3^{rd} wash | 10.07 ± 0.30* | 2.60 ± 0.14 | 21.69 ± 2.14 |

*Denotes statistically significant difference (p < 0.05) in MFGs daimater after separation from raw milk (values are highlighted withing each column)

Using paired t-test, these differences were determined to be statistically significant (p<0.05). This difference is likely attributed to formation of aggregates caused by centrifugal separation and creaming. No free fat was observed, and fluorescent images of the MFGM post separation indicate intactness of the membrane, an essential element in the physical stability of the structure. Similar trend of increasing particle diameter was observed in the 90^{th} percentile diameter (d_{0,9}) with increase in the number of washes, which potentially confirms the formation of aggregates.

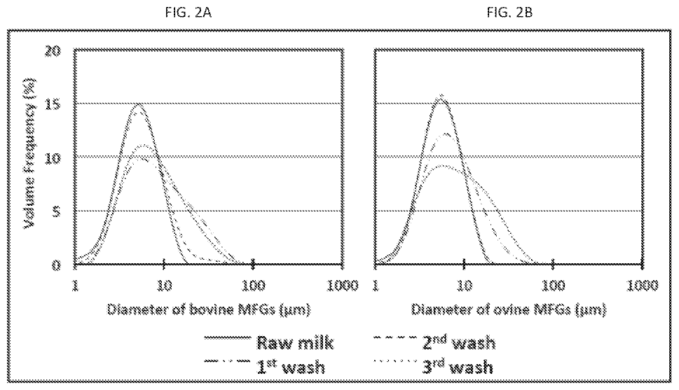

In FIGS. 2A and 2B, the size distribution of MFGs is plotted as volume frequency % versus globules diameter (pm). For both the bovine and ovine MFGs, with the increasing number of washing steps, the MFGs particle size distribution width increased whilst the mean of the particle size distribution remained relatively unchanged. A shoulder appeared in the 10-45 μm range after the third wash of ovine MFGs which can be attributed to formation of some aggregates due to mechanical agitation during the washing steps. In addition, the presence of some aggregates may have been initiated during thawing of the frozen samples.

### Example 25—Effect of Ethanol on Milk Fat Globule Membrane

Fluorescence images of the probed MFGM are shown in FIG. 3A and 3B. Group A show bright field and fluorescence images of control and ethanol treated MFGM. Group B show a z-stack of each treatment. No visible damage to the MFGM was detected upon incubation of MFGs with 1 and 10% v/v ethanol for 30 min. These results indicate that the percentage of ethanol selected for the encapsulation process does not disrupt the structural integrity of MFGs and the MFGM.

### Example 26—Encapsulation of vitamin D3 into milk fat globules

Table 7 shows the encapsulation efficiency (EE %) and loading yield (LY) of VD₃ in MFGs isolated from ovine and bovine sources, at two levels of carrier solvent (1 and 10% v/v).

TABLE 7
Encapsulation efficiency and loading yield of
vitamin D₃ in MFGs of bovine and ovine origins.

| | | | | |
|---|---|---|---|---|
| Compound | | | | |
| concentration | EE %, 1% v/v | LY, 1% v/v | EE %, 10% v/v | LY, 10% v/v |
| (μM) | ethanol | ethanol | ethanol | ethanol |

Bovine MFGs

| | | | | |
|---|---|---|---|---|
| 50 | 20.81 ± 8.82 | 19.78 ± 8.38 | 27.72 ± 7.94 | 26.34 ± 7.55 |
| 100 | 18.45 ± 0.91 | 35.07 ± 1.73 | 21.09 ± 1.10 | 40.09 ± 2.10^{†} |
| 150 | 15.43 ± 1.27 | 44.00 ± 3.63 | 20.67 ± 1.97 | 58.93 ± 5.61^{†¥} |
| 200 | 12.31 ± 3.56 | 46.78 ± 13.52 | 18.51 ± 1.52* | 70.37 ± 5.79^{†‡¥} |
| 250 | 14.28 ± 1.89 | 67.84 ± 9.00 | 16.85 ± 2.94* | 80.07 ± 14.00^{†‡} |

Ovine MFGs

| | | | | |
|---|---|---|---|---|
| 50 | 11.75 ± 0.44 | 11.17 ± 0.41 | 37.83 ± 10.36 | 35.95 ± 9.85 |
| 100 | 10.70 ± 3.12 | 20.35 ± 5.93 | 20.29 ± 4.85* | 38.56 ± 9.23 |
| 150 | 10.17 ± 0.67 | 29.01 ± 1.92 | 18.95 ± 6.34* | 54.03 ± 18.08^{†} |
| 200 | 8.49 ± 1.65 | 32.28 ± 6.26 | 14.30 ± 3.90* | 54.33 ± 14.83^{†} |
| 250 | 8.13 ± 1.27 | 38.62 ± 6.06 | 10.31 ± 1.14* | 49.00 ± 5.43^{†} |

The data presents the effect of ethanol concentration in the aqueous phase (% v/v), and concentration of compound added to MFGs aqueous suspension under fixed incubation time of 30 minutes.
Each value is an average of three independent measurements ± standard deviation.
LY is reported as μg of VD₃ per gram of milk fat.
*Denotes EE % values that significantly differ (p < 0.05) from 50 μM and at 1% v/v ethanol for bovine and ovine MFGs respectively.
^{†}Denotes LY values that significantly differ (p < 0.05) from 50 μM at 1% v/v ethanol.
^{‡}Denotes LY values that significantly differ (p < 0.05) from 100 μM at 1% v/v ethanol.
^{¥}Denotes LY values that significantly differ (p < 0.05) from 250 μM at 1% v/v ethanol.

Overall, increase in compound concentration caused statistically significant increase in LY, but not EE %, with the exception of the cases where 10% v/v ethanol was used with ovine MFGs. In the latter case, a statistically significant decrease in EE % was recorded at VD₃ greater than 50 μM. Overall, the amount of VD₃ loaded into 1 g of milk fat (equivalent to approximately 900 mL of fortified full fat milk which contains 89 IU/100 g per FDA regulations) in the form of MFGs was greater than the RDAs for VD₃ (20 μg).

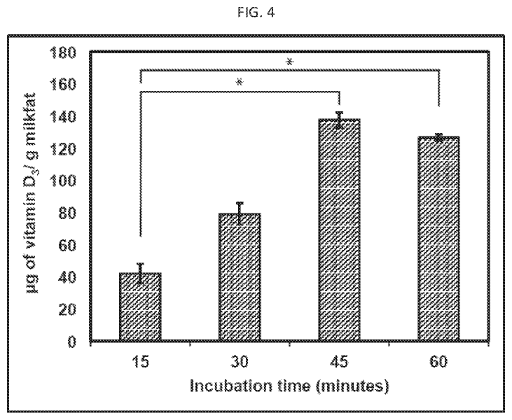

The results in FIG. 4 show a significant increase in encapsulation of VD₃ in bovine MFGs with increasing incubation time up to 45 min using optimal concentration of carrier solvent, i.e. 10% v/v ethanol, and the highest concentration of VD₃ (250 μM) in this study. With extended incubation up to 60 min, the results show a small reduction, approx. 8% in the encapsulation yield of VD₃.

Table 8 summarizes the results of a three-way analysis of variance (ANOVA) used to determine the effect of three variables involved in the encapsulation of VD₃ in MFGs.

TABLE 8
Statistical summary of the effect of encapsulation
process parameters on the encapsulation efficiency (EE %)
of VD₃ in MFGs of bovine and ovine origins

| Parameter | p value |
|---|---|
| MFGs source | 0.00431 * |
| Ethanol concetration | 5.84 × 10⁻⁸ * |
| Vitamin D₃ concetration | 1.50 × 10⁻⁸ * |
| MFGs source and ethanol concetration | 0.01759 * |
| MFGs source and vitamin D₃ cocnetration | 0.11028 |
| Ethanol concetration and vitamin D₃ cocnetration | 0.00133 * |

* Denotes statistically significant values (p < 0.05)

The results show statistically significant effect of MFGs source, ethanol concentration, and VD3 concentration on the EE %. In addition, two-way interactions between these variables are also shown to cause statistically significant effect on EE %. Overall, bovine MFGs, 10% v/v ethanol, and >50μM VD₃ achieved higher EE % than other test conditions.

### Example 27—Localization and Distribution of Vitamin D3 Encapsulated in Milk Fat Globules

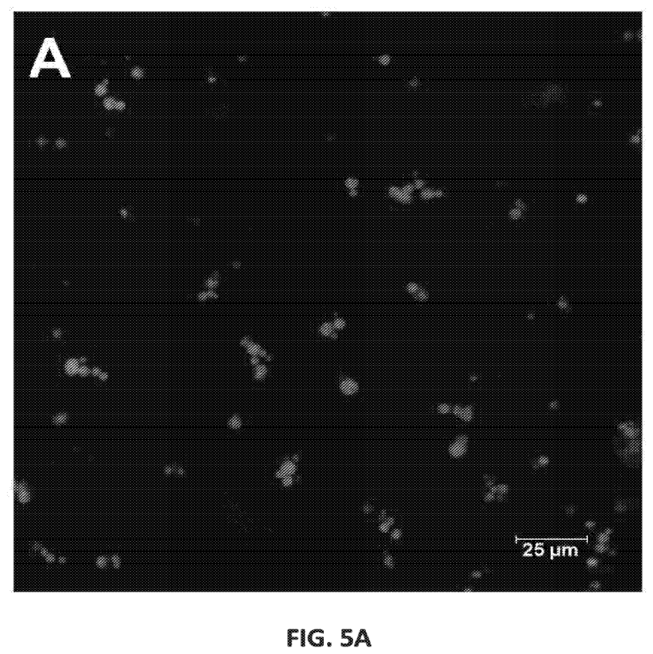

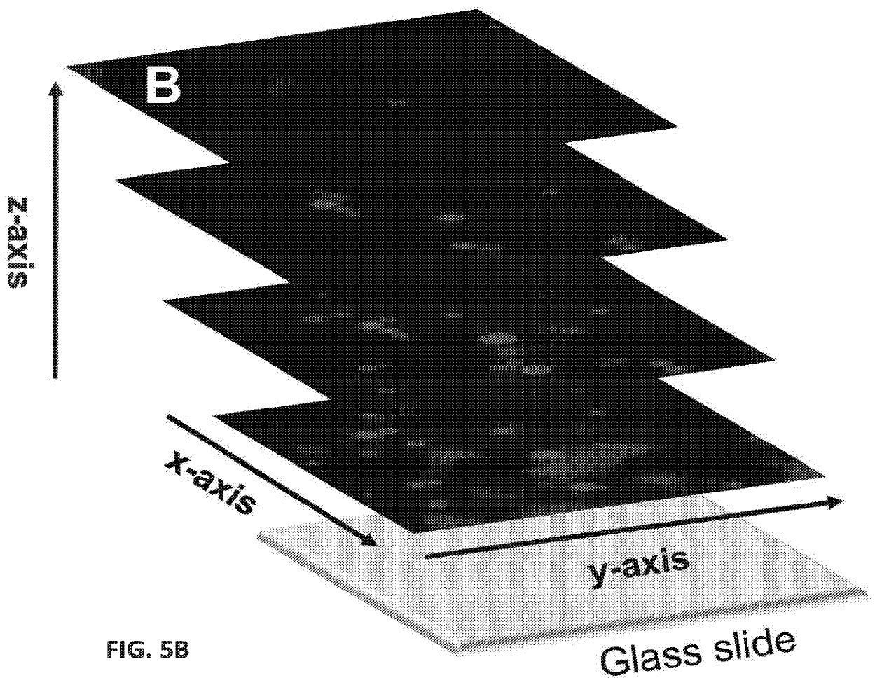

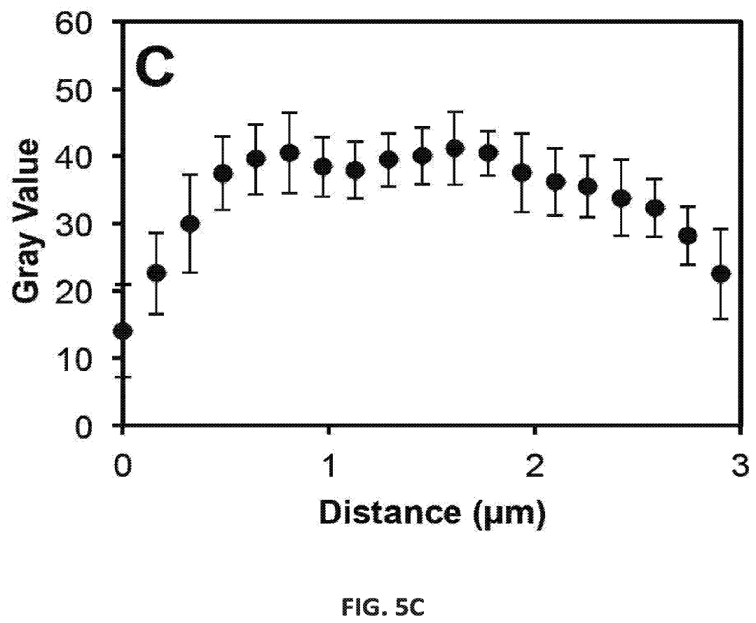

Loading of VD₃ was confirmed visually by multi-photon fluorescence microscopy of bovine MFGs after loading under optimal conditions established based on the results in FIG. 4. Microscopy images (FIGS. 5A-5C) show even distribution of fluorescent signal through the structure of MFGs. These results indicate that the loading of VD3 into MFGs resulted in transmembrane permeation of the compound to the lipid core region of MFGs. The uniformity is also illustrated by the line scan of fluorescence intensity using representative droplets. The average normalized line scan for 6 representative droplets is illustrated in FIG. 5C.

### Example 28—Acidic Degradation of Vitamin D3 Under Gastric-Relevant pH Values

Vitamin D3 is susceptible to degradation under acidic conditions (Mahmoodani et al., 2017; Sebrell & Harris, 2014). To identify and semi-quantify these degradation compounds, degradation of VD₃ was performed based on the methods described before by Jin et al. (2004) and Mahmoodani el al. (2017). Results (not shown) were used to identify VD₃ acid- catalyzed isomers and their autoxidation products (Table 9).

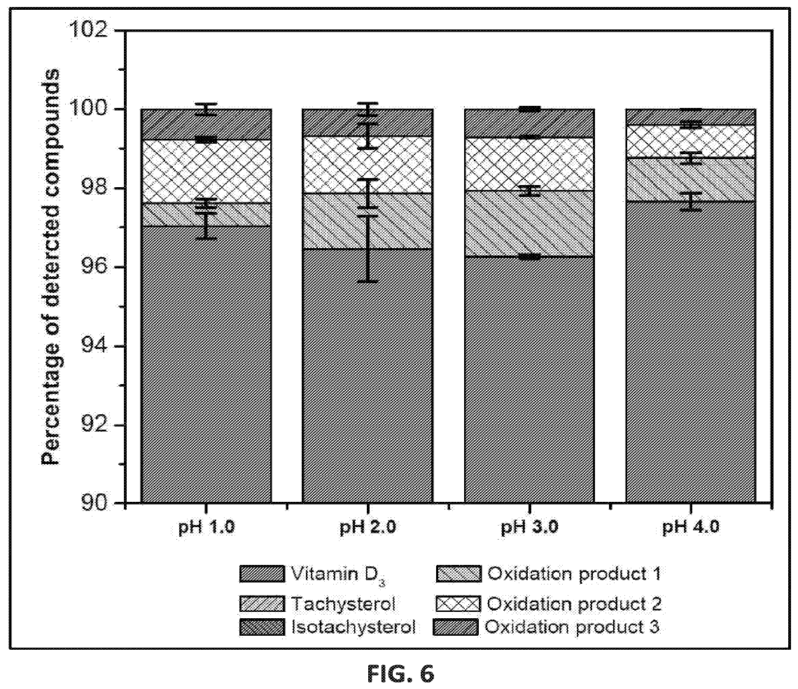

Vitamin D3 was incubated in an acidified aqueous solution in the pH range relevant to the gastric environment, pH 1.0-4.0, in the absence of oxygen and light for 2 h. The results of this treatment are presented in FIG. 6. VD₃ acid-catalysed isomers tachysterol and isotachysterol were not detected; however three of isotachysterol autoxidation products (IsoOX) were detected. Approximately 4% of detected materials were VD₃ degradation products (FIG. 6). IsoOX1 exhibited the greatest degree of fluctuation and pH-dependence, reaching greatest relative concentrations at pH 2.0 and 3.0, and might be further degraded at pH 1, leading to the formation of the other isomer species.

### Example 29—Stability of Milk Fat Globules Encapsulated Vitamin D3 Under the In Vitro Gastric Digestion Conditions

FIG. 7 represents the LC-MS profile of detected compounds at the end of the 2 h gastric digestion. MFGs-encapsulated VD₃ did not generate any detectable VD₃ acid-catalyzed isomers or their autoxidation products. On the other hand, free VD₃ in gastric fluid produced all three of isotachysterol autoxidation products resulting in approximately 6% loss of the free VD₃. Overall, gastric conditions resulted in greater degradation of VD₃ than acid treatments reported in Example 28 in the absence of gastric constituents such as electrolytes and pepsin.

### Example 29.1—Physical Stability of Milk Fat Globules

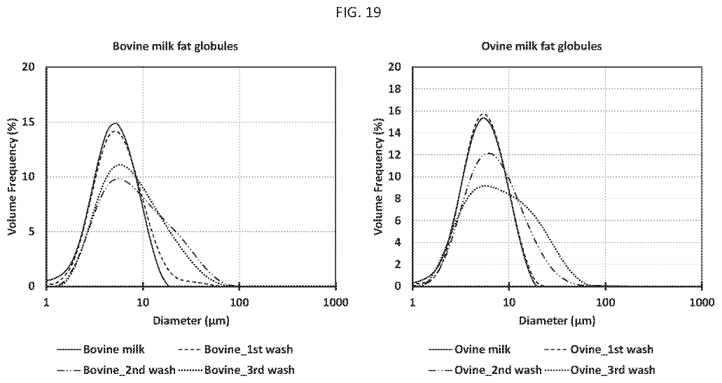

FIG. 19 shows data related to physical stability of milk fat globules. Results show that that milk fat globules retain most of their stability even after several washes.

### Example 29.2—Loading of Milk Fat Globules with VD₃

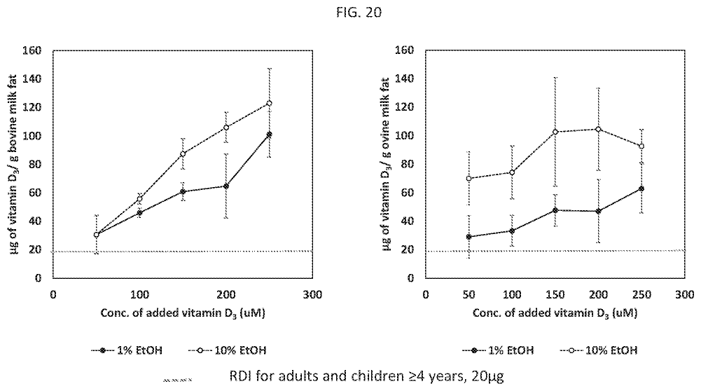

FIG. 20 shows data related to loading of VD₃ in milk fat globules. Results show concentration-dependent loading of VD₃ in milk fat globules.

### Example 29.3—Localization of DMEQ-TAD-Labelled-VD₃ in Milk Fat Globules

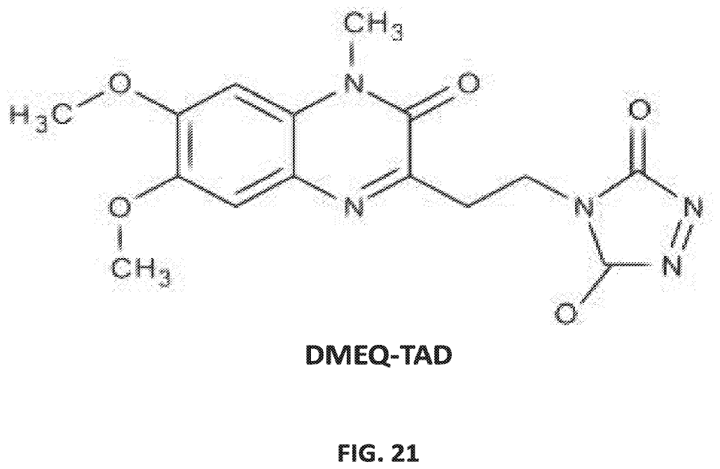

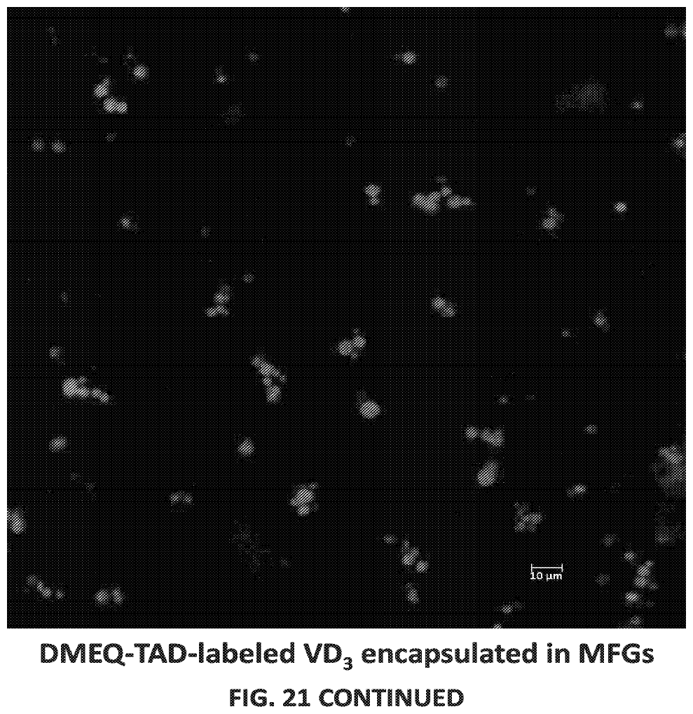

FIG. 21 shows data related to localization of DMEQ-TAD-labelled-VD₃ in milk fat globules. Results show homogenous localization of VD₃ in milk fat globules.

### Summary

The results of this study present a novel method for the encapsulation and stabilization of VD₃ using a naturally available lipid-based colloidal system, MFGs. A passive diffusion process governs the transfer of the lipid-soluble compounds through the MFGM to the triglyceride core. The concentration of the carrier solvent, ethanol, seems to play a great role on the loading process and encapsulation yield. The stability of the bovine MFGs-encapsulated VD₃ under the acidic conditions of the gastric environment was established using LC-MS, which showed that the encapsulated VD₃ was stable within the 2 h incubation. Further work on the release of VD₃ in the gastrointestinal environment is needed to improve our understanding of improved gastric stability of encapsulated VD₃ in MFG. Translation of this promising approach for food fortification applications may require additional investigations on mechanical and physicochemical stability of the ingredients in a complex food matrix. High dosage of VD₃ can be used to encapsulate in MFGs, VD₃ encapsulated in MFGs is protected against acidic degradation in the gastric environment, a do MFGs of varying sources can be used to encapsulate a range of hydrophobic bio-active compounds.

### REFERENCES

All references herein are incorporated by reference in their entireties.

21CFR172. (2018). Subpart C—coatings, films and related substances and subpart D--special dietary and nutritional additives. In D. o. H. a. H. S. U.S. Food and Drug Administration (Ed.), Title 21—Food and drugs. Washington, D.C.

21CFR184.1293. (2018). Subpart B—listing of specific substances sffirmed as GRAS. In D. o. H. a. H. S. U.S. Food and Drug Administration (Ed.), Title 21—Food and drugs. Washington, D.C.

Agarwal, V. K. (1990). A new procedure for the isomerization of vitamin D and its metabolites. Journal of Steroid Biochemistry, 35 (1), 149-150.

Armand, M., Borel, P., Pasquier, B., Dubois, C., Senft, M., Andre, M., Peyrot, J., Salducci, J., & Lairon, D. (1996). Physicochemical characteristics of emulsions during fat digestion in human stomach and duodenum. American Journal of Physiology-Gastrointestinal and Liver Physiology, 271 (1), G172-G183.

Bourlieu, C., & Michalski, M.-C. (2015). Structure—function relationship of the milk fat globule. Current Opinion in Clinical Nutrition & Metabolic Care, 18 (2), 118-127.

Calvo, M. S., & Whiting, S. J. (2013). Survey of current vitamin D food fortification practices in the United States and Canada. The Journal of Steroid Biochemistry and Molecular Biology, 136, 211-213.

Campbell, J. R., & Marshall, R. T. (2016). Dairy Production and Processing: The Science of Milk and Milk Products. Long Grove, Ill.: Waveland Press.

Cuine, J. F., McEvoy, C. L., Charman, W. N., Pouton, C. W., Edwards, G. A., Benameur, H., & Porter, C. J. (2008). Evaluation of the impact of surfactant digestion on the bioavailability of danazol after oral administration of lipidic self-emulsifying formulations to dogs. Journal of Pharmaceutical Sciences, 97 (2), 995-1012.

Dahan, A., & Hoffman, A. (2006). Use of a dynamic in vitro lipolysis model to rationalize oral formulation development for poor water soluble drugs: correlation with in vivo data and the relationship to intra-enterocyte processes in rats. Pharmaceutical Research, 23 (9), 2165-2174.

Darnton-Hill, I., & Nalubola, R. (2002). Fortification strategies to meet micronutrient needs: sucesses and failures. Proceedings of the Nutrition Society, 61 (2), 231-241.

Evers, J. M. (2008). Novel analytical techniques for studying the milk fat globule membrane Massey University Palmerston North, New Zealand.

FDA. (2003). Guidance for industry, Q3C impurities: Residual solvents—tables and list In F. a. D. A. U.S. Department of Health and Human Services (Ed.).

Folch, J., Lees, M., & Sloane-Stanley, G. (1957). A simple method for the isolation and purification of total lipids from animal tissues. Journal of Biological Chemistry, 226 (1), 497-509.

Garcia, C., Antona, C., Robert, B., Lopez, C., & Armand, M. (2014). The size and interfacial composition of milk fat globules are key factors controlling triglycerides bioavailability in simulated human gastro-duodenal digestion. Food Hydrocolloids, 35, 494-504.

Grady, L., & Thakker, K. (1980). Stability of solid drugs: degradation of ergocalciferol (vitamin D2) and cholecalciferol (vitamin D3) at high humidities and elevated temperatures. Journal of Pharmaceutical Sciences, 69 (9), 1099-1102.

Holick, M. F., Binkley, N. C., Bischoff-Ferrari, H. A., Gordon, C. M., Hanley, D. A., Heaney, R. P., Murad, M. H., Weaver, C. M., & Endocrine, S. (2011). Evaluation, treatment, and prevention of vitamin D deficiency: an Endocrine Society clinical practice guideline. The Journal of Clinical Endocrinology and Metabolism, 96 (7), 1911-1930.

Hollander, D., Muralidhara, K., & Zimmerman, A. (1978). Vitamin D-3 intestinal absorption in vivo: influence of fatty acids, bile salts, and perfusate pH on absorption. Gut, 19 (4), 267-272.

Hossein-nezhad, A., & Holick, M. F. (2013). Vitamin D for health: a global perspective. Mayo Clinic Proceedings, 88 (7), 720-755.

Ingram, L. O. (1989). Ethanol tolerance in bacteria. Critical Reviews in Biotechnology, 9 (4), 305-319.

Jakobsen, J., & Knuthsen, P. (2014). Stability of vitamin D in foodstuffs during cooking. Food Chemistry, 148, 170-175.

Jensen, R. G. (2002). The composition of bovine milk lipids: January 1995 to December 2000. Journal of Dairy Science, 85 (2), 295-350.

Jensen, S. K., & Nielsen, K. N. (1996). Tocopherols, retinol, β-carotene and fatty acids in fat globule membrane and fat globule core in cows' milk. Journal of Dairy Research, 63 (04), 565-574.

Jin, X., Yang, X., Yang, L., Liu, Z.-L., & Zhang, F. (2004). Autoxidation of isotachysterol. Tetrahedron, 60 (12), 2881-2888.

Johnson, L., Gordon, H., & Borenstein, B. (1988). Vitamin and mineral fortification of breakfast cereals. Cereal Foods World (USA), 33 (3), 278-283.

Kanno, C., Kanehara, N., Shirafuji, K., Tanji, R., & Imai, T. (1991). Binding form of vitamin B2 in bovine milk: its concentration, distribution and binding linkage. Journal of Nutritional Science and Vitaminology, 37 (1), 15-27.

Kates, M., Pugh, E., & Ferrante, G. (1984). Regulation of membrane fluidity by lipid desaturases. In Membrane Fluidity (pp. 379-395): Springer.

Komatsu, H., & Okada, S. (1997). Effects of ethanol on permeability of phosphatidylcholine/cholesterol mixed liposomal membranes. Chemistry and Physics of Lipids, 85 (1), 67-74.

Leo, A., Hansch, C., & Elkins, D. (1971). Partition coefficients and their uses. Chemical Reviews, 71 (6), 525-616.

Li, W., Peng, H., Ning, F., Yao, L., Luo, M., Zhao, Q., Zhu, X., & Xiong, H. (2014). Amphiphilic chitosan derivative-based core-shell micelles: synthesis, characterisation and properties for sustained release of Vitamin D3. Food Chemistry, 152, 307-315.

Lieb, W., & Stein, W. (1969). Biological membranes behave as non-porous polymeric sheets with respect to the diffusion of non-electrolytes. Nature, 224 (5216), 240.

Lien, E. J., & Gaot, H. (1995). QSAR analysis of skin permeability of various drugs in man as compared to in vivo and in vitro studies in rodents. Pharmaceutical Research, 12 (4), 583-587.

Löbbecke, L., & Cevc, G. (1995). Effects of short-chain alcohols on the phase behavior and interdigitation of phosphatidylcholine bilayer membranes. Biochimica et Biophysica Acta (BBA)-Biomembranes, 1237 (1), 59-69.

Logan, A., Auldist, M., Greenwood, J., & Day, L. (2014). Natural variation of bovine milk fat globule size within a herd. Journal of Dairy Science, 97 (7), 4072-4082.

Mahmoodani, F., Perera, C. O., Fedrizzi, B., Abernethy, G., & Chen, H. (2017). Degradation studies of cholecalciferol (vitamin D3) using HPLC-DAD, UHPLC-MS/MS and chemical derivatization. Food Chemistry, 219, 373-381.

Matsuoka, L. Y., Wortsman, J., Hanifan, N., & Holick, M. F. (1988). Chronic sunscreen use decreases circulating concentrations of 25-hydroxyvitamin D: a preliminary study. Archives of Dermatology, 124 (12), 1802-1804.

Menendez-Aguirre, O., Kessler, A., Stuetz, W., Grune, T., Weiss, J., & Hinrichs, J. (2014). Increased loading of vitamin D2 in reassembled casein micelles with temperature-modulated high pressure treatment. Food Research International, 64, 74-80.

Milburn, M. P., & Jeffrey, K. R. (1989). Dynamics of the phosphate group in phospholipid bilayers. A 31P angular dependent nuclear spin relaxation time study. Biophysical Journal, 56 (3), 543-549.

Minekus, M., Alminger, M., Alvito, P., Ballance, S., Bohn, T., Bourlieu, C., Carriere, F., Boutrou, R., Corredig, M., Dupont, D., Dufour, C., Egger, L., Golding, M., Karakaya, S., Kirkhus, B., Le Feunteun, S., Lesmes, U., Macierzanka, A., Mackie, A., Marze, S., McClements, D. J., Menard, O., Recio, I., Santos, C. N., Singh, R. P., Vegarud, G. E., Wickham, M. S., Weitschies, W., & Brodkorb, A. (2014). A standardised static in vitro digestion method suitable for food—an international consensus. Food & Function, 5 (6), 1113-1124.

Mithal, A., Wahl, D. A., Bonjour, J.-P., Burckhardt, P., Dawson-Hughes, B., Eisman, J. A., Fuleihan, G. E.-H., Josse, R. G., Lips, P., & Morales-Torres, J. (2009). Global vitamin D status and determinants of hypovitaminosis D. Osteoporosis International, 20 (11), 1807-1820.

Norval, M., & Wulf, H. C. (2009). Does chronic sunscreen use reduce vitamin D production to insufficient levels? British Journal of Dermatology, 161 (4), 732-736.

Oosting, A., van Vlies, N., Kegler, D., Schipper, L., Abrahamse-Berkeveld, M., Ringler, S., Verkade, H. J., & van der Beek, E. M. (2014). Effect of dietary lipid structure in early postnatal life on mouse adipose tissue development and function in adulthood. British Journal of Nutrition, 111 (2), 215-226.

Ozturk, B., Argin, S., Ozilgen, M., & McClements, D. J. (2015). Nanoemulsion delivery systems for oil-soluble vitamins: Influence of carrier oil type on lipid digestion and vitamin D3 bioaccessibility. Food Chemistry, 187, 499-506.

Palacios, C., & Gonzalez, L. (2014). Is vitamin D deficiency a major global public health problem? The Journal of Steroid Biochemistry and Molecular Biology, 144, 138-145.

Pan, Y., & Nitin, N. (2016). Real-time measurements to characterize dynamics of emulsion interface during simulated intestinal digestion. Colloids and Surfaces B: Biointerfaces, 141, 233-241.

Park, S. J., Garcia, C. V., Shin, G. H., & Kim, J. T. (2017). Development of nanostructured lipid carriers for the encapsulation and controlled release of vitamin D3. Food Chemistry, 225, 213-219.

Park, Y. W., Juarez, M., Ramos, M., & Haenlein, G. F. W. (2007). Physico-chemical characteristics of goat and sheep milk. Small Ruminant Research, 68 (1-2), 88-113.

Parodi, P. W. (2004). Milk fat in human nutrition. Australian Journal of Dairy Technology, 59 (1), 3-59.

Ranade, V. V., & Cannon, J. B. (2011). Oral Drug Delivery. In V. V. Ranade & J. B. Cannon (Eds.), Drug Delivery Systems (3 ed., pp. 217-231). Boca Raton, Fla.: CRC press.

Raynal-Ljutovac, K., Lagriffoul, G., Paccard, P., Guillet, I., & Chilliard, Y. (2008). Composition of goat and sheep milk products: An update. Small Ruminant Research, 79 (1), 57-72.

Revilla, I., Escuredo, O., Gonzalez-Martin, M. I., & Palacios, C. (2017). Fatty acids and fat-soluble vitamins in ewe's milk predicted by near infrared reflectance spectroscopy. Determination of seasonality. Food Chemistry, 214, 468-477.

Rosqvist, F., Smedman, A., Lindmark-Månsson, H., Paulsson, M., Petrus, P., Straniero, S., Rudling, M., Dahlman, I , & Risérus, U. (2015). Potential role of milk fat globule membrane in modulating plasma lipoproteins, gene expression, and cholesterol metabolism in humans—a randomized study. The American Journal of Clinical Nutrition, 102 (1), 20-30.

Salvia-Trujillo, L., Fumiaki, B., Park, Y., & McClements, D. J. (2017). The influence of lipid droplet size on the oral bioavailability of vitamin D2 encapsulated in emulsions: an in vitro and in vivo study. Food & Function, 8 (2), 767-777.

Sanchez-Juanes, F., Alonso, J., Zancada, L., & Hueso, P. (2009). Distribution and fatty acid content of phospholipids from bovine milk and bovine milk fat globule membranes. International Dairy Journal, 19 (5), 273-278.

Schmid, A., & Walther, B. (2013). Natural vitamin D content in animal products. Advances in Nutrition, 4 (4), 453-462.

Seamark, D., Trafford, D., & Makin, H. (1980). A new procedure for the formation of isotachysterol derivatives of subnanomole quantities of ergocalciferol, cholecalciferol and its metabolites prior to gas liquid chromatography. Journal of Steroid Biochemistry, 13 (9), 1057-1063.

Sebrell, W. H., & Harris, R. S. (2014). The vitamins: chemistry, physiology, pathology, methods: Academic Press.

Shimizu, M., Wang, X., & Yamada, S. (1997). Fluorimetric assay of 1α, 25-dihydroxyvitamin D3 in human plasma.Journal of Chromatography B: Biomedical Sciences and Applications, 690 (1-2), 15-23.

Singh, H. (2006). The milk fat globule membrane—A biophysical system for food applications. Current Opinion in Colloid & Interface Science, 11 (2), 154-163.

Slater, S. J., Ho, C., Taddeo, F. J., Kelly, M. B., & Stubbs, C. D. (1993). Contribution of hydrogen bonding to lipid-lipid interactions in membranes and the role of lipid order: effects of cholesterol, increased phospholipid unsaturation, and ethanol. Biochemistry, 32 (14), 3714-3721.

Spiro, A., & Buttriss, J. (2014). Vitamin D: An overview of vitamin D status and intake in E urope. Nutrition bulletin, 39 (4), 322-350.

Teng, Z., Luo, Y., & Wang, Q. (2013). Carboxymethyl chitosan-soy protein complex nanoparticles for the encapsulation and controlled release of vitamin D(3). Food Chemistry, 141 (1), 524-532.

Tripkovic, L., Lambert, H., Hart, K., Smith, C. P., Bucca, G., Penson, S., Chope, G., Hyppönen, E., Berry, J., & Vieth, R. (2012). Comparison of vitamin D2 and vitamin D3 supplementation in raising serum 25-hydroxyvitamin D status: a systematic review and meta-analysis—. The American Journal of Clinical Nutrition, 95 (6), 1357-1364.

Turner, A. G., Anderson, P. H., & Morris, H. A. (2012). Vitamin D and bone health. Scandinavian Journal of Clinical and Laboratory Investigation, 72 (sup243), 65-72.

Vanderghem, C., Francis, F., Danthine, S., Deroanne, C., Paquot, M., Pauw, E. D., & Blecker, C. (2011). Study on the susceptibility of the bovine milk fat globule membrane proteins to enzymatic hydrolysis and organization of some of the proteins. International Dairy Journal, 21 (5), 312-318.

Vyssotski, M., Lagutin, K., Bloor, S., Mackenzie, A., Scott, D., Broadhurst, M., Day, L., & Samuelsson, L. (2017). Lipid profile of New Zealand sheep milk. Food New Zealand, 17 (1), 32.

Wahl, D., Cooper, C., Ebeling, P., Eggersdorfer, M., Hilger, J., Hoffmann, K., Josse, R., Kanis, J., Mithal, A., & Pierroz, D. (2012). A global representation of vitamin D status in healthy populations. Archives of Osteoporosis, 7 (1-2), 155-172.

Walstra, P., Geurts, T. J., Walstra, P., & Wouters, J. T. (2005). Chapter 2: Milk Components. In Dairy Science and Technology. Boca Raton, Fla., USA: CRC press.

Wijesinha-Bettoni, R., & Burlingame, B. (2013). Milk and dairy product composition. In: Food and Agriculture Organization of the United Nations.

Wishart, D. S., Knox, C., Guo, A. C., Cheng, D., Shrivastava, S., Tzur, D., Gautam, B., & Hassanali, M. (2007). DrugBank: a knowledgebase for drugs, drug actions and drug targets. Nucleic Acids Research, 36 (suppl_1), D901-D906.

Zwolinski, B. J., Eyring, H., & Reese, C. E. (1949). Diffusion and membrane permeability. The Journal of Physical Chemistry, 53 (9), 1426-1453.

Encapsulation and Release of Curcumin from Milk Fat Globules

### Background

Bioactive compounds are defined as components with health promoting qualities that are naturally present in small quantities in food or produced in vivo upon digestion¹. Bioactive compounds are often encapsulated to: a) deliver higher dosage in order to achieve desired biological effect, b) stabilize and protect the compound/s during manufacturing, storage, and delivery, and c) modulate the release and bioaccessibility of the bioactives. Curcumin is one of the most widely researched food-grade bioactives in recent years. Curcumin is isolated from the rhizome of the herb Curcuma longa (turmeric). It is a hydrophobic polyphenolic compound with a log P (octanol/water partitioning coefficient) value of approximately 4.3^{2,3}. Curcumin has been extensively researched for its various physiological activities, including anti-inflammatory, anti-oxidant, anti-cancer, and anti-microbial effects⁴⁻¹⁰. Based on its hydrophobicity and susceptibility to hydrolytic and oxidative damage, curcumin is often incorporated in food and drug products in an emulsified form.

Lipid-based delivery systems such as liposomes, nanoemulsion, and particles stabilized emulsions are commonly used for encapsulation of hydrophobic bioactives. Many of these lipid based encapsulation approaches require the addition of chelators and/or antioxidants to reduce oxidation¹¹⁻¹³, and surfactants and emulsifiers to stabilize these colloidal particles^{14,15}. With increasing demand for more natural products without the addition of exogenous preservatives and stabilizers, there is growing interest in using natural systems such as cell-based carriers for the encapsulation of bioactives, including systems such as yeast and yeast cell wall carriers¹⁶⁻²⁰. Such approaches capitalize on the advantages of natural carriers, such as: the abundance of compositional and structural diversity, relative low cost, and intrinsic physiological compatibility.

This study investigated the utilization of the naturally occurring lipid-based strcutre, milk fat globules (MFGs). MFGs are synthesized via a complex process in the mammary epithelial cells²¹, and are composed of a triglyceride core stabilized in an aqueous medium via multilayer lipoprotein membrane known collectively as milk fat globule membrane (MFGM). MFGs are distinguished from other cellular lipid bodies by their secretion process where cytoplasmic lipid droplets are enveloped by the apical plasma membrane resulting in formation of a unique complex MFGM that is enriched with various bioactive proteins, lipids, and other functional molecules^{22,23}.

MFGs were considered for encapsulation due to their distinct compositional and structural features as well as wide availability, and a relative low cost. Milk fat that resides in the core of the globules is one of the most complex natural lipid with relatively higher concentrations of saturated fats than common emulsions^{24,25}. This could potentially retard the rate of lipids oxidation and consequent degradation of lipid soluble compounds ²⁶. Furthermore, the presence of MFGM have been reported to modulate lipolysis and release of encapsulate compounds²⁷. Compositionally, MFGM possess excellent emulsification properties that are attributed to the unique mixture of many health beneficial phospholipids and proteins^{28,29}.

This study investigated the use of isolated MFGs as carrier for the lipophilic bioactive compound, curcumin. Firstly, partitioning and encapsulation of curcumin in MFGs was confirmed microscopically. Follows, evaluation of the loading kinetics and encapsulation efficiency. Lastly, in vitro gastrointestinal release of encapsulated curcumin was evaluated and correlated with changes in structural properties of MFGs carrier using particle size measurements and fluorescence imaging. In summary, this study provides a novel approach to encapsulate and deliver curcumin using MFGs as a carrier. The results of this study can provide understanding to develop novel value added products using a combination of polyphenolics and MFGs.

In some embodiments, in contrast to conventional encapsulation approaches, a pre-formed and naturally present lipid structures, the milk fat globules (MFGs) for the encapsulation of a model lipid-soluble bioactive, curcumin was evaluated. Intact MFGs were separated from raw milk and dispersed in water, and then curcumin was added in the presence of ethanol. Partitioning of curcumin to the lipid core of MFGs was confirmed using fluorescence imaging. Encapsulation efficiency and loading capacity of curcumin was measured. 1186.65±14.63 μg of curcumin were encapsulated per gram of milk fat, in the presence of 10% v/v of ethanol with an encapsulation efficiency ranging between 59-64%. The study also evaluates the release of curcumin from MFGs carriers under simulated gastrointestinal conditions, and correlates the release with morphological changes in milk fat globules during digestion. The results showed limited release of encapsulated curcumin in the gastric phase (˜27%), and substantial release in the intestinal phase (>80%). Morphological, MFGs showed little changes in the gastric phase, and significant changes in the intestinal phase. Overall, the results of this study demonstrate encapsulation of a lipid-soluble bioactive compound in naturally occurring MFGs using a simple, rapid, and low-energy alternative to conventional encapsulation systems.

Encapsulation of Curcumin into Milk Fat Globules

Encapsulation of curcumin is required for many food and drug applications due to the compound's low aqueous solubility of curcumin (11 ng/mL in buffer solution)³⁶. This study investigated the feasibility of encapsulating this lipid-soluble bioactive compound in intact MFGs, where MFGs isolated from raw milk were suspended in water then combined with an ethanolic solution of curcumin The use of ethanol allows dispersion of curcumin in the MFGs aqueous environment, consequently facilitating the interaction of curcumin with MFGs. In addition, ethanol also enhances the partitioning of curcumin through the MFGM due to its ability to increase the permeability of biological membranes^{37,38}.

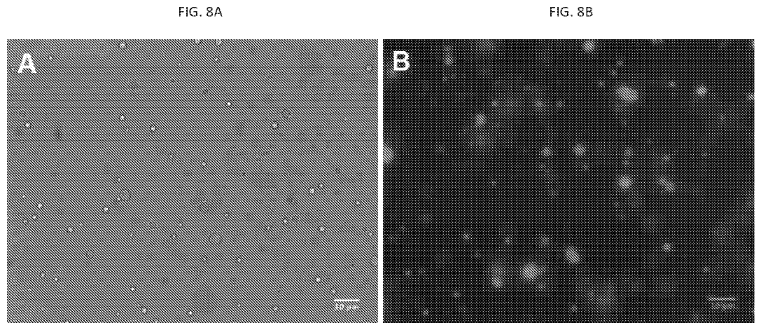

Partitioning of curcumin across the tri-layer membrane of the MFGs and deposition in the lipid core was visualized by CLSM. FIG. 8B shows uniform distribution of the fluorescence signal of the compound in MFGs. Partitioning of curcumin in MFGs can be attributed to curcumin' s ability to bind cellular membranes, solubilize and accumulate in biological bilayers, as well as its association with interfacial protein³⁹⁻⁴². The binding is facilitated by high hydrophobic characteristics of curcumin represented by relatively high octanol/water partitioning coefficient, i.e. log P of 4.3^{2,3}. Additionally, the work of Barry et al. (2009)⁴³ suggests that the presence of curcumin in the bilayer also increases the permeability of biological membranes. This could partly explain the increase in LC of curcumin with an increase in curcumin concentration in the aqueous phase. Once curcumin partitions into the MFGM, concentration gradient and solubility of curcumin in the lipid phase favors its diffusion to the MFGs core.

The LC values reported in this study (Table 10) likely represent the sum total of curcumin solubilized in the TAG core, accumulated in the bilayer, and associated with the MFGM outer leaflet. These values are much higher than those reported in other natural carriers. Young et al. (2017)²⁰ reported an encapsulation yield of curcumin in yeast cells of approximately 400 μg of curcumin/g of yeast. On the other hand, typical LC of curcumin in engineered lipid carriers such as emulsions are between 290 and 300 μg/g for curcumin in canola oil or medium chain TAG⁴⁴⁻⁴⁶, approximately 3 folds lower than LC reported in this study using MFGs carriers.

Release of Curcumin from MFGs Under Simulated Gastrointestinal Digestion Conditions

Lipid digestion is a complex process that involves many physicochemical and enzymatic events. In adult humans, lipolysis occurs in the gastrointestinal track where 6-16% of triglycerides are digested in the gastric phase by gastric lipase, and 42-45% in the intestinal (duodenal) phase by pancreatic lipase⁴⁷. During digestion, oil is emulsified throughout the digestion route from the mouth, through the stomach, into the small intestine. This is a result of the mechanical stress and physiochemical processes of various digestion components that: a) emulsify bulk fat, b) reduce the size of lipid droplets, and c) allow food and digestive fluid components to coat the surface altering the interfacial composition of food emulsions⁴⁸.

Firstly, MFGs were subjected to simulated gastric digestion phase. The gastric phase did not include gastric lipase as limited milk fat digestion is reported in the case of native MFGs coated with MFGM⁴⁹⁻⁵¹. Furthermore, Armand (2008)⁵² have reported limited gastric lipase interaction with MFGM due to the presence of phosphatidylethanolamine and sphingomyelin at the interface. In addition, other studies have also reported gastric limited lipase enzymatic activity (<25%) due to sub-optimal pH values, particularly at fasted state (pH≤3.0)⁵³⁻⁵⁶. The pH conditions used in this study however were near optimal for pepsin activity⁵⁷.

The study of release of curcumin under gastric conditions is important as the release of the poorly water-soluble compound into the acidic aqueous environment of the stomach might result in hydrolysis or precipitation of the compound due to limited solubility in the aqueous phase³⁶. Accordingly, carrier structures that are stable under gastric conditions and are able to provide the desired control on enteric release of bioactives such as curcumin are better suited as for the delivery of lipid-soluble compounds⁵⁸.

The release of curcumin from MFGs in the gastric phase exhibited a burst release of approximately 17% of encapsulated during the first recorded time point at 30 min. After the initial release no significant increase in the release of curcumin was observed with extended incubation up to 2 h. The initial burst release could be a result of release of superficially bound curcumin associated with MFGM interface including the proteinaceous coating of the membrane. Young (2018)⁵⁹ and others^{59,69} reported a limited release of curcumin from yeast cells in gastric fluids (total of 15% in 2 h) exhibiting a similar release profile to that reported in this study. Limited release in the gastric phase was also reported for complex emulsion structures such as Pickering emulsions, Tikekar et al. (2013)³¹ reported 22% release of curcumin at the end of 2 h simulated gastric digestion. On the other hand, release of curcumin from protein-chitosan-based nanocomplexes was reported to be much higher, reaching up to 65% of the encapsulated value⁶¹.

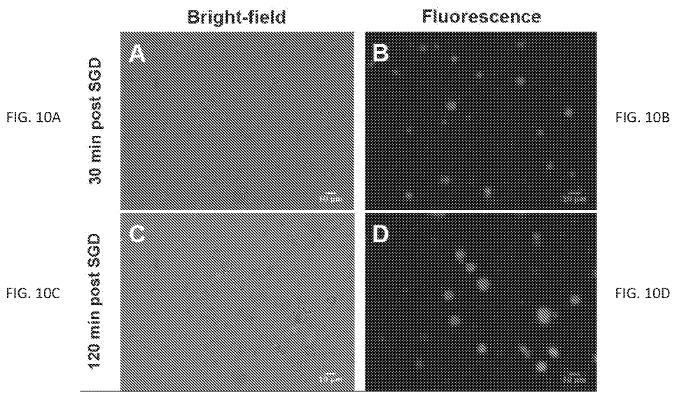

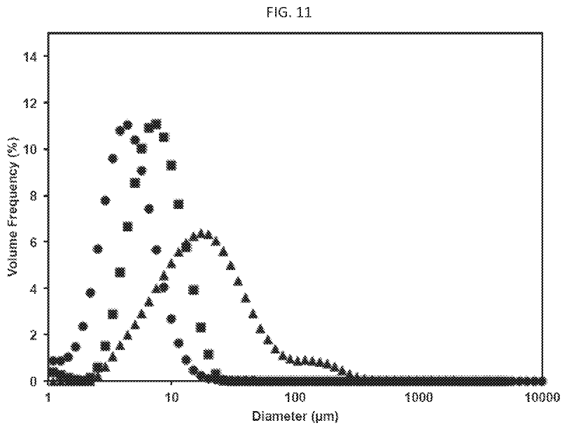

CLSM of MFGs showed intactness of a large number of MFGs after 120 min of SGD (FIGS. 10C and 10D), this can be attributed to MFGM and MFGs lipid core resistance to digestion ⁴⁹. Additionally, distinct lipid formation can be observed surrounding some of the lipid droplets (red arrow in FIG. 10C). These are believed to be amorphous lipids that appear as protrusions and could potentially represent lamellar phase of lipids that form due to alteration in the MFGM phospholipid-protein interactions resulting from conformational changes and/or digestion of some membrane proteins in the gastric phase^{62,63}. The shift in mean particle diameter (FIG. 11) can be attributed to flocculation of MFGs as reported by Ye et al. (2011)⁶⁴, which is associated with modification to MFGM interface such as screening of the electrostatic charge at the interface and digestion of MFGs proteinaceous coating.

Intestinal digestion was carried out without pre-gastric digestion, in the presence of pancreatic lipase and bile salts as the main digestive components. Intestinal lipolysis is essential for the bioaccessibility of lipid-soluble compounds. In the duodenum, triglycerides are digested to monoglycerides and fatty acids that are then micellized with the aid of bile salts and absorbed into the gut. Hence, lipolysis is an essential mechanism for the release and bioaccessibility of encapsulated hydrophobic compounds⁶⁵⁻⁶⁷. Therefore, encapsulation systems with maximized intestinal release are associated with increased bioaccessibility hydrophobic encapsulates.

Release of curcumin from MFGs under SID exhibited significant release within the first 30 min of incubation (>80%); additional release up to 180 min was not statistically significant. Release of curcumin from highly complex carriers such as yeast cells under similar intestinal phase conditions was reported to have a similar profile and total of 80% of encapsulated curcumin released in 3 h⁵⁹. On the other hand, the release kinetics of curcumin from Pickering emulsion were reported to be gradual over the course of 3 h in SIF, totaling 55% of the encapsulated curcumin³¹. The implication of limited gastric release and extensive intestinal release could potentially positively affect the bioaccesibility of curcumin Moreover, the presence of MFGM components such as polar lipids could result in the formation of mixed bile salts, consequently enhancing absorption in the gut²⁷.

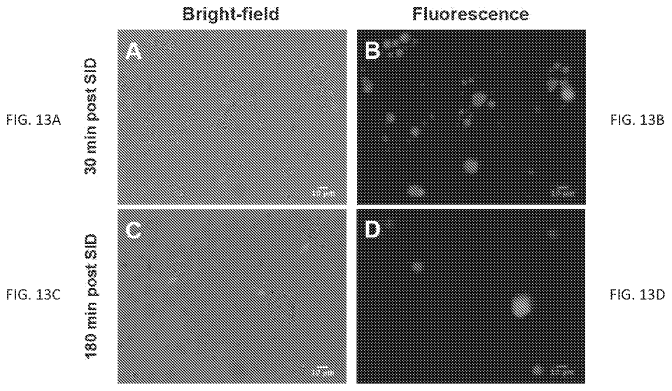

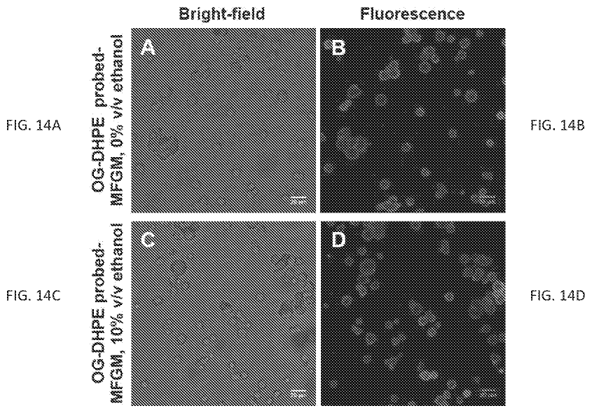

The extent of MFGs morphological changes under SID are evident in the increase of particle size in D_{3,4}, D_{0,1}, D_{0,9} presented in Table 11 as well as broadening and shift of the mean diameter presented in FIG. 11. These changes represent the cumulative result of physical, chemical, and enzymatic effects due to mechanical agitation, elevated temperature, the presence of bio-surfactant (i.e. bile salts) as well as digestive enzymes (i.e. pancreatic lipase). In FIG. 13A blue arrows highlight oil droplets being expelled from crystalline shell, this phenomena was first observed by Patton and Carey (1979)⁶⁸ and more recently by Gallier et al. (2012 & 2013)^{49,69}. Needle-shaped crystals in FIG. 13C are likely composed of free fatty acids resulting from milk fat digestion⁶⁹.

Endogenous fluorescence of crucmin in the lipid phase at the end of gastric and intestinal digestion (FIG. 10D and FIG. 13D) could indicate stability of the compound against hydrolysis. Curcumin is only slightly soluble in water at alkaline pH, and undergoes hydrolysis when in contact with water⁷⁰. This results in quenching of the fluorescence intensity⁷¹.

### EXAMPLES

The following examples are non-limiting and other variants within the scope of the art also contemplated.

### Example 30—Materials

Raw whole bovine milk was procured from local markets (Organic Pastures, Fresno, Calif., U.S.A.). Nanopure distilled water (16 MΩ-cm) obtained using Millipore® Milli-Q RG water purification system (Billerica, Mass., U.S.A.). Ethanol (200 proof, KOPTEC), curcumin (Curcumin from curcuma longa (Turmeric)), tetrahydrofuran (THF), calcium chloride, potassium phosphate monobasic, bile salts, pepsin from porcine gastric mucosa, and lipase type II from porcine pancreas were obtained from Sigma-Aldrich (St. Louis, Mo., U.S.A.). Slide-A-Lyzer™ G2 dialysis cassettes (3.5K MWCO), chloroform, hydrochloric acid, and sodium chloride were purchased from Thermo Fisher Scientific Inc. (Pittsburgh, Pa., U.S.A.).

### Example 31—Isolation of Milk Fat Globules

Raw milk was used within 3-4 days of purchasing during which it was stored at 4° C. MFGs were isolated using a modified form of the method by Gallier et al. (2010) ³° . In summary, raw milk was diluted using water (9:1 water to milk ratio). The diluted solution was centrifuged at 3000×g for 5 minutes; the cream layer was collected and dispersed in water to create 10% w/v suspension.

### Example 32—Quantification of Milk Fat in Cream

Raw cream that was separated following the method stated in Example 31 was used for the quantification of oil content per gram of raw cream. A 10% w/v cream was dispersed in water and homogenized using a hand-held disperser (ULTRA TURRAX, IKA works, Wilmington, N.C., USA) at 11,000 rpm for 2 min. Fat was then extracted using chloroform at 5:1 homogenized cream to chloroform ratio, followed by centrifugation at 10,000×g for 30 min at room temperature. The two immiscible phases were separated. Acidity of the aqueous phase adjusted to pH 4.5 using 1M HCl to precipitate remaining MFGM proteins and associated serum proteins. A final extraction of oil from the acidified aqueous phase was carried by adding chloroform to the acidified aqueous phase in the ratio of 1:25, followed by centrifugation at 10,000×g for 10 min at room temperature. Chloroform extracts were combined and the solvent was removed under vacuum at room temperature for 24 h. Mass of the isolated lipid from the sample was measured gravimetrically. Measurements were completed in triplicated from independent raw milk samples.

### Example 33—Loading of Curcumin in Milk Fat Globules

Curcumin solution in ethanol was prepared at 2 mg/mL concentration. Aliquots of curcumin ethanolic solution were added to 10% w/v MFGs in water suspension and incubated for 10 min. Concentrations of added curcumin, as well as the v/v % of added ethanol were adjusted as shown in Table 9.

TABLE 9
The effect of incubation time on the EE % of curcumin in MFGs.

| Incubation time (min) | EE % |
|---|---|
| 10 | 62.36 ± 1.85 |
| 20 | 66.43 ± 4.32 |
| 30 | 61.20 ± 1.78 |
| 40 | 59.75 ± 2.10 |
| 50 | 57.24 ± 0.83* |
| 60 | 64.30 ± 2.42 |

*Denotes values which significantly different (p < 0.05).

Incubation was completed in the dark to prevent photodegradation of curcumin. To study the kinetics of curcumin encapsulation, a fixed concentration of curcumin and ethanol were added to the aqueous suspension of raw cream, and incubation time was adjusted from 10-60 min at 10 min intervals. Encapsulations measurements were conducted in triplicates.

### Example 34—Extraction and Quantification of Curcumin Encapsulated in Milk Fat Globules

At the end of the incubation period, encapsulated curcumin was extracted from MFGs using THF at 1:10 volume ratio of cream suspension to THF. The mixture was then vortexed rigorously, followed by centrifugation at 16,000×g for 10 min. Curcumin concentration was then measured using a UV-vis spectrophotometer (GENESYS 10S Series, Thermo Scientific, Pittsburgh, Pa., USA) where λₘₐₓ of curcumin in THF was detected at 422 nm. Blanks samples of MFGs treated with ethanol were used to subtract background absorbance caused by MFGs components. A standard curve of curcumin in THF was used to convert relative absorbance units to concentration units. Curcumin containing samples were kept covered throughout the extraction and measurements steps. The encapsulation efficiency was calculated as follows:

$$\% EE = \frac{{C}_{E}}{{C}_{T}} \times 100$$

where C_{E} is the mass of curcumin extracted from MFGs, and C_{E} is the mass of curcumin added initially. The loading capacity was calculated as follows:

$$LC = \frac{{C}_{E}}{MF}$$

where C_{E} is the mass of curcumin extracted from MFGs, and MF is the mass of milk fat in MFGs suspension.

### Example 35—Fluorescence Imaging of Curcumin Encapsulated in Milk Fat Globules

A representative MFGs sample loaded with 600 μg curcumin/g raw cream as described in Example 33 was examined under a microscope using a 40× oil immersion objective (Olympus UPlanFL, 40×/1.30 oil, Olympus Inc., Center Valley, Pa., USA). Fluorescence images were obtained using an inverted optical microscopy, Olympus IX-7 (Olympus Inc., Center Valley, Pa., USA). The images were acquired using an excitation filter: 480/30 nm, and emission filter: 570/60 nm. The camera exposure time for image acquisition was set to 100 ms.

### Example 36—Release of Curcumin from Milk Fat Globules Under Simulated Gastric Digestion Conditions

MFGs samples loaded with curcumin at 600 μg curcumin/g raw cream were used for the simulated gastric digestion measurements. Simulated gastric digestion was carried in a dialysis cassette with a molecular weight cut off of 3500 Da. MFGs suspension was combined with the simulated gastric fluids (SGF) in the dialysis cassette suspended in a 1L of enzyme-free SGF³¹. SGF was prepared according to the method reported by Sarkar et al. (2009)³² where 5 g/L sodium chloride solution was acidified to pH 1.2 starting using 12.1 M HCl. Fine adjustments of the solution's pH were accomplished using 1M of HCl or NaOH. SGF was then stored at 37° C. When needed the pH was further corrected to account for temperature effects.

Nine milliliter of MFGs suspension (at 37° C.), and 1 mL of SGF (at 37° C.) containing additional 0.018 g NaCl and 0.032 g pepsin, were combined in a dialysis cassette. The cassette containing MFGs sample was then allowed to float and rotate in a beaker containing 1L of enzyme-free SGF, with the aid of a magnetic stirrer and a stir plate set at approximately 450 rpm. This arrangement allows the released compound and degraded MFGs structures to diffuse out of the cassette as a result of concentration gradient. Equilibration of curcumin concentration in the two systems (inside and outside of the dialysis cassette) is not expected to occur over the course of digestion time (2h) due to a large excess (100 fold) of the outer chamber volume and limited water solubility of curcumin The simulated gastric system was maintained at 37±2° C. for the 2h duration of digestion. A hundred microliter aliquots were collected from the cassette every 30 min; the concentration of curcumin was measured using the previously described method in Example 34. Reported results are the average measurements of three independent runs. Additional 20 μL aliquot was collected and examined microscopically in order to study morphological changes of MFGs structure. Fluorescence imaging of curcumin was completed using the same procedure detailed in Example 35.

### Example 37—Release of Curcumin from Milk Fat Globules Under Simulated Intestinal Digestion Conditions

Simulated intestinal digestion was conducted using as a modified procedure based on an earlier reported study by Tikekar and Pan (2013)³¹. In summary, simulated intestinal fluid (SIF) comprised of 0.05 M potassium dihydrogen phosphate solution, 6.66 mg/mL calcium chloride, and 50 mg/mL bile salts. The addition of calcium chloride in the SIF causes some aggregation of bile salts. These aggregates were removed via centrifugation. The supernatant was removed and pH of the supernatant was adjusted to 7.5. The solution was equilibrated to 37° C., and 20 mg/mL of lipase were added directly prior to initiating the digestion protocol.

The SIF was mixed with the MFGs suspension (held at 37° C.) at 1:1 volumetric ratio and incubated in a shaking incubator at 37° C. and 95 rpm for a total of 3 h. Separate tubes were designated for each time point. At each time point (data were collected every 30 min for a total of 3 h), approximately 20 μL aliquot were collected for imaging purposes to study morphological changes of MFGs structure following the method of Example 35. The SIF/MFGs mixture was centrifuged at 25° C. and 47,808×g for 30 min using Sorvall RC6⁺ Centrifuge, SS-34 fixed angel rotor (Thermo Scientific, Waltham, Mass., USA). Release was defined as the measured concentration of curcumin in the transparent micellar phase. The concentration of curcumin in the micellar phase was measured based on UV-vis absorption at 425 nm. The micellar phase sample from digested control MFGs suspension served as a blank. Reported results are the average measurements of three independent runs.

### Example 38—Particle Size Measurements of Milk Fat Globules During Simulated Gastrointestinal Digestion Conditions

Changes in MFGs particle size distribution during the digestion process was observed using Mastersizer 2000 particle analyzer (Malvern Instruments, Worcestershire, UK). The instrument was set to the following variables: particle refractive index=1.45, particle absorption index=0.0, dispersant (Milli-Q water) refractive index=1.33, model: general purpose spherical. Measurements were made using independent triplicate samples per time point. Furthermore, the milk fat globules were isolated from two different batches of milk. Thus, each time point reading had 6 independent measurements.

### Example 39—Statistical Analysis

Statistical analysis was completed using Microsoft Excel 2010 (Microsoft Inc., Bellevue, Wash., USA). Statistical significance was carried out using one-way ANOVA followed by post hoc Tukey's analysis to determine significant difference between groups. Significance values were reported with a 95% confidence interval, i.e. p<0.05. Experimental data were collected in triplicates unless stated otherwise.

### Example 40—Encapsulation of Curcumin in Intact Milk Fat Globules

Encapsulation of curcumin in MFGs was achieved by co-incubation of MFGs with a water-ethanol mixture containing curcumin. The partitioning of curcumin in MFGs was first validated microscopically. FIGS. 8A and 8B show bright field and fluorescence images of MFGs with curcumin, respectively. FIG. 8A illustrates intact MFGs after incubation with water-ethanol mixture containing curcumin. The imaging results indicate MFGs maintain shape and integrity of the globules upon incubation with the aqueous solution containing ethanol and curcumin Further validation of membrane integrity is illustrated in FIGS. 14A-14D. FIG. 8B shows uniform distribution of curcumin endogenous fluorescence, corresponding to localization of the compound in the lipid core region of MFGs. These results demonstrate permeation of curcumin through the MFGM to the lipid core of MFGs.

To identify the influence of incubation time on the encapsulation efficiency and loading capacity, the incubation time was varied between 10-60 min as described in Example 33. Encapsulation efficiencies (EE %) as a function of incubation time for a fixed concentration of curcumin are shown in Table 9. EE % of curcumin in MFGs ranged between 57% and 66%. Using one-way ANOVA followed by Tukey's post hoc test, a small, yet statistically significant difference in EE % of curcumin in MFGs was observed after 50 min of incubation, compared to other time points tested in this study. In fact, this value was also the lowest calculated EE %. Due to limited influence of extended incubation time on EE % of curcumin in MFGs, incubation time of 10 min was selected for the following experiments.

The results in Table 10 present the EE % and LC of curcumin in MFGs as a function of increasing curcumin concentration (200-2000 μg/g of raw cream) in the aqueous phase, for a fixed incubation time of 10 min.

TABLE 10
The effects of curcumin and ethanol concentrations on the
EE % and LC % into MFGs and reported per g of milk fat.

| Concentration of | | | |
|---|---|---|---|
| added curcumin/ | v/v % of | | LC %, curcumin/ |
| cream (μg/g) | ethanol | EE % | milk fat (μg/g) |
| 200 | 1 | 62.09 ± 1.46 | 368.11 ± 2.92 ^{a} |
| 400 | 2 | 61.17 ± 2.13 | 244.69 ± 8.53 ^{b} |
| 600 | 3 | 62.10 ± 2.54 | 372.60 ± 15.27 ^{c} |
| 1000 | 5 | 66.56 ± 0.13* | 665.55 ± 1.31 ^{d} |
| 1600 | 8 | 62.55 ± 0.37 | 1000.76 ± 5.97 ^{e} |
| 2000 | 10 | 59.33 ± 0.73 | 1186.65 ± 14.63 |

Fat content was measured to be 4.71 ± 0.29 g/10 g of cream.
EE, encapsulation efficiency;
LC. loading capacity.
Different letters and * denotes values which significantly different (p < 0.05).

As a result of increasing curcumin concentration, the ethanol concentration in the system increased from 1 to 10% v/v. The results show significant increase (p<0.05) in LC with an increase in the concentration of curcumin in the aqueous phase. The LC value increased from 368 μg/g milk fat to 1186 μg/g milk fat, representing a 3 fold increase, with an increase in curcumin concentration while the EE % did not change significantly except at 5% v/v ethanol solution. The EE % ranged from 59% to 66% for all the conditions tested for these set of experiments.

### Example 41—Effect of Simulated Gastric Digestion on Milk Fat Globules Carriers

To study the release of encapsulated curcumin, the delivery system was evaluated using in vitro simulated digestion conditions with appropriate electrolytes and enzymatic composition, temperature and pH conditions³³⁻³⁵. Physical changes of MFGs were measured using fluorescence microscopy and particle size analysis. Release of encapsulated compound during gastric and intestinal phases was studied independently to allow for comprehensive understanding of the digestive events.

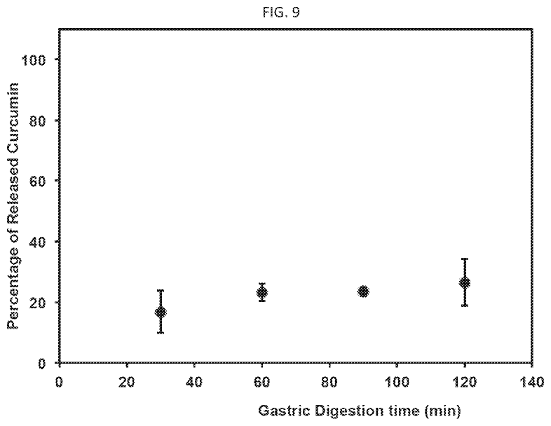

FIG. 9 shows the release of curcumin from MFGs under simulated gastric digestion. A small fraction, approximately 27%, of total curcumin encapsulation in MFGs was released after 2 h of incubation under simulated gastric conditions. Most of this release was observed during the first 30 min of incubation under gastric conditions. Additional release of curcumin (from 30-150 min) was not statistically significant (p>0.05).

Microscopic images of MFGs under simulated gastric digestion are shown in FIGS. 10A-10D. After 30 min of gastric digestion, MFGs appeared intact as shown in FIG. 10A. However at the end of the digestion period (2 h), irregular boundaries of lipid droplets were observed. These boundaries are highlighted with the red arrows in FIG. 10C. Throughout the digestion time, fluorescent images of encapsulated curcumin show localization in the lipid pool of the MFGs (FIGS. 10B and 10D). Table 11 shows changes in particle size during gastric digestion. Statistically significant (p<0.05) increase in MFGs size is recorded compared to initial particle size prior to gastric digestion.

TABLE 11
MFGs diameter as a function of digestion time.

| | | | |
|---|---|---|---|
| | Volume mean | 10^{th} percentile | 90^{th} percentile |
| Time (min) | diameter, D_{4, 3} | diameter, D_{0.1} | diameter, D_{0.9} |

MFGs particle size distribution (μm)

| | | | |
|---|---|---|---|
| Prior to | 4.58 ± 0.40 | 2.02 ± 0.14 | 7.75 ± 0.87 |
| digestion | | | |

Simulated gastric digestion

| | | | |
|---|---|---|---|
| 30 | 5.92 ± 0.16 ^{a} | 2.75 ± 0.12 ^{a} | 10.01 ± 0.38 ^{a} |
| 60 | 6.58 ± 0.16 ^{b} | 2.89 ± 0.21 ^{b} | 11.10 ± 0.23 ^{b} |
| 90 | 6.83 ± 0.15 ^{c} | 3.34 ± 0.29 ^{c} | 11.34 ± 0.13 ^{c} |
| 120 | 7.42 ± 0.20 ^{d} | 3.63 ± 0.32 ^{d} | 12.42 ± 0.29 ^{d} |

Simulated intestinal digestion

| | | | |
|---|---|---|---|
| 30 | 27.85 ± 5.15 ^{a} | 6.22 ± 1.63 ^{a} | 114.87 ± 52.42 ^{a} |
| 60 | 24.53 ± 4.86 ^{b} | 6.15 ± 1.57 ^{b} | 67.24 ± 28.37 ^{b} |
| 90 | 35.85 ± 16.92 ^{c} | 6.52 ± 1.23 ^{c} | 94.55 ± 34.40 ^{c} |
| 120 | 27.52 ± 6.86 ^{d} | 6.41 ± 1.09 ^{d} | 98.10 ± 36.34 ^{d} |
| 150 | 26.56 ± 4.14 ^{e} | 6.24 ± 1.15 ^{e} | 72.66 ± 23.05 ^{e} |
| 180 | 32.27 ± 13.17 ^{f} | 5.86 ± 1.45 ^{f} | 80.17 ± 40.13 ^{f} |

Different letters indicate statistically significant difference within each column in comparison the particle size prior to digestion (p < 0.05).

FIG. 11 shows shift in the distribution of MFGs diameter at the end of the digestion period (2h) to larger values without noticeable difference in the overall shape of the distribution curve.

### Example 42—Effect of Simulated Intestinal Digestion on Milk Fat Globules Carriers

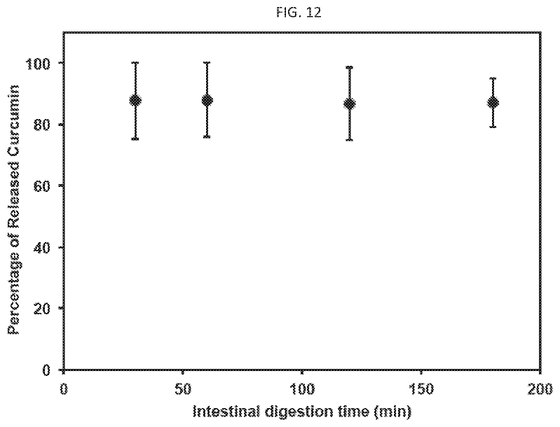

Release of curcumin from MFGs as a result of simulated intestinal digestion is illustrated in FIG. 12. The first recorded time point of 30 min registered approximately 88% release of the encapsulated curcumin Following this initial time point, insignificant change in the release of curcumin was recorded during the following 150 min of digestion time. Within the first 30 min of intestinal digestion, various morphological changes can be observed including some aggregation of MFGs. Bright-field images at 30 min (FIG. 13A) shows expulsion of oil droplets indicated by blue arrows. FIG. 13B shows fluorescent images. FIG. 13C shows needle-shaped crystals at the peripheral of damaged MFGs structures (red arrows). At the end of the digestion step at 180 min, fewer intact MFGs were observed. Fluorescent images (FIG. 13D) show localization of curcumin in the digested MFGs structure.

Particle size distribution of MFGs post intestinal digestion is shown in FIG. 11. A peak shift was observed in particle size distribution curve, from approximately 4.4 μm to 17.4 μm, as well as a widening of the particle size distribution. Additionally, a minor secondary peak appeared toward larger diameter particles (>90 μm) indicating formation of large aggregates of MFGs. Increase in D_{3,4}, D_{0.1}, and D_{0.9} were all statistically significant after intestinal digestion compared to particle size measurements prior to digestion.

### Example 43

To validate the integrity of milk fat globule member in the presence of 10% v/v ethanol in the continuous aqueous phase, the MFGM was probed with a fluorescently labeled phosphoethanolamine (Oregon Green™ 488 DHPE was purchased from Thermo Fisher Scientific Waltham, Mass., U.S.A.). To summarize, the label was prepared in chloroform (1 mg/mL), then 2.5 pi of the label was added to 1 mL of MFGs suspended in water and allowed to incubate for 10 min in the dark. Ethanol was then added at 10% v/v and incubated for no less than 10 minutes. For imaging purposes, a small amount of glycerol was added to the mixture reduce the rate of motion of the globules. Confocal laser scanning microscopy (CLSM) images were collected on Leica TCS SP8 STED 3× fitted with 40x/1.1 water HC PL APO CS2 objective (Buffalo Grove, IL, U.S.A.). Excitation and emission were set as 488 nm and 580 nm respectively. Fluorescence emission was collected on Hyd 2 detector at standard mode (499 nm-591 nm), bright field images were collected using PMT Trans detector.

### Summary

The results of this study confirm the feasibility of encapsulating hydrophobic bioactive compounds into the preformed complex lipid structures, MFGs. Greater than 1000 μg of curcumin were encapsulated per gram of milk fat in the form of MFGs via simple diffusion driven by hydrophobic affinity of curcumin. This simple method enables at least 3 folds increase in encapsulation yield of curcumin compared to the reported loading values in emulsions formed using edible oils. In vitro release of curcumin from MFGs carriers exhibited minimal release in gastric phase (approximately 27% in 2h), and extensive release under intestinal conditions (>80%). These results suggest that MFGs carrier could be used successfully for encapsulation and oral delivery of lipid-soluble compounds.

### REFERENCES

All references herein are incorporated by reference in their entireties.

1. Kitts D D. Bioactive substances in food: Identification and potential uses. Can J Physiol Pharmacol. 1994; 72(4):423-34.

2. Yamagami C, Araki K, Ohnishi K, Hanasato K, Inaba H, Aono M, et al. Measurement and prediction of hydrophobicity parameters for highly lipophilic compounds: Application of the hplc column-switching technique to measurement of log p of diarylpyrazines. J Pharm Sci. 1999; 88(12):1299-304.

3. Nakayama T, ONo K, HAsHiMoTo K. Affinity of antioxidative polyphenols for lipid bilayers evaluated with a liposome system. Biosci, Biotechnol, Biochem. 1998; 62(5):1005-7.

4. Aggarwal B B, Harikumar K B. Potential therapeutic effects of curcumin, the anti-inflammatory agent, against neurodegenerative, cardiovascular, pulmonary, metabolic, autoimmune and neoplastic diseases. Int J Biochem Cell Biol. 2009; 41(1):40-59.

5. Ak T, Gilpin I. Antioxidant and radical scavenging properties of curcumin. Chem-Biol Interact. 2008; 174(1):27-37.

6. Swamy M V, Citineni B, Patlolla J M, Mohammed A, Zhang Y, Rao C V. Prevention and treatment of pancreatic cancer by curcumin in combination with omega-3 fatty acids. Nutr Cancer. 2008; 60(S1):81-9.

7. Dhillon N, Aggarwal B B, Newman R A, Wolff R A, Kunnumakkara AB, Abbruzzese J L, et al. Phase ii trial of curcumin in patients with advanced pancreatic cancer. Clin Cancer Res. 2008; 14(14):4491-9.

8. Condat M, Mazeran P-E, Malval J-P, Lalevée J, Morlet-Savary F, Renard E, et al. Photoinduced curcumin derivative-coatings with antibacterial properties. RSC Advances. 2015; 5(104):85214-24.

9. Mun S-H, Joung D-K, Kim Y-S, Kang O-H, Kim S-B, Seo Y-S, et al. Synergistic antibacterial effect of curcumin against methicillin-resistant staphylococcus aureus. Phytomedicine. 2013;20(8-9):714-8.

10. Tyagi P, Singh M, Kumari H, Kumari A, Mukhopadhyay K. Bactericidal activity of curcumin i is associated with damaging of bacterial membrane. PLoS ONE. 2015; 10(3):e0121313.

11. Mei L, McClements D J, Wu J, Decker E A. Iron-catalyzed lipid oxidation in emulsion as affected by surfactant, ph and nacl. Food Chem. 1998; 61(3):307-12.

12. Mei L, McClements D J, Decker E A. Lipid oxidation in emulsions as affected by charge status of antioxidants and emulsion droplets. J Agric Food Chem. 1999; 47(6):2267-73.

13. Warren D B, Chalmers D K, Hutchison K, Dang W, Pouton C W. Molecular dynamics simulations of spontaneous bile salt aggregation. Colloids Surf Physicochem Eng Aspects. 2006; 280(1-3):182-93.

14. Charoen R, Jangchud A, Jangchud K, Harnsilawat T, Naivikul O, McClements DJ. Influence of biopolymer emulsifier type on formation and stability of rice bran oil-in-water emulsions: Whey protein, gum arabic, and modified starch. J Food Sci. 2011; 76(1):E165-E72.

15. Dickinson E. Hydrocolloids as emulsifiers and emulsion stabilizers. Food Hydrocoll. 2009; 23(6):1473-82.

16. Pannell N. Microbial encapsulation. European Patent 1990; 242:135.

17. Czerniak A, Kubiak P, Bialas W, Jankowski T. Improvement of oxidative stability of menhaden fish oil by microencapsulation within biocapsules formed of yeast cells. J Food Eng. 2015; 167:2-11.

18. Paukner S, Kohl G, Lubitz W. Bacterial ghosts as novel advanced drug delivery systems: Antiproliferative activity of loaded doxorubicin in human caco-2 cells. J Controlled Release. 2004; 94(1):63-74.

19. Hamidi M, Zarrin A, Foroozesh M, Mohammadi-Samani S. Applications of carrier erythrocytes in delivery of biopharmaceuticals. J Controlled Release. 2007; 118(2):145-60.

20. Young S, Dea S, Nitin N. Vacuum facilitated infusion of bioactives into yeast microcarriers: Evaluation of a novel encapsulation approach. Food Res Int. 2017; 100:100-12.

21. Rohlfs E M, Louie D S, Zeisel S H. Lipid synthesis and secretion by primary cultures of rat mammary epithelial cells. J Cell Physiol. 1993; 157(3):469-80.

22. Fox P F, McSweeney P L H. Advanced dairy chemistry: Springer; 2014.

23. McManaman J L. Formation of milk lipids: A molecular perspective. Journal of Clinical Lipidology 2009; 4(3):391-401.

24. Michalski M C. Specific molecular and colloidal structures of milk fat affecting lipolysis, absorption and postprandial lipemia. Eur J Lipid Sci Technol. 2009; 111(5):413-31.

25. MacGibbon A, Taylor M. Composition and structure of bovine milk lipids. Advanced dairy chemistry volume 2 lipids: Springer; 2006. p. 1-42.

26. Berton-Carabin C C, Ropers MH, Genot C. Lipid oxidation in oil-in-water emulsions: Involvement of the interfacial layer. Comprehensive Reviews in Food Science and Food Safety. 2014; 13(5):945-77.

27. Liu H-X, Adachi I, Horikoshi I, Ueno M. Mechanism of promotion of lymphatic drug absorption by milk fat globule membrane. Int J Pharm. 1995; 118(1):55-64.

28. Kanno C. Emulsifying properties of bovine milk fat globule membrane in milk fat emulsion: Conditions for the reconstitution of milk fat gobules. J Food Sci. 1989; 54(6):1534-9.

29. Farhang B. Encapsulation of bioactive compounds in liposomes prepared with milk fat globule membrane-derived phospholipids. Guelph, Ontario, Canada: The University of Guelph; 2013.

30. Gallier S, Gragson D, Jimenez-Flores R, Everett D. Using confocal laser scanning microscopy to probe the milk fat globule membrane and associated proteins. J Agric Food Chem. 2010; 58(7):4250-7.

31. Tikekar R V, Pan Y, Nitin N. Fate of curcumin encapsulated in silica nanoparticle stabilized pickering emulsion during storage and simulated digestion. Food Res Int. 2013; 51(1):370-7.

32. Sarkar A, Goh K K T, Singh R P, Singh H. Behaviour of an oil-in-water emulsion stabilized by β-lactoglobulin in an in vitro gastric model. Food Hydrocoll. 2009; 23(6):1563-9.

33. Minekus M, Alminger M, Alvito P, Ballance S, Bohn T, Bourlieu C, et al. A standardised static in vitro digestion method suitable for food - an international consensus. Food & Function. 2014; 5(6):1113-24.

34. Sarkar A, Horne D S, Singh H. Interactions of milk protein-stabilized oil-in-water emulsions with bile salts in a simulated upper intestinal model. Food Hydrocoll. 2010; 24(2-3):142-51.

35. Porter C J, Trevaskis N L, Charman W N. Lipids and lipid-based formulations: Optimizing the oral delivery of lipophilic drugs. Nature Reviews Drug Discovery. 2007; 6(3):231-48.

36. Tønnesen HH, Masson M, Loftsson T. Studies of curcumin and curcuminoids. Xxvii. Cyclodextrin complexation: Solubility, chemical and photochemical stability. Int J Pharm. 2002; 244(1-2):127-35.

37. Ingram L O. Ethanol tolerance in bacteria. Crit Rev Biotechnol. 1989; 9(4):305-19.

38. Komatsu H, Okada S. Effects of ethanol on permeability of phosphatidylcholine/cholesterol mixed liposomal membranes. Chem Phys Lipids. 1997; 85(1):67-74.

39. Bourassa P, Kanakis C, Tarantilis P, Pollissiou M, Tajmir-Riahi H. Resveratrol, genistein, and curcumin bind bovine serum albumin J Phys Chem B. 2010; 114(9):3348-54.

40. Jaruga E, Sokal A, Chrul S, Bartosz G. Apoptosis-independent alterations in membrane dynamics induced by curcumin. Exp Cell Res. 1998; 245 (2):303-12.

41. Khajavi M, Shiga K, Wiszniewski W, He F, Shaw C A, Yan J, et al. Oral curcumin mitigates the clinical and neuropathologic phenotype of the trembler-j mouse: A potential therapy for inherited neuropathy. The American Journal of Human Genetics. 2007; 81(3):438-53.

42. Sun Y, Lee C-C, Hung W-C, Chen F-Y, Lee M-T, Huang H W. The bound states of amphipathic drugs in lipid bilayers: Study of curcumin Biophys J. 2008; 95(5) :2318-24.

43. Barry J, Fritz M, Brender J R, Smith P E, Lee D-K, Ramamoorthy A. Determining the effects of lipophilic drugs on membrane structure by solid-state nmr spectroscopy: The case of the antioxidant curcumin J Am Chem Soc. 2009; 131(12) :4490-8.

44. Pan Y, Tikekar R V, Nitin N. Effect of antioxidant properties of lecithin emulsifier on oxidative stability of encapsulated bioactive compounds. Int J Pharm. 2013; 450(1-2):129-37.

45. Kharat M, Du Z, Zhang G, McClements D J. Physical and chemical stability of curcumin in aqueous solutions and emulsions: Impact of ph, temperature, and molecular environment. J Agric Food Chem. 2017; 65(8):1525-32.

46. McClements D J. Crystals and crystallization in oil-in-water emulsions: Implications for emulsion-based delivery systems. Adv Colloid Interface Sci. 2012; 174:1-30.

47. Armand M, Borel P, Pasquier B, Dubois C, Senft M, Andre M, et al. Physicochemical characteristics of emulsions during fat digestion in human stomach and duodenum. Am J Physiol Gastrointest Liver Physiol. 1996; 271(1):G172-G83.

48. Mun S, Decker E A, Park Y, Weiss J, McClements D J. Influence of interfacial composition on in vitro digestibility of emulsified lipids: Potential mechanism for chitosan's ability to inhibit fat digestion. Food Biophys. 2006; 1(1):21-9.

49. Gallier S, Ye A, Singh H. Structural changes of bovine milk fat globules during in vitro digestion. J Dairy Sci. 2012; 95(7):3579-92.

50. Roman C, Carriere F, Villeneuve P, Pina M, Millet V, Simeoni U, et al. Quantitative and qualitative study of gastric lipolysis in premature infants: Do mct-enriched infant formulas improve fat digestion? Pediatr Res. 2007; 61(1):83.

51. Bernback S, Blackberg L, Hernell O. Fatty acids generated by gastric lipase promote human milk triacylglycerol digestion by pancreatic colipase-dependent lipase. Biochimica et Biophysica Acta, Lipids and Lipid Metabolism. 1989; 1001(3):286-93.

52. Armand M. Milk fat digestibility. Sciences des Aliments. 2008; 28(1-2): 84-98.

53. Gargouri Y, Pieroni G, Lowe PA, Sarda L, Verger R. Human gastric lipase: The effect of amphiphiles. Eur J Biochem. 1986; 156(2):305-10.

54. Gargouri Y, Moreau H, Verger R. Gastric lipases: Biochemical and physiological studies. Biochimica et Biophysica Acta, Lipids and Lipid Metabolism. 1989; 1006(3):255-71.

55. Hamosh M, Scanlon J W, Ganot D, Likel M, Scanlon K B, Hamosh P. Fat digestion in the newborn: Characterization of lipase in gastric aspirates of premature and term infants. J Clin Invest. 1981; 67(3):838-46.

56. Dressman J B, Berardi RR, Dermentzoglou L C, Russell T L, Schmaltz S P, Barnett J L, et al. Upper gastrointestinal (gi) ph in young, healthy men and women. Pharm Res. 1990; 7(7):756-61.

57. Piper D, Fenton B H. Ph stability and activity curves of pepsin with special reference to their clinical importance. Gut. 1965; 6(5):506.

58. Suwannateep N, Banlunara W, Wanichwecharungruang S P, Chiablaem K, Lirdprapamongkol K, Svasti J. Mucoadhesive curcumin nanospheres: Biological activity, adhesion to stomach mucosa and release of curcumin into the circulation. J Controlled Release. 2011; 151(2):176-82.

59. Young S. The physicochemical properties of yeast microcarriers: University of California, Davis; 2018.

60. Paramera E I, Konteles S J, Karathanos V T. Stability and release properties of curcumin encapsulated in saccharomyces cerevisiae, β-cyclodextrin and modified starch. Food Chem. 2011; 125(3):913-22.

61. Zhou M, Hu Q, Wang T, Xue J, Luo Y. Characterization of high density lipoprotein from egg yolk and its ability to form nanocomplexes with chitosan as natural delivery vehicles. Food Hydrocoll. 2018; 77:204-11.

62. Gallier S, Cui J, Olson T D, Rutherfurd S M, Ye A, Moughan P J, et al. In vivo digestion of bovine milk fat globules: Effect of processing and interfacial structural changes. I. Gastric digestion. Food Chem. 2013; 141(3):3273-81.

63. Vanderghem C, Francis F, Danthine S, Deroanne C, Paquot M, Pauw E D, et al. Study on the susceptibility of the bovine milk fat globule membrane proteins to enzymatic hydrolysis and organization of some of the proteins. Int Dairy J. 2011; 21(5):312-8.

64. Ye A, Cui J, Singh H. Proteolysis of milk fat globule membrane proteins during in vitro gastric digestion of milk. J Dairy Sci. 2011;94(6):2762-70.

65. Pool H, Mendoza S, Xiao H, McClements D J. Encapsulation and release of hydrophobic bioactive components in nanoemulsion-based delivery systems: Impact of physical form on quercetin bioaccessibility. Food & Function. 2013; 4(1):162-74.

66. Yao M, Xiao H, McClements DJ. Delivery of lipophilic bioactives: Assembly, disassembly, and reassembly of lipid nanoparticles. Annu Rev Food Sci Technol. 2014;5:53-81.

67. McClements D J, Saliva-Trujillo L, Zhang R, Zhang Z, Zou L, Yao M, et al. Boosting the bioavailability of hydrophobic nutrients, vitamins, and nutraceuticals in natural products using excipient emulsions. Food Res Int. 2015.

68. Patton JS, Carey MC. Watching fat digestion. Science. 1979; 204(4389):145-8.

69. Gallier S, Zhu XQ, Rutherfurd S M, Ye A, Moughan P J, Singh H. In vivo digestion of bovine milk fat globules: Effect of processing and interfacial structural changes. Ii. Upper digestive tract digestion. Food Chem. 2013; 141(3):3215-23.

70. Bong P H. Spectral and photophysical behaviors of curcumin and curcuminoids. Bull Korean Chem Soc. 2000; 21(1):81-6.

71. Priyadarsini K I. Photophysics, photochemistry and photobiology of curcumin: Studies from organic solutions, bio-mimetics and living cells. J Photochem Photobiol C: Photochem Rev. 2009; 10(2):81-95.

### Gastrointestinal Digestion of Curcumin Encapsulated in Oleosome

### Background

Plant polyphenols have been of special interest in recent years due to their ubiquitous metabolites that possess various biological activities, such as antioxidant and antitumor properties. Curcumin, a yellow polyphenolic phytochemical from rhizomes of turmeric spice, is a Ayurvedic medicine for centuries. It has shown protection against various pro-inflammatory diseases including cancer, diabetes, cardiovascular disease, arthritis and gastric inflammation (Gupta, 2013). Also, it is not toxic to humans at very high doses (Lao, 2006). However, clinical trials show that curcumin exhibit poor bioavailability due to poor absorption, degradation in alkaline pH conditions, rapid metabolism and elimination (Anand, 2007). Besides, curcumin has low water solubility (11 ng/ml), making it difficult to be incorporated into food system (Tonnesen, 2002).

Numerous carriers have been made in increasing curcumin solubility, ameliorate its release and enhance oral bioaccessibility. In food applications, carriers can be protein, carbohydrate or lipid based. Among them, lipid carrier is the least toxic in vivo applications (Puri, 2010). Examples of lipid carriers include emulsion, liposome, solid lipid nanoparticle and nanostructure lipid carrier. Lipid carriers have been extensively studied in curcumin encapsulation. For example, liposome prepared from microfluidization of lecithin improved bioavailability of curcumin in rats (Takahashi, 2009). Among these lipid carriers, emulsion is one of the most promising techniques for delivery of curcumin. When encapsulated in medium chain triglyceride droplet with the stabilization of whey protein and Tween-80, curcumin showed resistance to pepsin digestion and slow release in pancreatic digestion (Sari, 2015). Co-delivery of curcumin and catechin into water in oil in water double emulsion increased its bioaccessibility (Aditya, 2015). Silica nanoparticle stabilized Pickering emulsion enhanced curcumin release in a simulated intestinal digestion (Tikekar, 2013). Although bioaccessibility has been improved in these studies, common encapsulation technologies require complicated preparation techniques, such as microfluidization and homogenization as well as toxic or not-natural materials (Fathi, 2012).

An alternative preformed carrier has been proposed in bioactive compound encapsulation. In fact, nature has created many cellular compartments that are potential delivery carriers. Several tries to utilize existing capsules are limited to spores, plant cells, pollen grains and yeast (Pham-Hoang, 2015). Despite the high protection provided by capsules, these biological derived systems have shown several disadvantages. For example, extracellular compounds are very difficult to load into spores and plant cells. Pollen grains usually needs to be emptied before loading (Pham-Hoang, 2015). In yeast cell, plasma membrane confines the selective permeability to the molecules produced by the living cell. Physical or chemical methods are often needed to perturb the plasma membrane and internalize the bioactive compound (Pedrini, 2014).

Another natural carrier that may have promising applications in food delivery is oleosome. Oleosome is plant storage organelles existed in various oil bearing seeds, such as soybean and almond. Structurally, oleosome resembles conventional protein stabilized oil in water emulsion. It consists of an inner oil core and an outer layer of phospholipid which is surrounded by an interfacial protein layer. The protein layer is consisted of three types of protein including oleosin, caleosin and steroleosin with oleosin as the most abundant protein (Chapman, 2012). Oleosin contains three domains, with one domain covering the phospholipid head group, one domain penetrating the phospholipid monolayer and the third domain that is anchored in the triglyceride core.

In this study, oleosome was used as a preformed carrier to encapsulate curcumin. Meanwhile, encapsulation of curcumin into whey protein stabilized emulsion was also investigated as comparison. Milk components including casein, whey protein, lactose and anhydrous milk fats are widely used for making emulsion in food industry (Vega, 2006). Whey protein is isolated from whey, the by-product of cheese production. It is amongst one of the most employed emulsifiers in food. The physical properties and functional properties of whey protein have been well characterized (Katsuta,1990; Leman, 1989). Whey protein has also been extensively studied as wall material for bioactive compounds in both emulsion forms and gel forms (Betz, 2011; Sari, 2015). Curcumin nanoemulsion encapsulated in whey protein concentration-70 and Tween 80 showed relative resistance to pepsin digestion, but not pancreatin, indicating increased bioavailability (Sari, 2015). Bioaccessibility of curcumin in oleosome during gastrointestinal digestion was compared to whey protein stabilized emulsion. Besides, stability of the two encapsulation systems against oxidative stress was studied.

In some embodiments, a natural delivery system, oleosome extracted from almond milk, was used to encapsulate curcumin by simple incubation. Encapsulation efficiency achieved 70% after 5min incubation. Curcumin encapsulated in oleosome showed around 56% release during in vitro gastric digestion, possibly due to the cleavage of oleosin by pepsin. Direct intestinal digestion as well as sequential intestinal digestion showed rapid and around 90% release of curcumin The gastrointestinal release of curcumin was shown to be close to whey protein emulsion. Curcumin encapsulated in oleosome showed significantly higher oxidative stability as compared to WPI counterpart. This study provides preliminary evidences showing potential applications in using oleosome as encapsulation capsules in food and pharmaceutical applications.

### Curcumin Encapsulation

Curcumin has been shown to bind directly to numerous signaling molecules, DNA, RNA and carrier proteins. Curcumin has demonstrated affinity with several carrier proteins including casein, human serum albumin, bovine serum albumin, immunoglobulin and β-Lactoglobulin through hydrophobic interaction, which enhanced its bioavailability (Gupta, 2011). For example, β-Lactoglobulin is capable of binding curcumin within its central cavity (Sneharani, 2010). Oleosin protein is consisted of hydrophilic strands that are adsorbed on the phospholipid interface as well as a long hydrophobic strand inserting into triglyceride core. Curcumin was internalized into the oleosome core, possibly due to the hydrophobic interaction as well as diffusion.

### Gastric Digestion

Curcumin has been encapsulated in artificial oleosome and showed higher bioaccessibility compared to non-encapsulated control in rat model (Chang, 2013). Oleosome structure is stabilized by oleosin protein. The intact structure of oleosin is critical for oleosome stability. SDS-PAGE of oleosome after incubated with pepsin showed a loss of oleosin bands indicating pepsin can degrade oleosin (White, 2009; Makkhun, 2015). Particularly, hydrophilic residues on oleosin located on surface of the interface were shown to be susceptible to pepsin degradation. Stability loss in the gastric fluid led to the flocculation and or coalescence of oleosome (Gallier, 2012). This is consistent with particle size measurement, which showed the appearance of peak at around 30um after gastric digestion. On the other hand, flocculation may block the accessibility of oleosin and prevent further degradation, which may explain the partial curcumin release in gastric digestion.

Like oleosome, whey protein is partially susceptible to gastric digestion. Whey protein is consisted of major two whey proteins beta-lactoglobulin and alpha-lactoglobulin. Alpha-lactoglobulin is susceptible to pepsin treatment. Beta-lactoglobulin is resistant to pepsin digestion in solution, but it is more susceptible when absorbed onto interface due to conformation rearrangement and unfolding (Sarkar, 2009b). Unlike oleosome, WPI was reported to stay stable against aggregation and coalescence under gastric condition (Li, 2012; Thesis, in vitro gastrointestinal digestion of oil in water emulsions and Malaki Nik, 2002). It was suggested that excess proteins in serum phase as well as protein hydrolyzed peptides products may have binded to the interface and contributed to the stability. This is consistent with particle size measurement where no larger particle was observed after gastric treatment.

### Intestinal Digestion

Curcumin is prone to rapid degradation in buffer systems. Release of curcumin is not favored during gastric digestion. In intestinal digestion, incorporation of curcumin into the middle micellar phase is preferred. The micellar phase is considered as a major digestible portion. Burst release of curcumin was seen in both oleosome and WPI in intestinal digestion. WPI nanoemulsion encapsulated with curcumin was reported to have 5% release under gastric digestion and 70% release under intestinal digestion (Sari, 2015). In intestinal digestion, bile salt imparted charge for the digestion mixture and rendered them with higher stability, which was shown in the decrease of zeta-potential after intestinal digestion. The binding of bile salt onto interface facilitated the binding of lipase.

Makkhun and coworkers showed that oleosin can be displaced by bile salt (Makkhun, 2015). Bile salt displacement rendered oleosome structure more susceptible to lipase digestion. Beta-lactoglobulin absorbed at the interface can be almost completely displaced by bile salt via an orogenic mechanism (Maldonado-Valderrama, 2008). Higher curcumin release after direct intestinal digestion in oleosome than WPI suggested that beta-lactoglobulin is more resistant to bile salt displacement.

The high release of curcumin during the intestinal digestion may also be associated with long chain fatty acid in oleosome oil. It was reported that curcumin has very low bioavailability when encapsulated in short chain fatty acid. Using emulsion containing middle or long chain triacylglycerol substantially increased its bioavailability because of the formation of mixed micelles, which can solubilize highly lipophilic compound. (Ahmed, 2012). Curcumin encapsulated in whey protein emulsion prepared from long chain triacylglycerol/corn oil showed 50% bioaccessibility. Chen and coworkers also showed bioaccessibility was relatively around 66% in nanoemulsions made from long chain triglycerides (Chen, 2012). In our study, the overall bioaccessibility could be calculated from curcumin retained after gastric digestion multiplied by intestinal digestion release percentage, which would be close to 50%. Almond oil is mainly composed of palmitic acid, palmitoleic acid, stearic acid, oleic acid, linoleic acid (Leo, 2015). These long chain triacyclglycerols may have contributed to high release of curcumin in intestinal digestion. Also, the similar release profiles between whey protein emulsion and oleosome may indicate important role of core structure rather than interface in determining the release.

### AAPH

Oleosome showed high oxidative stability after freezing, freeze-drying or heating treatment (Kapchie, 2013; Chen, 2012). It was suggested that oxidative stability in oleosome is closely associated with oleosin protein, which is tightly covers the whole oleosome surface together with phospholipid membrane. It was reported that oleosome membrane structure prevented oil from coalescing during seed desiccation (Froese, 2003). Pan and coworkers demonstrated that antioxidative property of emulsifiers significantly contributed to the free radical permeation across interface (Pan, 2013). In addition to oleosin protein layer, oleosome structure contains a layer of phospholipid. The major phospholipid in oleosome is antioxidative phosphatidylcholine (Garcia, 1988), which may have contributed to higher stability of curcumin encapsulated in oleosome over WPI. The local distribution of bioactive compound also impacts oxidative stability. Exclusion of beta-carotene in solid lipid nanoparticles corresponded to a lower stability compared to emulsion where beta-carotene distributed evenly inside (Pan, 2016). In current study, both in oleosome and WPI, curcumin had a uniform distribution across inside the core. Curcumin distribution may be the contributing factor. Artificial oil body also showed higher resistance to lipid oxidation than emulsion prepared with tuna oil with Tween40, sodium caseinate, and commercial canola protein isolate as emulsifiers (Wijesundera, 2013).

### EXAMPLES

The following examples are non-limiting and other variants within the scope of the art also contemplated.

### Example 44—Oleosome Isolation

Oleosome was isolated with a modified method proposed by Chen and Ono (Chen, 2010). Almonds bought from supermarket were soaked in deionized water for more than 20 h. After this, almonds were separated and fresh deionized water was added. The mixture was ground with Osterizer 10 speed blender. Almond milk was obtained by filtering the slurry with three layers of Kimtech science tissue wipers. Sucrose was added into almond milk at a 1:3 sucrose-to-almond milk ratio and pH of the mixture was adjusted to 11. Mixture was then centrifuged at 15,000 g for 30min at 4-degree C. Oleosome cream layer was obtained with a spoon. Cream layer was then resuspended in 20% w/w sucrose solution, pH 11 and centrifuged at 15,000 g for 30min at 4-degree C. This washing step was repeated for one more time.

### Example 45—Almond Oil Extraction

Almond oil was extracted from isolated oleosome with a method proposed by Leo (Leo, 2005). 20-30 ml hexane/methanol (2:1) was added to lg of oleosome. The mixture was vortexed and then centrifuged at 3000 g for 10 min. The upper layer was separated off and then evaporated under vacuum overnight to obtain almond oil.

### Example 46—Fluorescence Staining

To study the intactness of oleosome in 5% ethanol, oleosome dissolved in 5% ethanol was stained with lmg/ml Nile red (Sigma Aldrich) in DMSO (1:1000 v/v) and 4 mg/ml FITC (Life technologies, Eugene, Oreg.) in DMSO (1:10000 v/v). Images were taken with fluorescent microscope (Olympus IX71) with 100× oil objective.

### Example 47—Confocal Laser Scanning Microscopy

The microstructure of encapsulated oleosome and whey protein emulsion was studied with Confocal laser scanning microscopy (Leica, Germany) with 100× oil immersion objective lens. Nile red (X, X) was used (concentration and solvent) to stain triglyceride.

### Example 48—Encapsulation of Curcumin into Oleosome

2.5 mg/ml curcumin was dissolved in 250 ul pure ethanol and then added to 4.75 ml Phosphate buffer containing lg oleosome. Control group was prepared in the same method in the absence of curcumin Mixture was vortexed and then either directly separated or incubated at RT for 30 min before separation. Separation was conducted by centrifugation at 10,000 g for 10 min. Oleosome cream was obtained and then resuspended in 5% ethanol and followed by centrifugation at 10,000 g for 10 min.

### Example 49—Oleosome Encapsulation Efficiency

Oil content in 50 mg oleosome was calculated both before and after encapsulation. The amount of curcumin encapsulated in oleosome was determined by methanol extraction. Before and after encapsulation, 25 mg oleosome was mixed with lml methanol. Samples were vortexed, bath sonicated for 10min and then centrifuged at 161000 g for 10 min 500 ul of the supernatants were pipetted into cuvette and absorbance was read with wavelength of 425 nm with UV-Vis spectrophotometer (GENESYS 10S Series, Thermo Scientific). Control group was used as a blank.

The encapsulation efficiency (EE) was calculated as below:

EE=Me/Moil1*Mol2*40/625

Me is the mass of curcumin in 25 mg encapsulated oleosome in the unit of ug. Moill is the mass of oil in 25 mg encapsulated oleosome in the unit of mg. Moil2 is the mass of oil in 25 mg oleosome in the unit of mg before encapsulation.

### Example 50—Whey Protein Emulsion Preparation and Encapsulation

A 2% w/v stock solution of whey protein solution was prepared and then pH was adjusted to 6.5. Curcumin was added to almond oil at lmg/g. The mixture was vortexed and heat at 80-degree C. for 10min and then centrifuged at 16100 g for 10min to remove undissolved curcumin. The mixture was then added to 20 ml whey protein stock solution. Emulsion was prepared by dispersing the mixture at 9600 rpm for 2min, followed by sonication at 50% Amplitude for 30 secs.

### Example 51—Simulated Gastric Digestion of Whey Protein

Simulated gastric digestion was conducted based on a modified method reported by Sarkar (Sarkar, 2009). 1L 0.5% NaCl solution and 5 ml 5% NaCl solution were prepared and pH was adjusted to 2. Both solutions were warmed at 37-degree C. After this, 32 mg/ml pepsin was added to 5% NaCl.

For whey protein, pH of encapsulated whey protein was adjusted to 2. Then, 9 ml encapsulated whey protein (pH 2) was mixed with 1 ml 5% NaCl containing 32 mg/ml pepsin (pH 2). Sample was vortexed. 0.2 ml of the mixture was pipetted out immediately to measure the initial curcumin absorbance (TO). The digestion mixture was then placed in a dialysis bag. The bag was placed in 1L 0.5% NaCl solution (pH 2) and maintained at 37-degree C with a slow stirring speed. Duplicates of 0.2 ml emulsion was sampled every 0.5h. Samples from all time points were extracted with 0.5 ml methanol and vortexed. After sonication for 10min, samples were centrifuged at 16100 g for 10 min and absorbance was read under 425 nm. Non-encapsulated whey protein was conducted in the same method and was used as a blank.

### Example 52—Simulated Gastric Digestion of Oleosome

1.1 L 0.5% NaCl solution was prepared and pH was adjusted to 2. Solution was warmed at 37-degree C. 200 mg oleosome was mixed with 10 ml 0.5% NaCl solution containing pepsin containing 3.2 mg/ml pepsin (pH 2). The digestion was conducted as described above.

### Example 53—Intestinal Digestion of Whey Protein

20 ml 2× simulated intestinal digestion fluid (SIF) was prepared by dissolving 6.66 mg/ml CaCl₂, 40 mg/ml bile salt in 50mM KH₂PO₄. Solution was centrifuged at 3000 g for 5min to remove aggregation. pH of the solution was adjusted to 7.5 with NaOH. 4 mg/ml Lipase was added into the solution and incubated at 37-degree C for at least 15mins

pH of Whey protein solution was adjusted to 7.5. 7 ml encapsulated whey protein solution was mixed with 7 ml 2×SIF. Duplicates of 0.2 ml mixture were pipetted out immediately and extracted with 0.5 ml methanol to determine the total amount of curcumin Digestion mixture was incubated at 37-degree C with shaking speed at 145 rpm and 0.5 ml was sampled every 0.5 h. Samples were centrifuged at 16100 g for 10 min to obtain 0.2 ml of the micellar phase. The micellar phase was then extracted with methanol and absorbance was read under 425 nm.

### Example 54—Intestinal Digestion of Oleosome

2× simulated intestinal digestion fluid (SIF) was prepared as above. 10 ml deionized water was adjusted to pH 7.5 with NaOH. 200 mg oleosome was added. 7 ml oleosome in water (pH 7.5) was mixed with 7 ml SIF and digestion was conducted in the same way as above. The micellar phase was sometimes covered by upper cream layer. To prevent contamination, middle phase was taken with a syringe.

### Example 55—Sequential Digestion

After gastric digestion, gastric digestion mixture was adjusted to pH 7.5 with 1M NaHCO₃. Then, 7 ml gastric digested solution was mixed with 7 ml 2×SIF and intestinal digestion was conducted as described above.

### Example 56—Particle Size Measurement

Particle size distribution was measured with Microtrac (Microtrac, Montgomeryville, Pa.). For digested oleosome and whey protein emulsion, pH was adjusted to 7.5 before measurement. Encapsulated group was used for measuring the particle size.

### Example 57—Zeta Potential

Zeta potential was measured with Malvern Zetasizer Nano ZS instrument (Malvern Instruments Ltd, Worcestershire, UK). For digested oleosome and whey protein emulsion, pH was adjusted to 7.5 before measurement. Encapsulated group was used for measuring the particle size. Samples were placed in electrophoresis cell (Model DTS, Malvern Instruments Ltd). X readings from an individual sample were collected.

### Example 58—AAPH Measurements

100 mg/ml encapsulated oleosome and encapsulated whey protein emulsion were respectively added with 40mM AAPH (Sigma) or water as control. Encapsulated oleosome and whey protein emulsion were then rotated and curcumin was extracted at different time points.

### Example 59—Curcumin Encapsulation

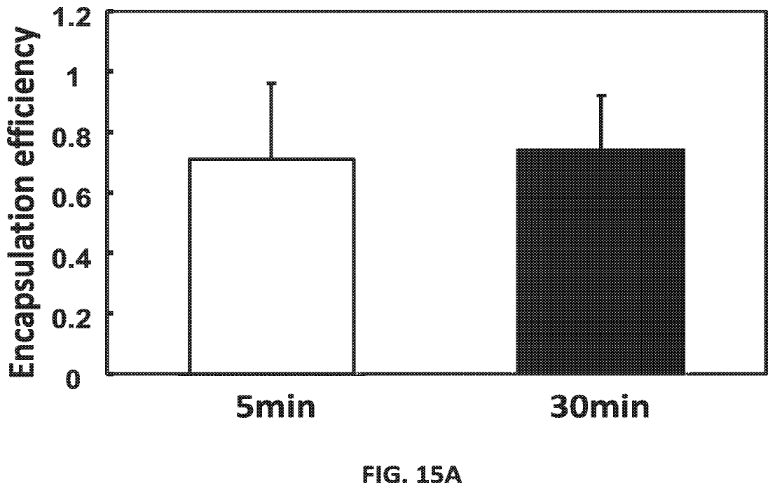

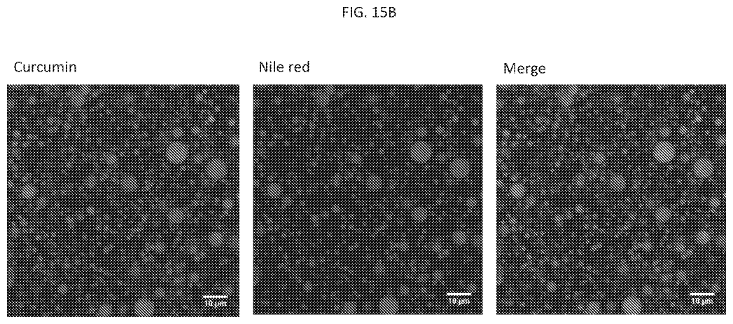

Curcumin was encapsulated in oleosome by incubation. FIG. 15A curcumin encapsulation efficiency in oleosome with different incubation time. There was no difference in encapsulation efficiency when longer incubation was applied. Around 70% encapsulation efficiency was obtained for both incubation time. Confocal image (FIG. 15B) showed that oleosome remained intact after encapsulation. Curcumin was localized in the entire lipid sphere, indicating that curcumin internalized into the lipid core.

### Example 60—Curcumin Release in Gastric Digestion

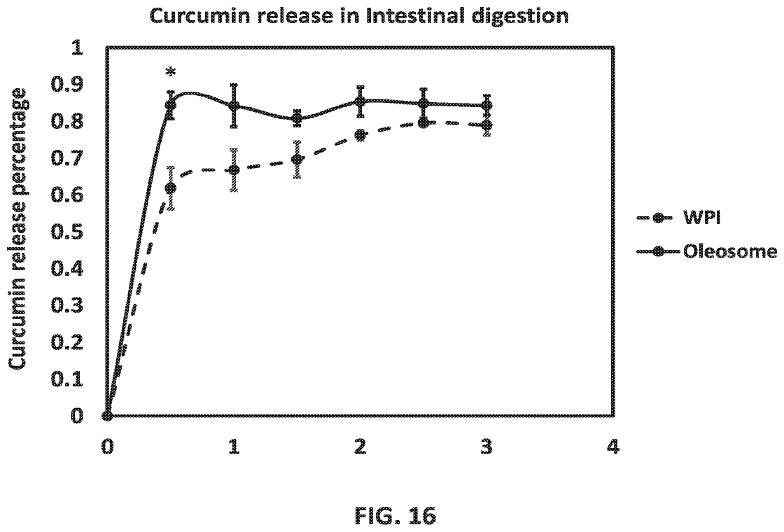

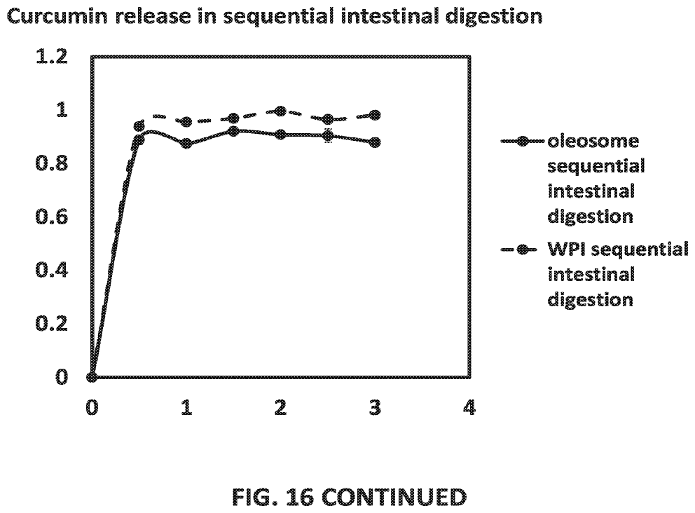

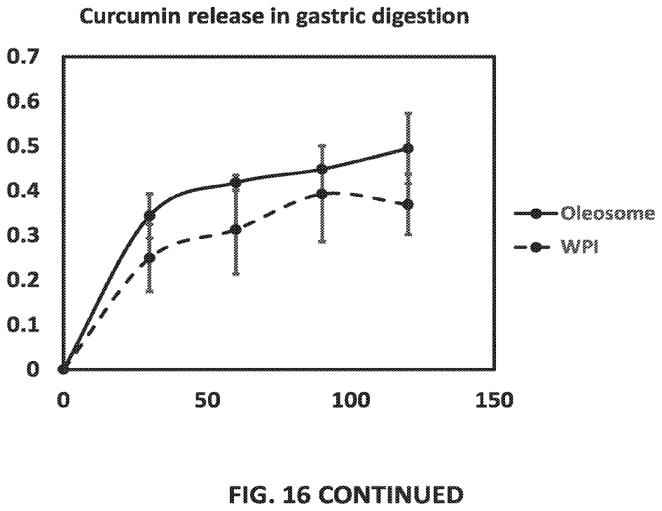

FIG. 16 showed curcumin release in simulated gastric digestion. In simulated gastric environment, curcumin release in oleosome and WPI were respectively 38% and 25% in the first 0.5 h and release increased with time. At 2 h time point, 56% curcumin was released in oleosome while 37% curcumin was released in WPI.

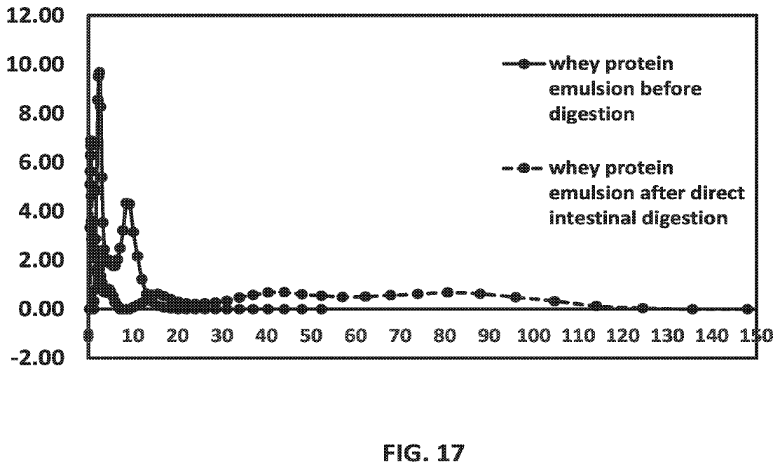

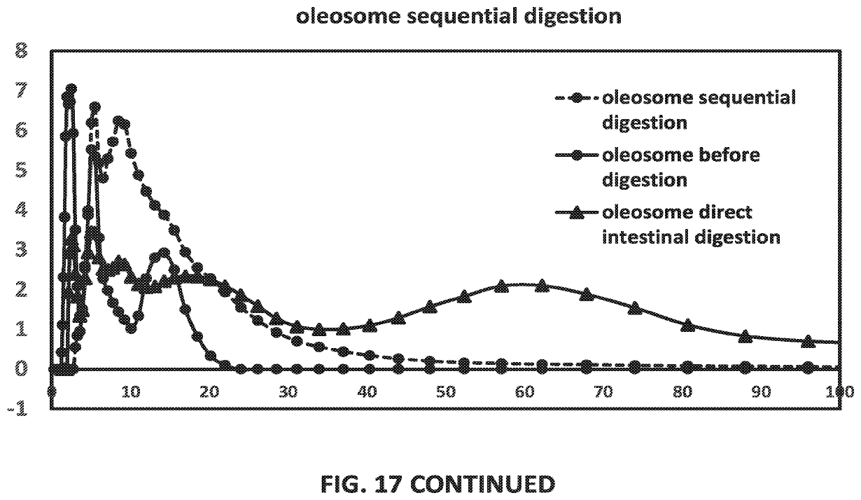

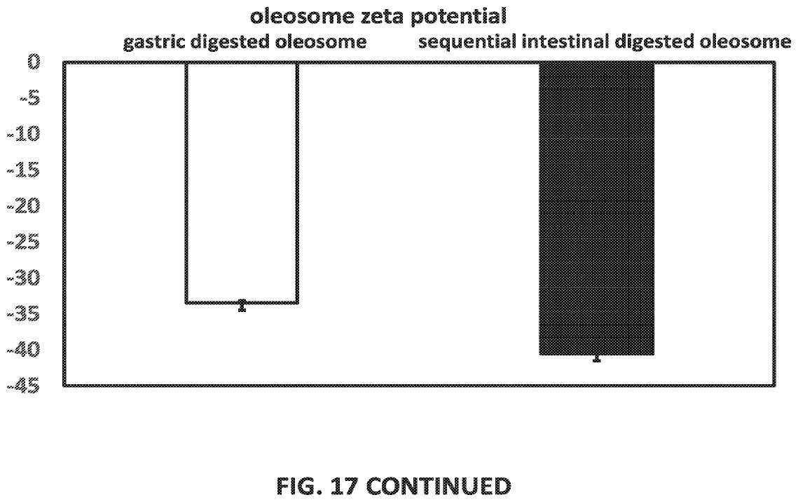

Before digestion, oleosome particle size distribution has three peaks: respectively at 2.5 um, 5 um and 13 um. Similar size distribution of oleosome was reported in Makkhun, 2015. Under gastric digestion, there was an increase in oleosome size, as shown in peak at 17 um. The size of WPI emulsion was relatively smaller than oleosome. Before digestion, WPI particle size has two peaks at 2.5 um and 9.25 um and it decreased after gastric digestion as shown by the diminish of 9.25 um peak. Zeta-potential of oleosome and WPI before and after digestion was shown in FIG. 17. Before digestion, zeta potential of oleosome and WPI were respectively −38.4 mV and −44.5mV, which suggested that both systems had good stability. After gastric digestion, both groups showed an increase in zeta potential, indicating lower stability of the two systems under gastric fluid.

### Example 61—Direct Intestinal Digestion

Curcumin release was measured after in vitro intestinal digestion. The digested micellar phase is separated from the indigestible oil phase as well as sediment phase by centrifugation. Direct intestinal digestion corresponded to a rapid release of curcumin in both oleosome and WPI emulsion with a slightly higher release percentage in oleosome than WPI. In 0.5 h, curcumin in oleosome reached 84% while it was 62% in WPI. In oleosome group, curcumin release remained constant over time while in WPI group, the release increased slightly over time.

Direct intestinal fluid treatment corresponded to decrease of volume percentage of particles including 2.5 um, Sum as well as appearance of a peak more than 60 um in oleosome group. In whey protein, the particle size decreased in intestinal digestion. Two major peaks were 0.5 um and 1.9 um respectively. Zeta potential was significantly decreased after intestinal fluid treatment in both oleosome and WPI groups. Oleosome showed a lower zeta potential than WPI.

### Example 62—Sequential Digestion

Both oleosome and WPI were pre-incubated with gastric fluid and then subjected to intestinal fluid. Curcumin release percentage was the ratio of curcumin absorbance after intestinal treatment to before. FIG. 17 showed that release percentage was 94% at 0.5 h and it increase to 98% at 3 h in WPI, higher than direct intestinal digestion. The release was close to direct intestinal digestion, with a release percentage of 94% at 0.5 h and then decreased slightly to 88% at 3 h in oleosome group. Pretreatment of WPI in gastric fluid increased curcumin release during subsequent intestinal digestion.

After sequential digestion, oleosome showed major two peaks in terms of particle size, which were at 5.5 um and 9.25 um respectively. WPI group showed decreased volume percentage at 2.75 um, 9.25 um as well as appearance of large particle at 22 um. Zeta-potential was −40.5 mV for oleosome, which was between zeta-potential obtained after gastric and direct intestinal digestion. In WPI group, Zeta-potential was −73.1 mV after sequential digestion, which was significantly lower than direct intestinal digestion treatment.

### Example 63—AAPH Measurement

Curcumin stability against oxidative stress was measured with AAPH assay. AAPH was used as a peroxyl radical generator. It has long half-life (approximately 175 h) in the aqueous solution (Zimowska, 1997). In oleosome control group, absorbance maintained above 90% within 46 h. In presence of AAPH, absorbance decreased to 76%, 68% and 42% in 20 h, 24 h and 46% respectively. For WPI control group, absorbance increased in 4 h and then decreased to 90% at 20 and 24 h. The presence of AAPH corresponded a rapid degradation at 20 h. Absorbance decreased to 22%, 12% and 0.03% in 20 h, 24 h and 46% respectively.

### Summary

This study showed that curcumin can be encapsulated in natural oleosome structure with an encapsulation efficiency of 70% by simple incubation. Curcumin showed similar in vitro gastrointestinal release profile when encapsulated in oleosome compared to encapsulated in WPI. Interestingly, oleosome showed higher protection for curcumin than WPI in the presence of AAPH. To the best of our knowledge, this is the time study using oleosome to encapsulate curcumin. This study may indicate potential applications of oleosome in food and pharmaceutical industries.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

What is claimed is:

1. A method for encapsulating at least one compound, comprising providing the at least one compound;
mixing a plurality of milk fat globules or oleosomes with the at least one compound to obtain a mix;
incubating the mix to allow the at least one compound to diffuse into at least one substructure of a fraction of the plurality of the milk fat globules or oleosomes comprising the at least one compound.

2. The method of claim 1, wherein the at least one compound is lipophilic or amphiphilic.

3. The method of claim 1, wherein the at least one compound is one or more of a bio-active compound, a colorant, or a flavor.

4. The method of claim 1, wherein the at least one compound is provided in a pharmaceutically acceptable solvent or food-grade solvent.

5. The method of claim 1, wherein the pharmaceutically acceptable solvent or food-grade solvent is selected from the group consisting of an organic solvent, a polar solvent, and a volatile oil.

6. The method of claim 1, wherein the plurality of milk fat globules or oleosomes is provided in water.

7. The method of claim 1, wherein the fraction of the plurality of milk fat globules or oleosomes comprising the at least one compound ranges from about 30% to about 100%.

8. The method of claim 1, wherein the milk fat globules are isolated from milk selected from the group consisting of bovine milk, ovine milk, goat milk, camel milk, and yak milk.

9. The method of claim 1, wherein the oleosome is from one or more of seeds, or nuts.

10. The method of claim 1, the milk fat globules or oleosomes are isolated by one or more of density, size, or polarity based methods.

11. The method of claim 10, wherein the density based method comprises centrifugation.

12. The method of claim 10, wherein the size based comprises filtration.

13. The method of claim 10, wherein the polarity based method comprises membrane and electric field induced separation methods.

14. The method of claim 4, wherein a concentration of pharmaceutically acceptable solvent or food-grade solvent ranges from about 0.1% v/v to about 10% v/v.

15. The method of claim 14, wherein the pharmaceutically acceptable solvent or food-grade solvent is ethanol.

16. The method of claim 15, wherein a concentration of ethanol is ≤10%v/v.

17. The method of claim 1, wherein the at least one compound has a log P value ranging from about 0.5 to about 10.

18. The method of claim 1, wherein the at least one compound has a molecular mass ranging from about 50 g/mol to about 5000 g/mol.

19. The method of claim 1, wherein the at least one compound is a food compound selected from the group consisting of lipid soluble vitamins (Vitamin A, D and K and their derivatives), flavors (Eugenol, Limonene, Vanillin, Rosemary, Dairy Flavors, and the like), Colors (Curcumin, annatto extract, capsaicin, and the like), Polyphenolic antioxidants (Flavonoids, Anthocynanins, Pro anthocyanidins, Curcuminoids, carotenoids, retinoids).

20. The method of claim 1, wherein the at least one compound is a pharmaceutical compound selected from the group consisting of HIV drugs, Cardiovascular drugs, antibiotic and antifungals, and oral anti-cancer drugs.

21. The method of claim 1, wherein the encapsulation of the at least one compound is quantified by a method selected from the group consisting of fluorescence microscopy, High Performance Liquid Chromatography, Liquid chromatography—mass spectrometry, High Performance Liquid chromatography—mass spectrometry, Gas chromatography—mass spectrometry, ultraviolet—visible spectroscopy or ultraviolet—visible spectrophotometry, ultraviolet—visible-near infrared spectroscopy or ultraviolet—visible spectrophotometry, Raman spectroscopy, and Fourier-transform infrared spectroscopy.

22. The method of claim 1, wherein the at least one compound displays a ring like distribution, peripheral distribution, homogenous distribution, or a combination thereof within the substructure of fraction of the plurality of milk fat globules.

23. The method of claim 1, wherein the incubating is performed a temperature range of about 4° C. to about 40° C.

24. The method of claim 1, wherein the incubating is performed a temperature range of about −10° C. to about 90° C.

25. The method of claim 1, wherein the incubating is performed for about 30 sec to about 50 h.

26. The method of claim 1, wherein the incubating is performed for about 10 min to about 60 min.

27. The method of claim 1, wherein the at least one compound is encapsulated at a concentration range of about 5 μg/g of milk fat to about 100 μg/g of milk fat.

28. The method of claim 1, wherein the at least one compound is encapsulated at a range of about 10% to about 100% of the dosage requirement per gm of milk fat.

29. The method of claim 1, wherein the at least one compound is provided at a concentration range of about 50 μM to about 2500 μM.

30. The method of claim 1, wherein the at least one compound is provided at a dose of about 0.01 mg/kg/day to about 1000 mg/kg/day.

31. A composition comprising:
a plurality of milk fat globules or oleosomes;
at least one compound;
an amount of a pharmaceutically acceptable solvent or food-grade solvent;
wherein the at least one compound is encapsulated within a fraction of the plurality of milk fat globules or oleosomes.

32. The composition of claim 31, wherein the at least one compound is lipophilic or amphiphilic.

33. The composition of claim 31, wherein the at least one compound is one or more of a bio-active compound, a colorant, or a flavor.

34. The composition of claim 31, wherein the at least one compound is provided in a pharmaceutically acceptable solvent or food-grade solvent.

35. The composition of claim 34, wherein the pharmaceutically acceptable solvent or food-grade solvent is selected from the group consisting of an organic solvent, a polar solvent, and a volatile oil.

36. The composition of claim 31, wherein a concentration of pharmaceutically acceptable solvent or food-grade solvent ranges from about 0.1 -10% v/v.

37. The composition of claim 31, wherein the pharmaceutically acceptable solvent or food-grade solvent is ethanol.

38. The composition of claim 37, wherein a concentration of ethanol is ≤10% v/v.

39. The composition of claim 31, wherein the at least one compound has a log P value ranging from about 0.5 to about 10.

40. The composition of claim 31, wherein the at least one compound has a molecular mass ranging from about 50 g/mol to about 5000 g/mol.

41. The composition of claim 31, wherein the at least one compound is a food compound selected from the group consisting of lipid soluble vitamins (Vitamin A, D and K and their derivatives), flavors (Eugenol, Limonene, Vanilin, Rosemary, Dairy Flavors, and the like), Colors (Curcumin, annatto extract, capsaicin, and the like), Polyphenolic antioxidants (Flavonoids, Anthocynanins, Pro anthocyanidins, Curcuminoids, carotenoids, retinoids).

42. The composition of claim 31, wherein the compound is a pharmaceutical compound selected from the group consisting of HIV drugs, Cardiovascular drugs, antibiotic and antifungals, and oral anti-cancer drugs.

43. The composition of claim 31, wherein the at least one compound is encapsulated at a concentration range of about 5 μg/g of milk fat to about 100 μg/g of milk fat.

44. The composition of claim 31, wherein the at least one compound is provided at a concentration range of about 50 μM to about 2500 μM.

45. The composition of claim 31, wherein the at least one compound is encapsulated at a range of about 10% to about 100% of the dosage requirement per gm of milk fat.

46. The composition of claim 31, wherein a size of the fraction of the plurality of milk fat globules or oleosomes comprising the at least one compound ranges from about 5 um to about 50 um.

47. The composition of claim 31, wherein a size of the fraction of the plurality of milk fat globules or oleosomes comprising the at least one compound ranges from about 0.1 um to about 50 um.

48. The composition of claim 31, wherein a size of the fraction of the plurality of milk fat globules or oleosomes comprising the at least one compound ranges from about 0.1 um to about 10 um.

49. The composition of claim 31, wherein the composition is for one or more of oral administration, transdermal administration, and topical administration.

50. The composition of claim 31, wherein the milk fat globules or oleosomes are present at a concentration of about 0.1% w/v to about 50% w/v.

51. A composition comprising:
a plurality of milk fat globules or oleosomes;
at least one compound;
an amount of a pharmaceutically acceptable solvent or food-grade solvent;
wherein the at least one compound is encapsulated within a fraction of the plurality of milk fat globules or oleosomes, and
wherein the composition is configured to deliver the compound at a site of interest.

52. The composition of claim 51, wherein the composition is configured to deliver the compound at a site of interest by gradually releasing the compound over a period of time.

53. The composition of claim 52, wherein the period of time ranges from about 2 h to about 24 h.

54. The composition of claim 52, wherein the period of time ranges from about 1 min to about 6 h.

55. The composition of claim 51, wherein the composition is configured to deliver the compound at a site of interest by immediately releasing the compound.

56. The composition of claim 51, wherein the fraction of the plurality of milk fat globules or oleosomes comprising the at least one compound ranges from about 30% to about 100%.

57. The composition of claim 51, wherein the site of interest is stomach, small intestine, large intestine, liver, skin, oral cavity, and teeth.

58. The composition of claim 51, wherein the at least one compound encapsulated within the plurality of milk fat globules or oleosomes is stabilized against acid degradation.

59. The composition of claim 51, wherein the at least one compound encapsulated within the plurality of milk fat globules or oleosomes is stabilized against acid degradation at a pH of about 0.5 to about 5.

60. The composition of claim 51, wherein a degradation/loss of activity one compound encapsulated within the plurality of milk fat globules or oleosomes under acidic pH conditions is determined by measuring a degradation product of the at least one compound.

61. The composition of claim 51, wherein a degradation/loss of activity one compound encapsulated within the plurality of milk fat globules or oleosomes under acidic pH conditions is determined by measuring a degradation product of the at least one compound by LC-MS.

62. The composition of claim 51, wherein a size of the fraction of the plurality of milk fat globules or oleosomes comprising the at least one compound ranges from about 5 um to about 50 um.

63. The composition of claim 51, wherein a size of the fraction of the plurality of milk fat globules or oleosomes comprising the at least one compound ranges from about 0.1 um to about 50 um.

64. The composition of claim 51, wherein a size of the fraction of the plurality of milk fat globules or oleosomes comprising the at least one compound ranges from about 0.1 um to about 10 um.

65. The composition of claim 51, wherein the composition is for one or more of oral administration, transdermal administration, and topical administration.

66. The composition of claim 51, wherein about 10% to about 80% of the at least one compound is released from the fraction of the plurality of milk fat globules comprising the at least one compound.

67. The composition of claim 51, wherein the at least one compound is provided at a dose of about 0.01 mg/kg/day to about 1000 mg//kg/day.

68. A composition comprising:
at least one compound encapsulated within a fraction of the plurality of milk fat globules or oleosomes by a method, comprising
providing a plurality of milk fat globules or oleosomes;
providing at least one compound;
mixing the plurality of milk fat globules or oleosomes with the at least one compound to obtain a mix;
incubating the mix to allow the at least one compound to diffuse into at least one substructure of a fraction of the plurality of milk fat globules or oleosomes comprising the at least one compound,
thereby encapsulating the at least one compound;
wherein the composition is configured to deliver the compound at a site of interest.

69. The composition of claim 68, wherein the composition provides thermal stability, oxidative stability, improved delivery, light stability, and pH stability to the at least one compound.

70. The composition of claim 68, wherein the composition provides biocompatibility to the at least one compound.

71. The composition of claim 68, wherein the composition provides extended stability to the at least one compound.

72. The composition of claim 68, wherein the composition reduces a toxicity of the at least one compound.

73. The composition of claim 68, wherein the composition provides a controlled release of the at least one compound.

74. A composition comprising:
an intact milk fat globule or oleosome;
at least one compound;
wherein the at least one compound is encapsulated within the intact milk fat globule.

75. The composition of claim 74, wherein the compound comprises at least one of a protein, an antioxidant, and an enzyme.

76. The composition of claim 74, wherein the composition is stable during gastric pass.

77. A pharmaceutical formulation comprising:
a composition of any one of claims 31-76, and
a pharmaceutically acceptable carrier.

78. The method of claim 1, wherein the at least one compound is provided as a micellar composition that fuses with the milk fat globule or oleosome.
